(19) United States
(12) Patent Application Publication
(10) Pub. No.: US 2026/0259497 A1
(43) Pub. Date: Sep. 3, 2026

(54) PREPARATION OF POLYMER, PREPARATION OF CHEMICALLY AMPLIFIED RESIST COMPOSITION, RESIST PATTERN FORMING PROCESS, POLYMER, AND CHEMICALLY AMPLIFIED RESIST COMPOSITION

(71) Applicant: Shin-Etsu Chemical Co., Ltd., Tokyo (JP)

(72) Inventors: Ryo Sudo, Joetsu-shi (JP); Masayoshi Sagehashi, Joetsu-shi (JP); Shuhei Kobayashi, Joetsu-shi (JP); Yoshihiro Tsuji, Joetsu-shi (JP)

(73) Assignee: Shin-Etsu Chemical Co., Ltd., Tokyo (JP)

(21) Appl. No.: 19/547,971

(22) Filed: Feb. 24, 2026

(30) Foreign Application Priority Data
Mar. 3, 2025 (JP) 2025-033041

(51) Int. Cl. G03F 7/039; C07C 309/12; C07C 381/12; C08F 22/10; G03F 7/00; G03F 7/004; G03F 7/038

(52) U.S. Cl. CPC G03F 7/0397; C07C 309/12; C07C 381/12; C08F 22/1006; G03F 7/0045; G03F 7/0046; G03F 7/0382; G03F 7/0384; G03F 7/70025; G03F 7/70341

(57) ABSTRACT

A method for preparing a polymer comprises the steps of (1) polymerizing starting monomers in a solution through living radical polymerization using a RAFT agent, to form a polymer P-1 having an end structure originating from the RAFT agent, (2) adding a radical generator and a thiol compound to the solution containing polymer P-1, and heating the solution to convert the end structure to hydrogen to form a polymer P, and (3) mixing the solution containing polymer P with a poor solvent for purification. The polymer having a narrow dispersity is prepared, sulfur-containing end groups originating from the RAFT agent are efficiently removed, and residual monomers and low degree-of-polymerization fractions are removed by purification.

### CROSS-REFERENCE TO RELATED APPLICATION

This non-provisional application claims priority under 35 U.S.C. § 119(a) on Patent Application No. 2025-033041 filed in Japan on Mar. 3, 2025, the entire contents of which are hereby incorporated by reference.

### TECHNICAL FIELD

This invention relates to a method for preparing a polymer, a method for preparing a chemically amplified resist composition, a resist pattern forming process, a polymer, and a chemically amplified resist composition.

### BACKGROUND ART

Pattern formation to a smaller feature size is required to meet the recent demand for higher integration density and operating speed of LSIs. Acid-catalyzed chemically amplified resist compositions are most often used in forming resist patterns with a feature size of 0.2 μm or less. High-energy radiation such as UV, deep-UV or EB is used as the energy source for exposure of these resist compositions. As the advanced miniaturization technology, the manufacture of microelectronic devices is performed in a large scale by the ArF immersion lithography including the exposure step with a liquid, typically water held between the projection lens and the substrate. Attempts are made to apply multi-patterning of ArF lithography and lithography using EUV of wavelength 13.5 nm.

For the preparation of a base polymer in chemically amplified resist compositions adapted for the ArF and EUV lithography processes, random polymerization using radical initiators is suitable as described in Patent Document 1.

To comply with the recent advance of the ArF immersion lithography and EUV lithography, the requirements on resist materials with better properties including high resolution, low roughness, and etching selectivity between exposed and unexposed regions become stricter. For satisfying these properties, it is demanded to prepare a polymer such that its molecular weight dispersity may be as narrow as possible, that is, a homogeneous base polymer with minimal variation.

The method for preparing polymers with a narrow dispersity is generally divided into two approaches. One approach is a purifying method including the steps of preparing a polymer by random polymerization and removing a fraction having a low degree of polymerization and unreacted monomers from the polymer by re-precipitation. The other approach is a method including the step of conducting polymerization reaction while controlling the degree of polymerization uniform, for thereby obtaining a polymer with a narrow dispersity.

When a polymer is prepared by random polymerization, there is a likelihood that polymers having different degrees of polymerization form in the polymerization system. It is then difficult to obtain a polymer having a narrow dispersity. For obtaining a polymer with a narrow dispersity (suited as the base polymer for resist compositions) in the reaction step, living radical polymerization, especially using a reversible addition-fragmentation chain transfer agent (referred to as RAFT agent, hereinafter) is proposed in the art (Patent Documents 2, 3, 4 and 5).

In the living radical polymerization using a RAFT agent, sulfur atoms remain at the polymer end in the form of dithioester or trithiocarbonate groups originating from the RAFT agent. When the polymer is used as a base polymer in a resist composition, sulfur atoms at the end incur side reactions which can cause coloration or degradation of storage stability of the resist composition.

The patent documents cited above refer to the removal of end sulfur atoms formed in the living radical polymerization step from the RAFT agent. However, some problems must be overcome before these methods can be used for the mass-scale manufacture of polymers suited as the base polymer. Reference is made to Example 4 of Patent Document 3, for instance. After the step of adding a thiol compound and removing sulfur-containing end groups originating from the RAFT agent, the polymer has an end conversion rate which is as low as 63%. Since none of Examples of Patent Document 3 show an end removal rate in excess of 90%, the method is not regarded satisfactory. In Example 1 of Patent Document 4, a thermal radical generator is added in an amount which is 10 times greater than the amount of the RAFT agent used in the polymerization step, in order to reduce thiocarbonylthio end groups originating from the RAFT agent. In none of Examples, sulfur-containing end groups are completely removed. In this situation, even when the polymer is purified by re-precipitation, the sulfur-containing end groups which invite concern about stability are left behind. There is a risk that coloring and decomposition occur with the lapse of time.

It is believed that the end group can be converted by light irradiation in the presence of a hydrogen atom donor. This method lacks convenience and reaction uniformity in the large-scale industrial manufacture. The light irradiation method is then difficult to produce a RAFT polymer having an end conversion rate of at least 98% in an industrial scale, specifically in an amount of 1 kg or more. A further problem of the light irradiation method is that if unreacted monomers and low-molecular-weight fractions after the polymerization reaction are left behind after the end treatment and even after the purification, they can be a cause to development defects when the polymer is used as a base polymer in a resist composition. A polymer having a high conversion rate of RAFT end and low contents of residual monomers and low-molecular-weight fractions is thus desired.

As mentioned above, unreacted monomers and low-molecular-weight fractions can be a risk to development defects. In view of the time scale of manufacture, a simple method including a smaller number of steps is preferred for polymer synthesis. With respect to the removal of unreacted monomers and low-molecular-weight fractions, the removal by re-precipitation is desirable rather than the removal by liquid-liquid fractionation. It is thus recommended that unreacted monomers, which are left after post-polymerization end treatment and are less soluble in organic solvents, are converted to oligomers that can be removed via crystallization.

As discussed above, the prior art methods are difficult to prepare a resist composition comprising a base polymer that meets the demand in mass-scale device fabrication by the ArF immersion lithography. In particular, the base polymer preparation method which meets both narrowing of polymer dispersity and removal of sulfur-containing end groups has not been established.

### CITATION LIST

Patent Document 1: JP-A 2020-070399
Patent Document 2: JP-A 2005-156725
Patent Document 3: WO 2019/189276
Patent Document 4: WO 2012/165473
Patent Document 5: JP-A 2015-505881

### SUMMARY OF THE INVENTION

An object of the invention is to provide a method for preparing a polymer by living radical polymerization using a RAFT agent, in which the polymer has a narrow dispersity, sulfur-containing end groups originating from the RAFT agent are removed in a simple and efficient way, and unreacted monomers and low degree-of-polymerization fractions having low solvent solubility are removed by purification. Another object is to provide a chemically amplified resist composition comprising the polymer, and a pattern forming process using the chemically amplified resist composition.

The inventors have found that a polymer having a narrow dispersity and a minimal content of residual monomers is obtained by a method based on living radical polymerization using a RAFT agent and being able to remove sulfur-containing end groups originating from the RAFT agent in a simple and efficient way, and that a chemically amplified resist composition comprising the polymer as a base polymer has improved lithography performance and forms patterns of satisfactory profile. The invention is predicated on this finding.

In one aspect, the invention provides a method for preparing a polymer comprising repeat units adapted to be decomposed under the action of acid and free of repeat units adapted to function as a photoacid generator, the method comprising the steps of
(1) polymerizing starting monomers including a monomer having the formula (A1) in a solution through living radical polymerization using a radical initiator and a reversible addition-fragmentation chain transfer agent (RAFT agent), to form a polymer P-1 having an end structure selected from structures having the formulae (X-1) and (X-2) originating from the RAFT agent at the end of the backbone,
(2) adding a radical generator and a thiol compound to the solution containing polymer P-1, and heating the solution to convert the end structure to hydrogen to form a polymer P, and
(3) mixing the solution containing polymer P with a poor solvent and allowing polymer P to precipitate as solids for purification.

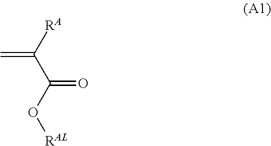

Herein R^{A} is hydrogen or methyl,
R^{AL} is an acid labile structure-containing group having any one of the following formulae (a-1) to (a-10):

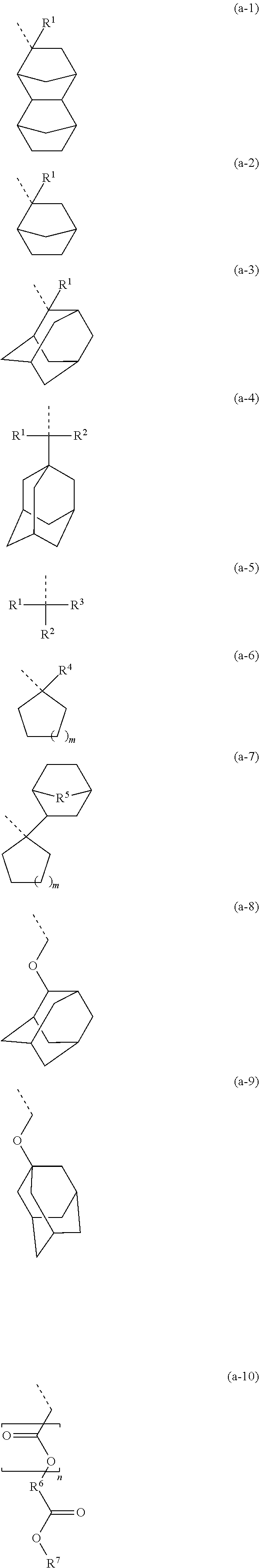

wherein R¹ and R² are each independently a C₁-C₄ alkyl group,
R³ is a C₁-C₄ alkyl group, cyclopentyl, cyclohexyl, oxanorbornyl or thianorbornyl group, some —CH₂— in the cyclopentyl and cyclohexyl groups may be replaced by —O— or —S—,
R⁴ is a C₁-C₄ alkyl group, cyclopentyl group, or cyclohexyl group,
R⁵ is —O—, —S—, methanediyl or ethane-1,2-diyl,
R⁶ is a C₃-C₄₀ divalent alicyclic group which may contain at least one atom selected from oxygen and sulfur,
R⁷ is an acid labile group having the formula (a-3), (a-5) or (a-6),
m is 1 or 2, n is 0 or 1, the broken line designates a point of attachment.

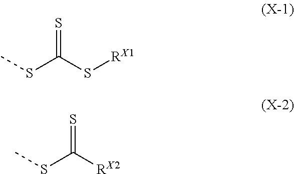

Herein R^{X1} and R^{X2} are each independently a C₂-C₂₀ saturated hydrocarbyl group which may contain a heteroatom, C₇-C₂₀ aralkyl group or C₆-C₂₀ aryl group, the broken line designates a point of attachment to the carbon atom in the backbone.

In a preferred embodiment, the starting monomers further include at least one monomer selected from a monomer having the formula (A2), a monomer having the formula (A3), and a monomer having the formula (A4).

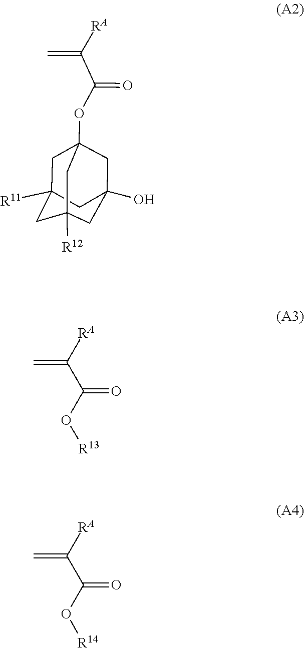

Herein R^{A} is hydrogen or methyl,
R¹¹ and R¹² are each independently hydrogen or hydroxy,
R¹³ is a lactone structure-containing substituent or sultone structure-containing monovalent substituent, and
R¹⁴ is hydrogen, a C₁-C₁₅ fluorinated hydrocarbyl group or C₁-C₁₅ monovalent fluoroalcohol-containing substituent.

In a preferred embodiment, the starting monomers do not include a conjugated monomer.

In a preferred embodiment, the thiol compound is a compound having the formula (SH-1) or (SH-2).

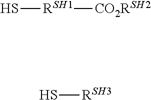

Herein R^{SH1} is a C₁-C₃ hydrocarbylene group,
R^{SH2} is a C₁-C₈ aliphatic hydrocarbyl group which may contain a heteroatom, C₇-C₁₈ aralkyl group or C₆-C₁₈ aryl group, and
R^{SH3} is a C₂-C₂₀ saturated hydrocarbyl group which may contain a heteroatom, C₇-C₁₈ aralkyl group or C₆-C₁₈ aryl group.

Preferably, in step (2), the rate of conversion of the end structure originating from the RAFT agent to hydrogen is at least 98%.

Also preferably, in step (1), the amount of the radical initiator charged is 0.5 to 5 moles and the amount of the RAFT agent charged is 0.5 to 20 moles per 100 moles of all the monomers charged.

In a preferred embodiment, the amounts of monomers remaining at the end of step (2) are less than 0.05% by weight based on the total weight of the monomers charged.

In another aspect, the invention provides a method for preparing a chemically amplified resist composition, comprising the steps of dissolving a raw material comprising a base polymer containing the polymer obtained from the method defined above and a photoacid generator in an organic solvent and mixing them.

In a preferred embodiment, the photoacid generator contains at least one compound selected from a compound having the formula (B1), a compound having the formula (B2), and a compound having the formula (B3).

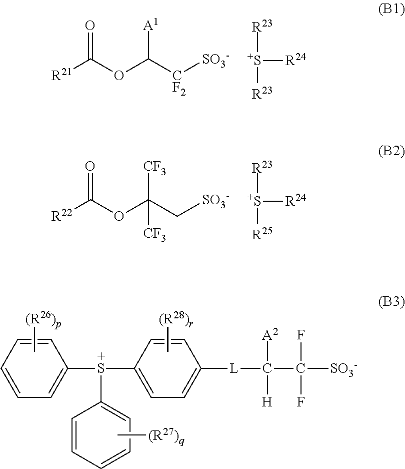

Herein A¹ is hydrogen or trifluoromethyl,
R²¹ and R²² are each independently a C₁-C₃₅ hydrocarbyl group which may contain oxygen, a C₂-C₃₅ monovalent nitrogen-containing heterocyclic group, or a group having the formula (b):

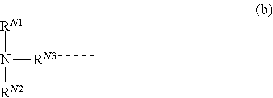

wherein R^{N1} and R^{N2} are each independently hydrogen or a C₁-C₂₀ hydrocarbyl group which may contain a heteroatom, R^{N1} and R^{N2} may bond together to form a ring with the nitrogen atom to which they are attached, R^{N3} is a C₁-C₂₀ hydrocarbylene group which may contain a heteroatom, the broken line designates a point of attachment,
R²³, R²⁴ and R²⁵ are each independently halogen or a C₁-C₃₀ hydrocarbyl group which may contain a heteroatom, any two of R²³, R²⁴ and R²⁵ may bond together to form a ring with the sulfur atom to which they are attached,
R²⁶, R²⁷ and R²⁸ are each independently halogen, a C₁-C₃₀ hydrocarbyl group which may contain a heteroatom, or a C₁-C₂₀ hydrocarbyloxy group which may contain a heteroatom,
p and q are each independently 0, 1, 2, 3, 4 or 5, r is 0, 1, 2, 3 or 4, and
L is a single bond, ether bond, or a C₁-C₂₀ hydrocarbylene group which may contain a heteroatom.

In a preferred embodiment, the raw material further contains a fluorinated polymer comprising repeat units of at least one type selected from repeat units having the formula (D1), repeat units having the formula (D2), and repeat units having the formula (D3).

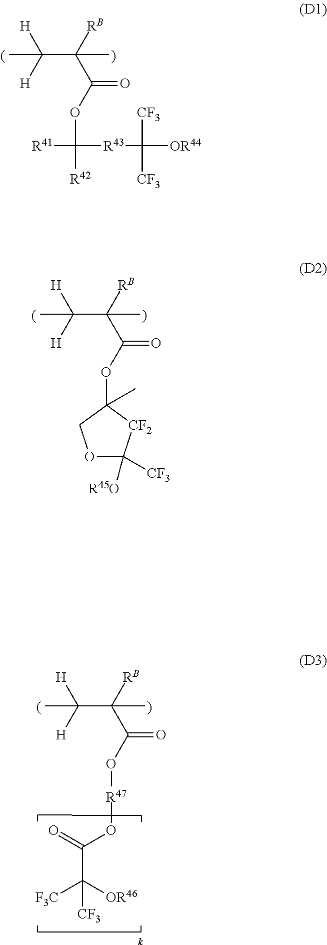

Herein R^{B} is hydrogen or methyl,
R⁴¹ and R⁴² are each independently hydrogen or a C₁-C₁₀ hydrocarbyl group,
R⁴³ is a single bond or a C₁-C₅ straight or branched hydrocarbylene group,
R⁴⁴, R⁴⁵ and R⁴⁶ are each independently hydrogen, a C₁-C₁₅ hydrocarbyl group, C₁-C₁₅ fluorinated hydrocarbyl group, C₂-C₁₅ acyl group, or acid labile group; when R⁴⁴, R⁴⁵ and R⁴⁶ are hydrocarbyl or fluorinated hydrocarbyl groups, some —CH₂— in these groups may be replaced by an ether bond or carbonyl moiety,
R⁴⁷ is a C₁-C₂₀ (k+1)-valent hydrocarbon group or C₁-C₂₀ (k+1)-valent fluorinated hydrocarbon group, and
k is 1, 2 or 3.

In a preferred embodiment, the raw material further contains a quencher and/or a surfactant.

In a further aspect, the invention provides a pattern forming process comprising the steps of applying the chemically amplified resist composition obtained from the method defined above onto a substrate to form a resist film thereon, exposing the resist film to high-energy radiation, and developing the exposed resist film in a developer.

Most often, the high-energy radiation is ArF excimer laser.

In a still further aspect, the invention provides a polymer obtained from living radical polymerization using a RAFT agent, wherein
said polymer comprises repeat units adapted to be decomposed under the action of acid and derived from a monomer having the formula (A1),
at least 98% of the end structure in the polymer, selected from structures having the formulae (X-1) and (X-2) originating from the RAFT agent has been converted to hydrogen,
the amount of the monomer having formula (A1) remaining in the polymer is up to 0.05% by weight,
the polymer has a dispersity of up to 1.50.

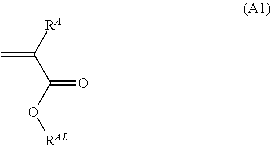

Herein R^{A} is hydrogen or methyl, and R^{AL} is an acid labile structure-containing group having any one of the formulae (a-1) to (a-10):

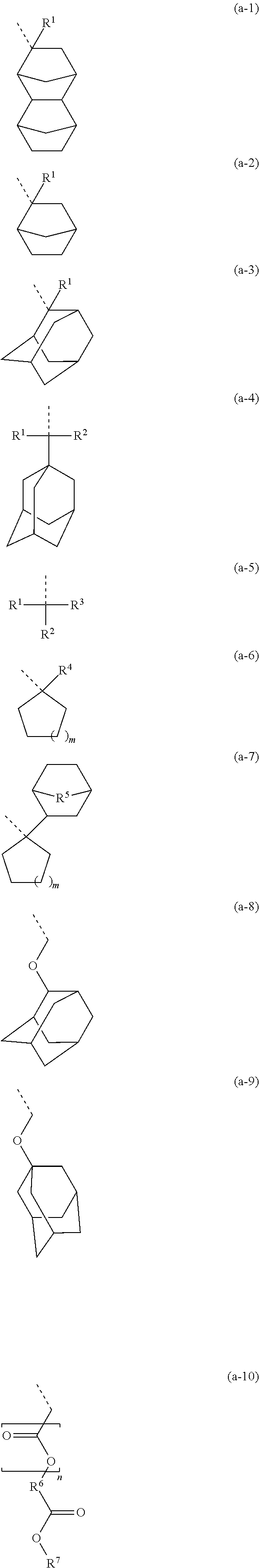

wherein R¹ and R² are each independently a C₁-C₄ alkyl group,
R³ is a C₁-C₄ alkyl group, cyclopentyl group which may contain a heteroatom, cyclohexyl, oxanorbornyl or thianorbornyl group,
R⁴ is a C₁-C₄ alkyl group, cyclopentyl or cyclohexyl group,
R⁵ is —O—, —S—, methanediyl or ethane-1,2-diyl,
R⁶ is a C₃-C₄₀ divalent alicyclic group which may contain at least one atom selected from oxygen and sulfur,
R⁷ is an acid labile group having the formula (a-3), (a-5) or (a-6),
m is 1 or 2, n is 0 or 1,
the broken line designates a point of attachment,

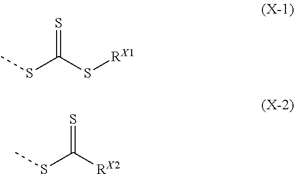

wherein R^{X1} and R^{X2} are each independently a C₂-C₂₀ saturated hydrocarbyl group which may contain a heteroatom, C₇-C₂₀ aralkyl group, or C₆-C₂₀ aryl group, the broken line designates a point of attachment to the carbon atom in the backbone.

The polymer may further comprise repeat units derived from at least one monomer selected from a monomer having the formula (A2), a monomer having the formula (A3), and a monomer having the formula (A4), the amount of each monomer remaining in the polymer is up to 0.05% by weight,

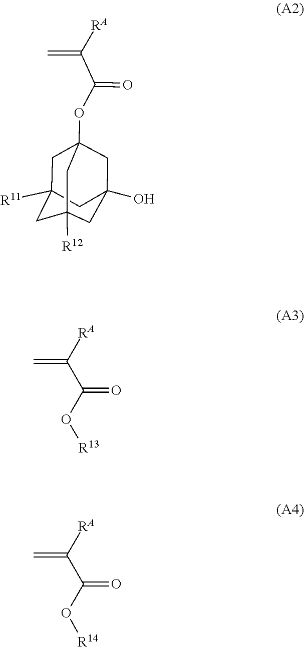

wherein R^{A} is hydrogen or methyl,
R¹¹ and R¹² are each independently hydrogen or hydroxy,
R¹³ is a lactone structure-containing substituent or sultone structure-containing monovalent substituent, and
R¹⁴ is hydrogen, a C₁-C₁₅ fluorinated hydrocarbyl group or C₁-C₁₅ monovalent fluoroalcohol-containing substituent.

Also contemplated herein is a chemically amplified resist composition comprising a base polymer containing the polymer defined herein and a photoacid generator. The resist composition is suited for use in ArF lithography or ArF immersion lithography.

### Advantageous Effects of the Invention

According to the polymer preparation method of the invention, a polymer having a narrow dispersity and low contents of residual monomers, from which sulfur-containing end groups originating from the RAFT agent are removed in an efficient way, is obtained. From a chemically amplified resist composition comprising the polymer, a pattern having an improved DOF and MEF can be formed. The resist composition is suitable for the ArF immersion lithography and effective in forming both positive patterns via alkaline development and negative patterns via organic solvent development.

### DESCRIPTION OF THE PREFERRED EMBODIMENT

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, the broken line (---) designates a point of attachment, namely valence bond. As used herein, the term “halogenated” refers to a halogen-substituted or halogen-containing compound or group. For example, “fluorinated” refers to a fluorine-substituted or fluorine-containing compound or group. The terms “group” and “moiety” are interchangeable.

The abbreviations and acronyms have the following meaning.
RAFT agent: reversible addition-fragmentation chain transfer agent
PAG: photoacid generator
Mw: weight average molecular weight
Mn: number average molecular weight
Mw/Mn: molecular weight distribution or dispersity
GPC: gel permeation chromatography
PEB: post-exposure baking
LER: line edge roughness
CDU: critical dimension uniformity
DOF: depth of focus
MEF: mask error factor

It is understood that for some structures represented by chemical formulae, there can exist enantiomers and diastereomers because of the presence of asymmetric carbon atoms. In such a case, a single formula collectively represents all such isomers. The isomers may be used alone or in admixture.

One embodiment of the invention is a method for preparing a polymer comprising repeat units adapted to be decomposed under the action of acid and free of repeat units adapted to function as a photoacid generator. The method comprising the steps of:
(1) polymerizing starting monomers including a monomer having the formula (A1) in a solution through living radical polymerization using a radical initiator and a RAFT agent, to form a polymer P-1 having an end structure selected from structures having the formulae (X-1) and (X-2) originating from the RAFT agent at the end of the backbone,
(2) adding a radical generator and a thiol compound to the solution containing polymer P-1, and heating the solution to convert the end structure to hydrogen to form a polymer P, and
(3) mixing the solution containing polymer P with a poor solvent and allowing polymer P to precipitate as solids for purification.

The living radical polymerization using a radical initiator and a RAFT agent can be performed with reference to the methods described in JP 3639859, JP-A 2006-002096, Patent Documents 2 and 3. When it is desired to prepare a base polymer to be contained in a resist composition for use in the mass-scale fabrication of devices by the ArF immersion lithography, any of the preparation methods proposed in the prior art suffers from many problems as discussed above. Our discovery reveals that the combination of steps (1) to (3) and proper conditions of each step are effective. Now steps (1) to (3) are described in detail.

### [Step (1)]

In step (1), starting monomers including a monomer having the formula (A1), also referred to as monomer A1, are polymerized in a solution through living radical polymerization using a radical initiator and a RAFT agent, to form a polymer P-1 having an end structure selected from structures having the formulae (X-1) and (X-2) originating from the RAFT agent at the end of the backbone.

### <Monomers>

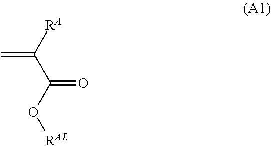

In formula (A1), R^{A} is hydrogen or methyl.

In formula (A1), R^{AL} is an acid labile structure-containing group having any one of the following formulae (a-1) to (a-10).

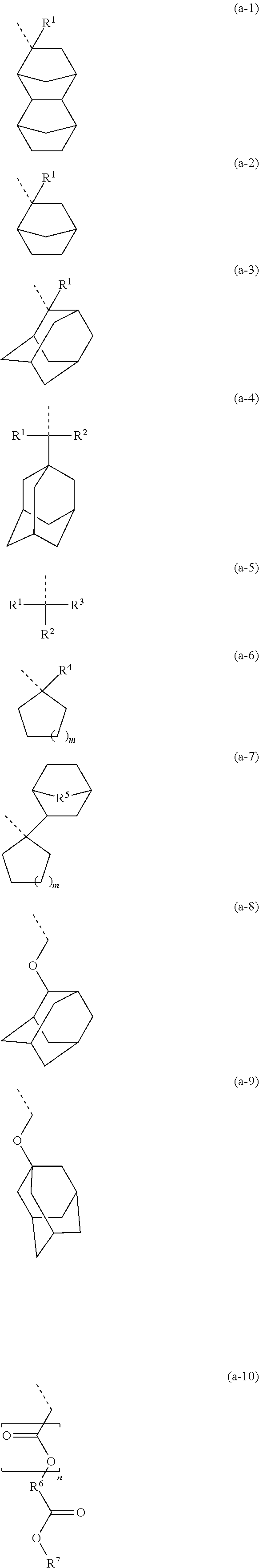

In formulae (a-1) to (a-5), R¹ and R² are each independently a C₁-C₄ alkyl group. Exemplary of the alkyl group are methyl, ethyl, n-propyl, isopropyl, n-butyl, isobutyl, sec-butyl and tert-butyl.

In formula (a-5), R³ is a C₁-C₄ alkyl group, cyclopentyl, cyclohexyl, oxanorbornyl or thianorbornyl group, some —CH₂— in the cyclopentyl and cyclohexyl groups may be replaced by —O— or —S—. Exemplary of the alkyl group are methyl, ethyl, n-propyl, isopropyl, n-butyl, isobutyl, sec-butyl and tert-butyl.

In formula (a-6), R⁴ is a C₁-C₄ alkyl group, cyclopentyl group, or cyclohexyl group. Exemplary of the alkyl group are methyl, ethyl, n-propyl, isopropyl, n-butyl, isobutyl, sec-butyl and tert-butyl. The subscript m is 1 or 2.

In formula (a-7), R⁵ is —O—, —S—, methanediyl or ethane-1,2-diyl. The subscript m is 1 or 2.

In formula (a-10), R⁶ is a C₃-C₄₀ divalent alicyclic group which may contain at least one atom selected from oxygen and sulfur. R⁶ is preferably a C₃-C₄₀ divalent group containing a lactone or sultone ring structure, more preferably a C₆-C₁₂ divalent group containing a lactone or sultone ring structure.

Examples of the divalent group R⁶ are shown below, but not limited thereto.

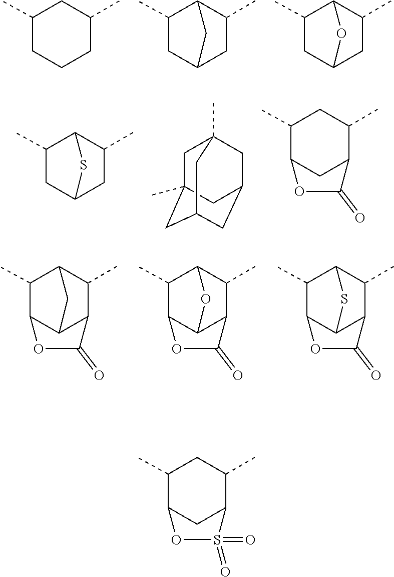

In formula (a-10), R⁷ is an acid labile group having the formula (a-3), (a-5) or (a-6), and n is 0 or 1.

Examples of the acid labile group having formula (a-1) are shown below, but not limited thereto.

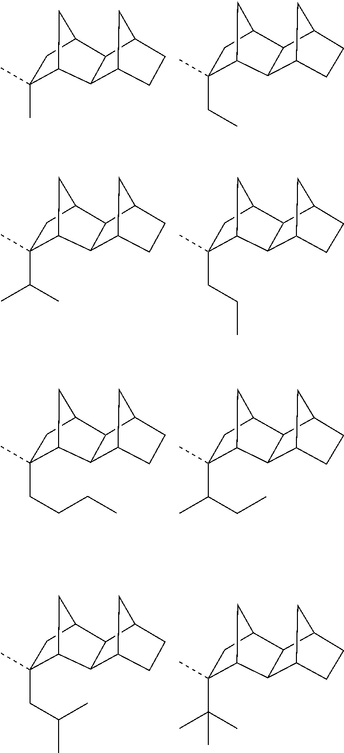

In the group having formula (a-1), there can exist stereoisomers (enantiomers and diastereomers). There may exist either one or both.

Examples of the acid labile group having formula (a-2) are shown below, but not limited thereto.

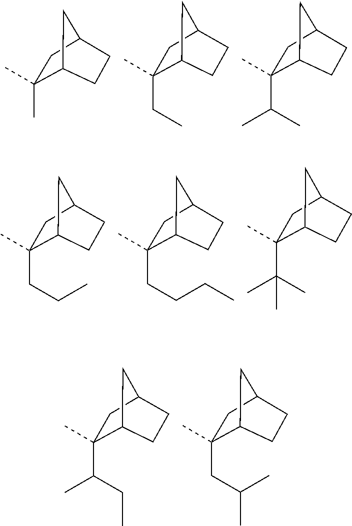

Examples of the acid labile group having formula (a-3) are shown below, but not limited thereto.

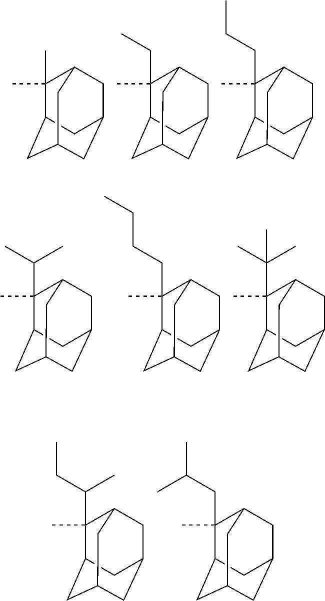

Examples of the acid labile group having formula (a-4) are shown below, but not limited thereto.

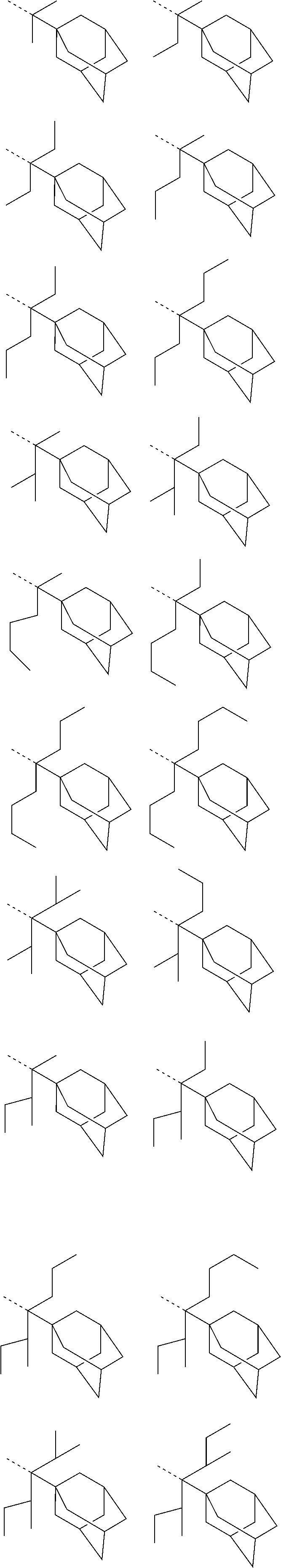

Examples of the acid labile group having formula (a-5) are shown below, but not limited thereto.

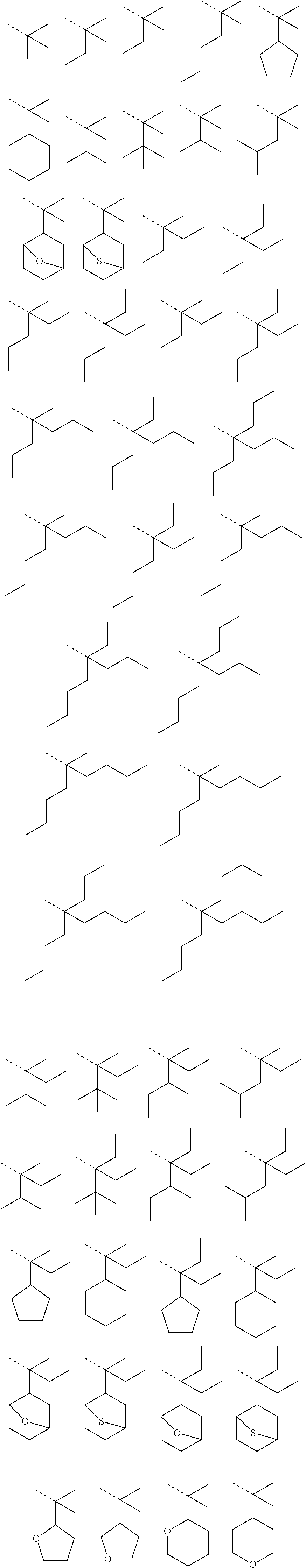

Examples of the acid labile group having formula (a-6) are shown below, but not limited thereto.

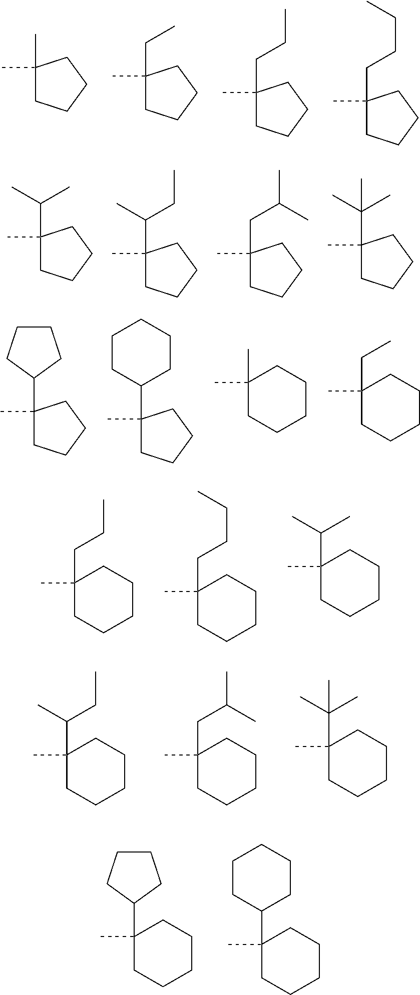

Examples of the acid labile group having formula (a-7) are shown below, but not limited thereto.

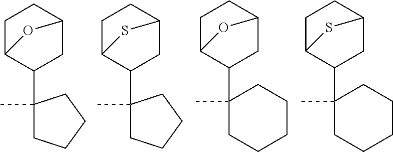

Examples of the acid labile group having formula (a-10) are shown below, but not limited thereto.

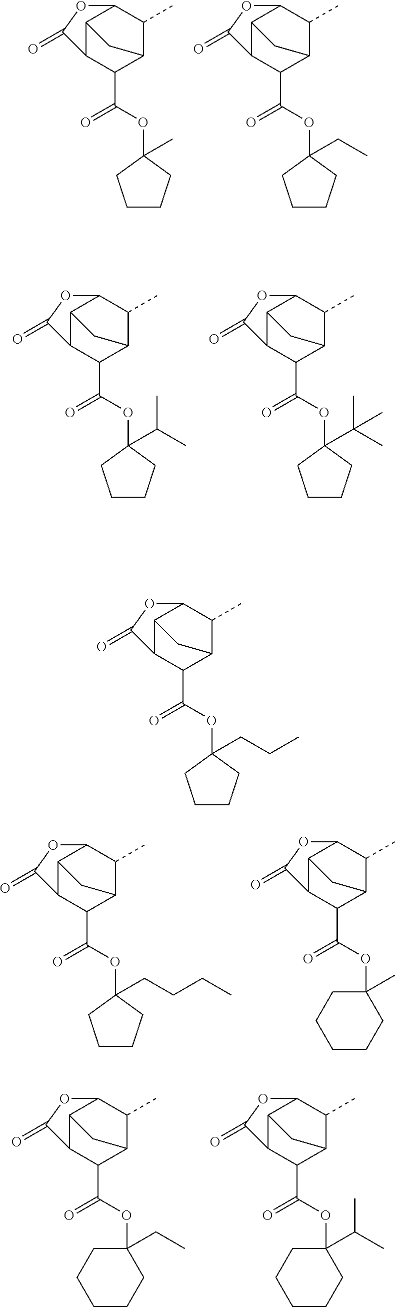

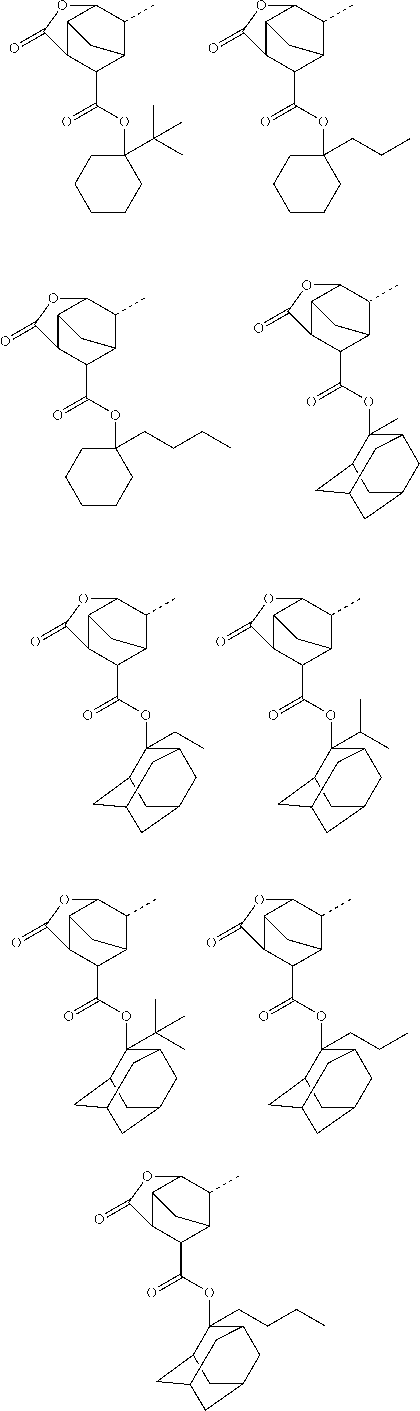

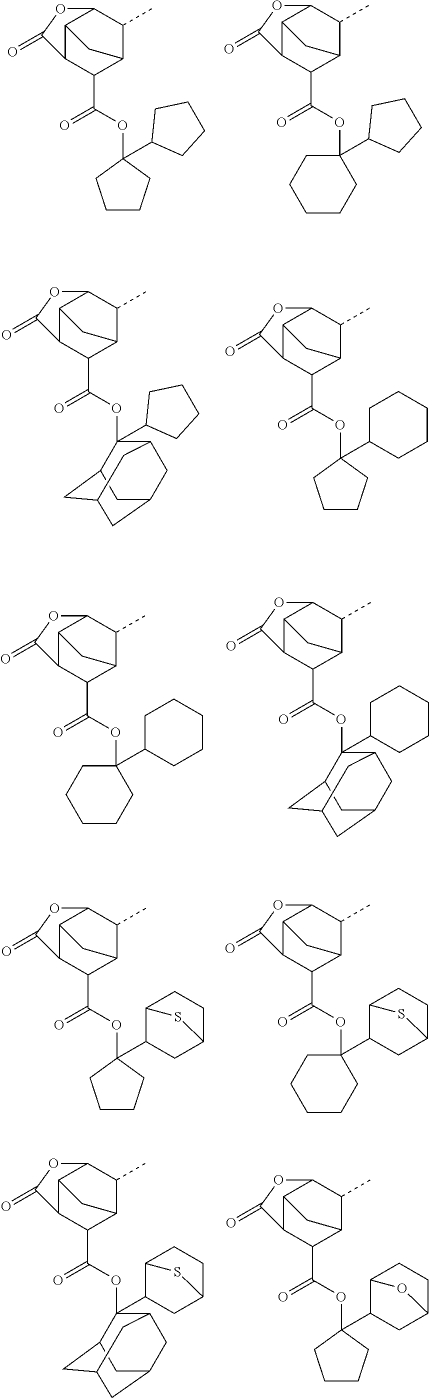

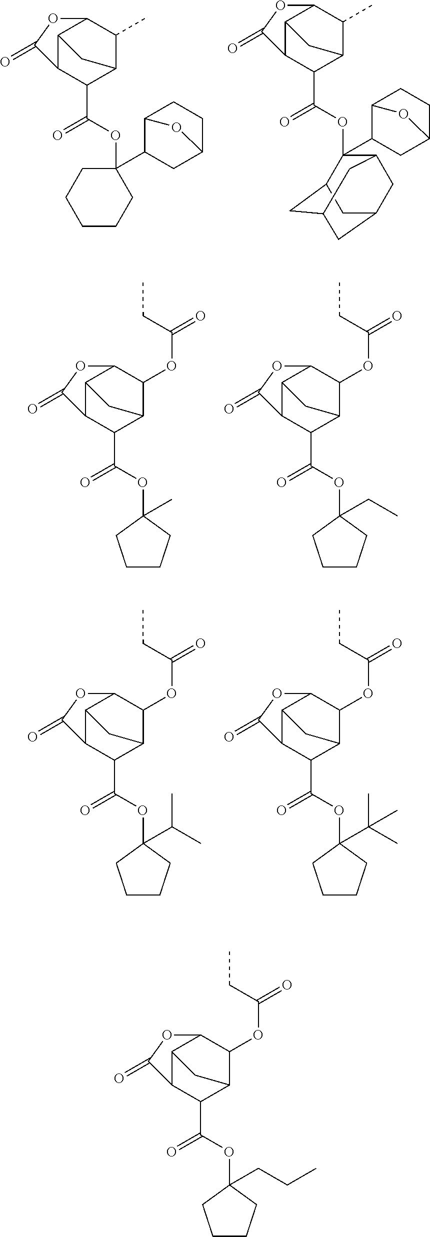

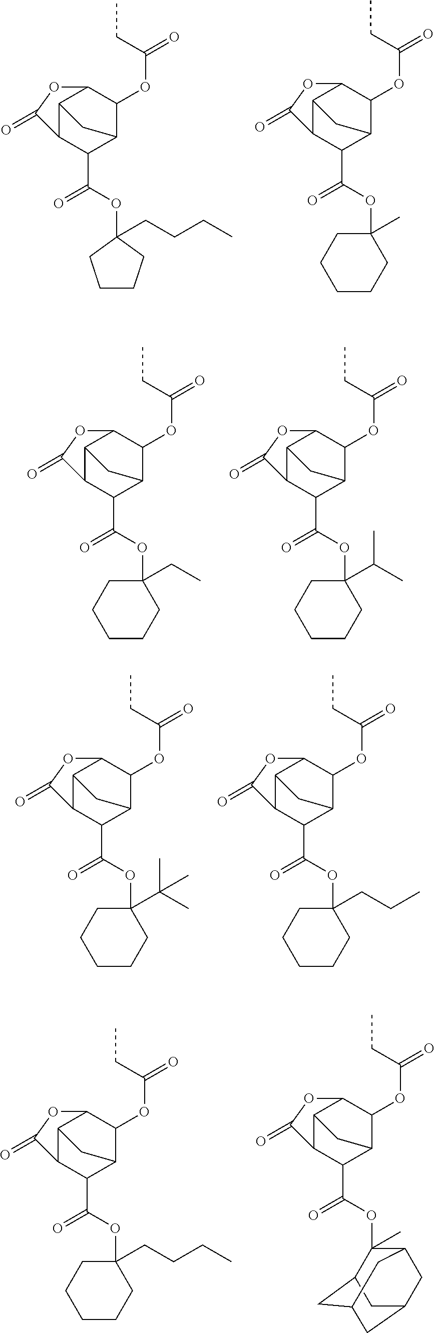

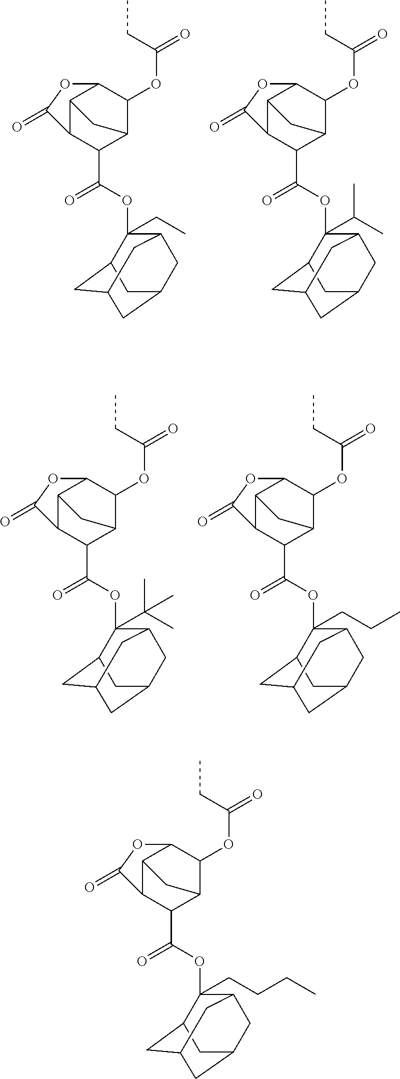

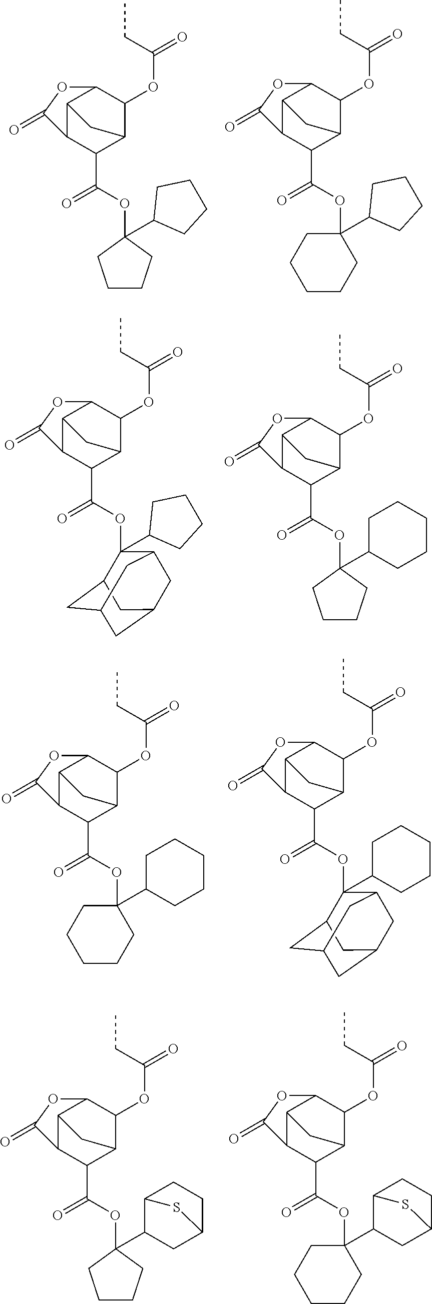

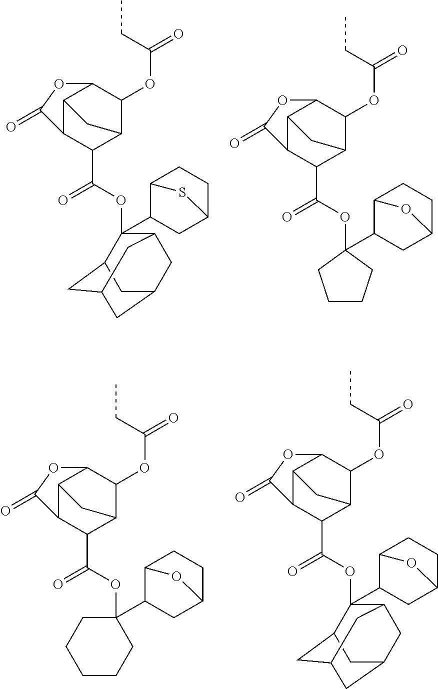

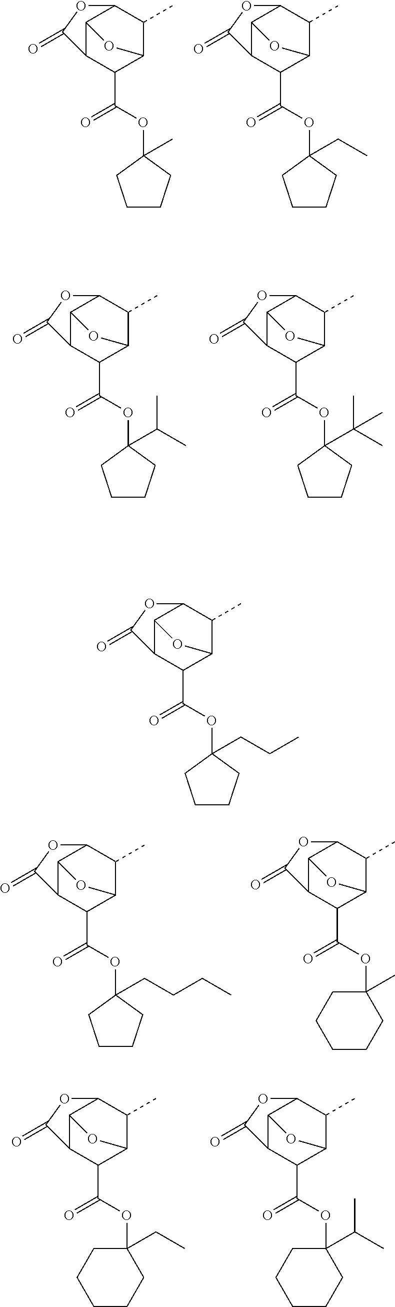

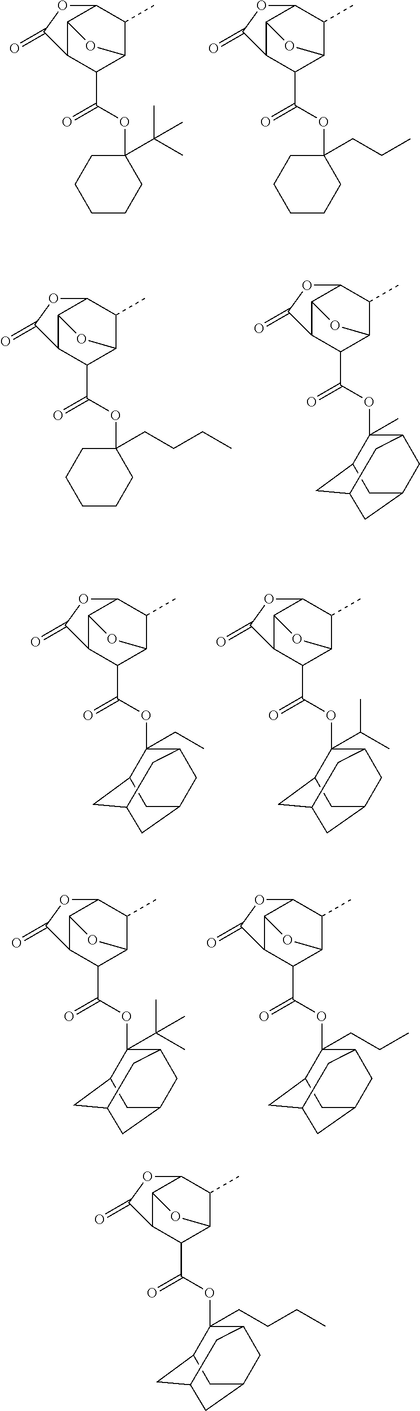

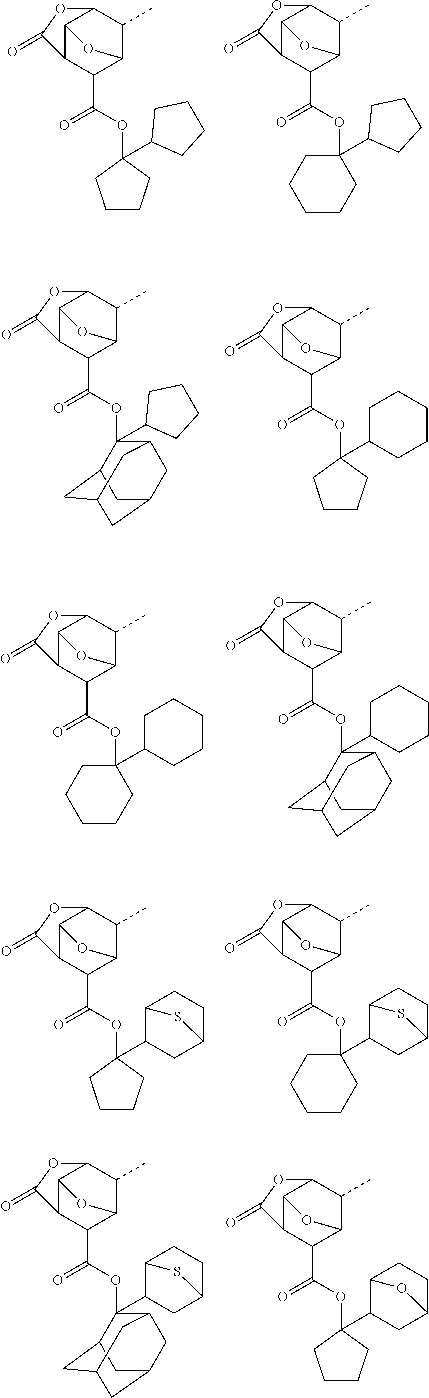

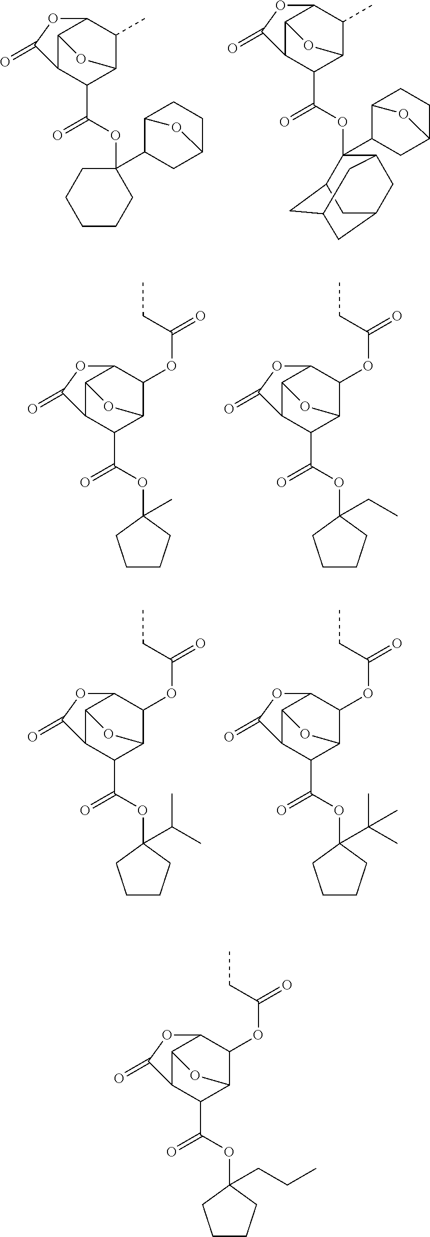

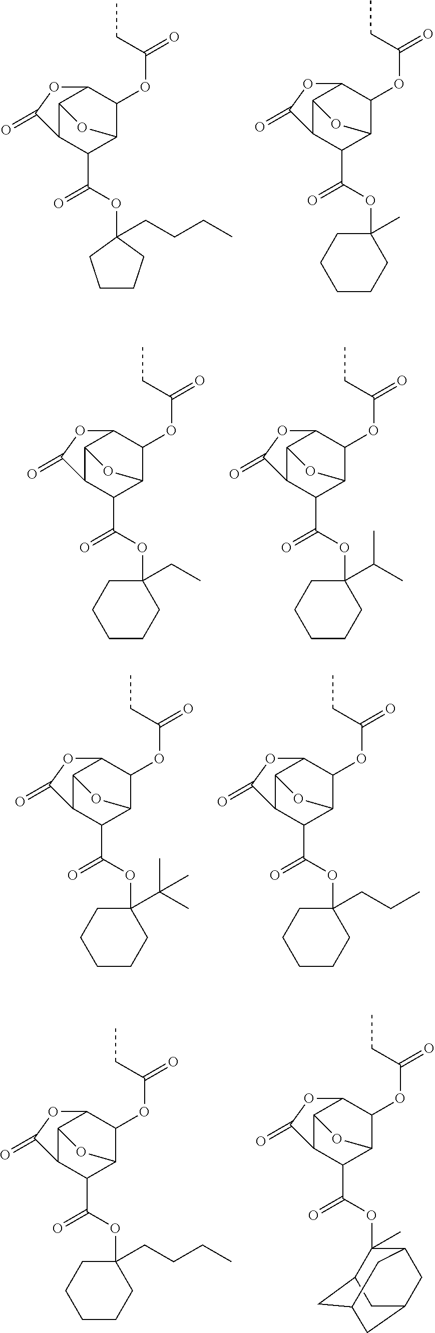

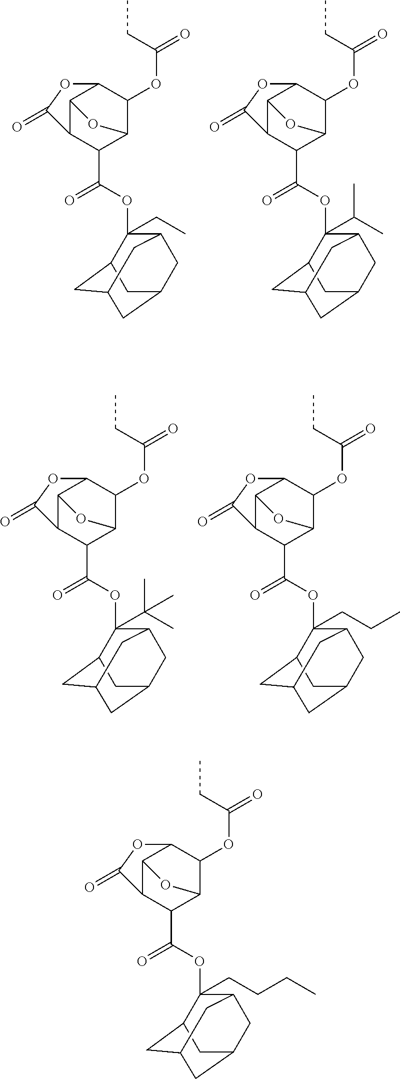

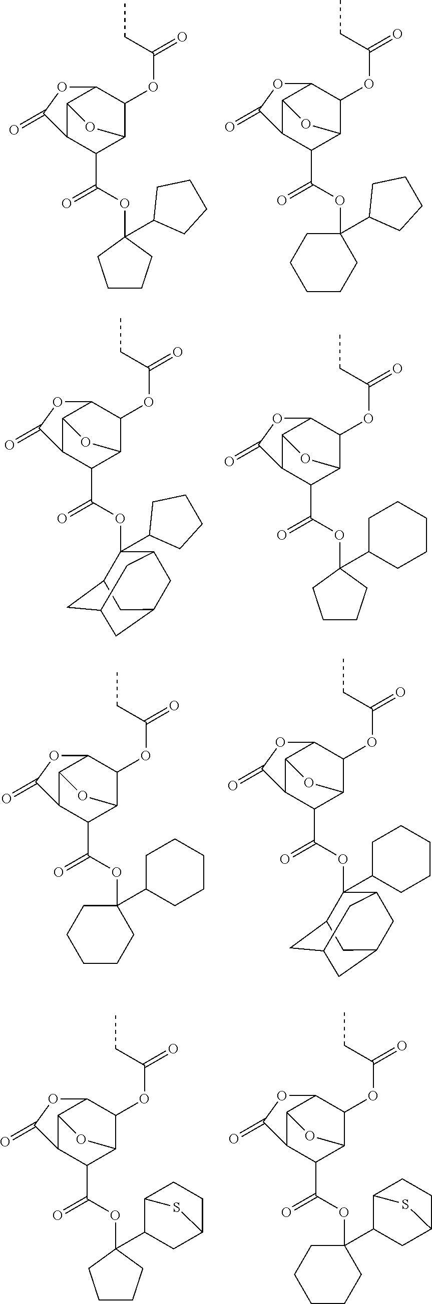

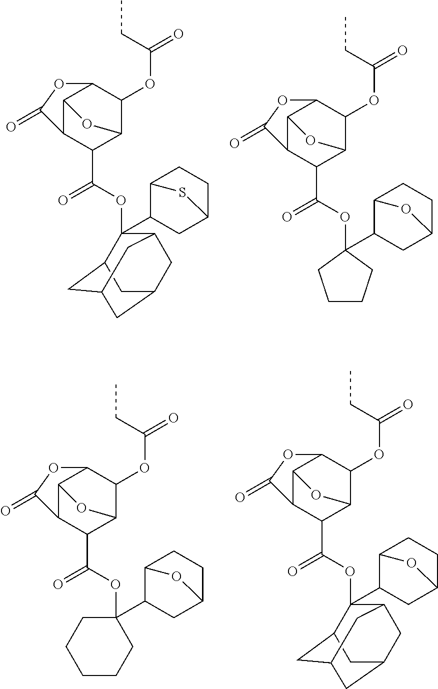

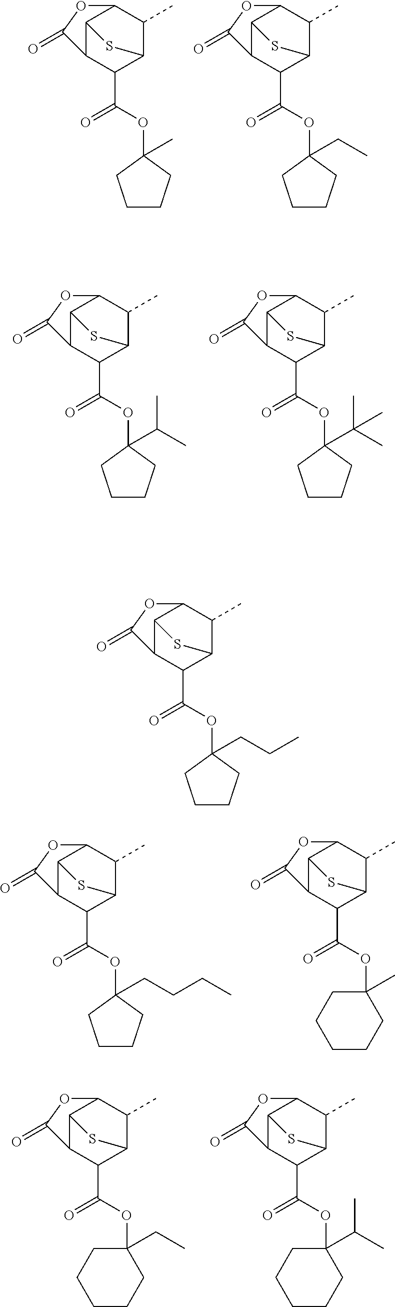

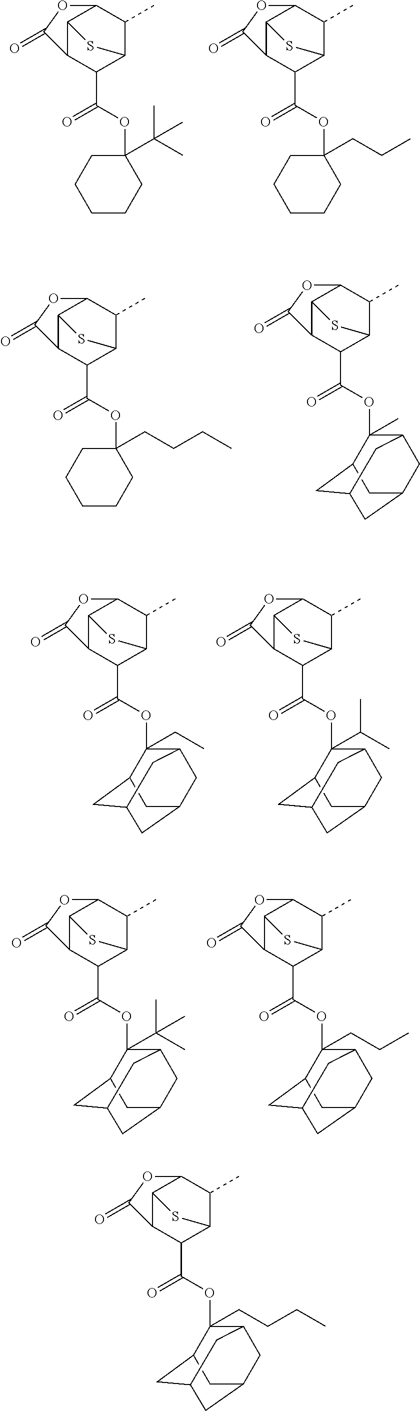

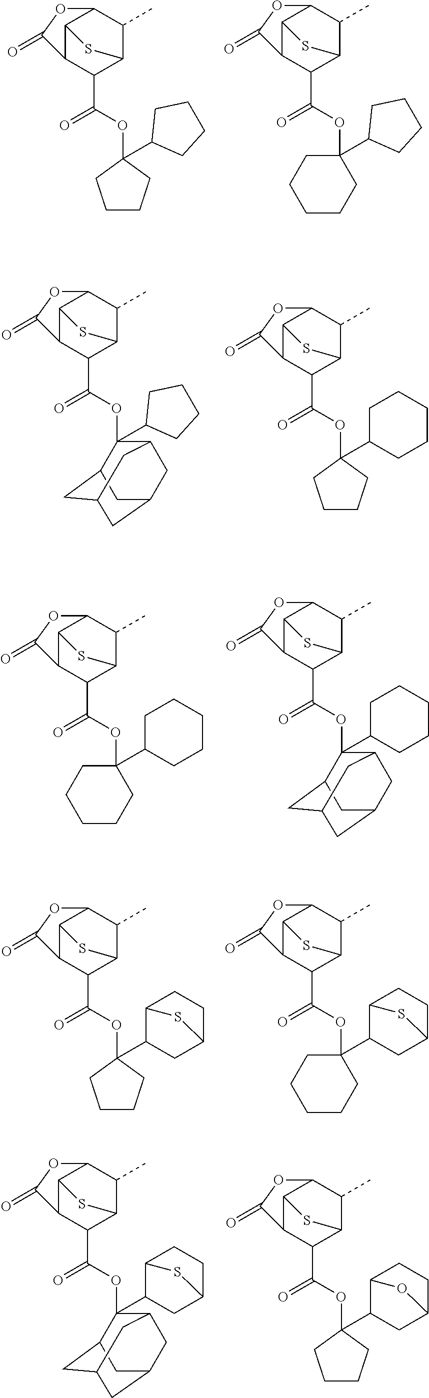

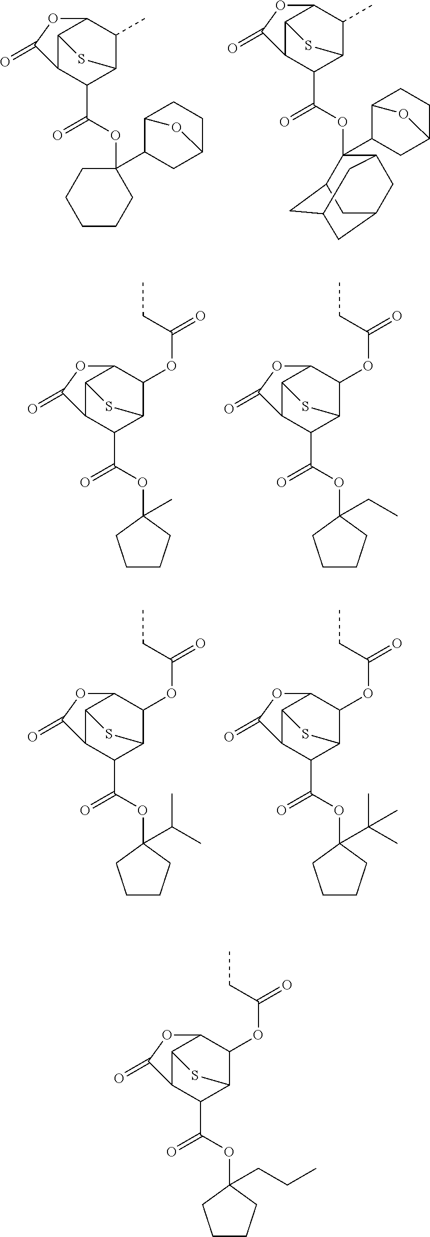

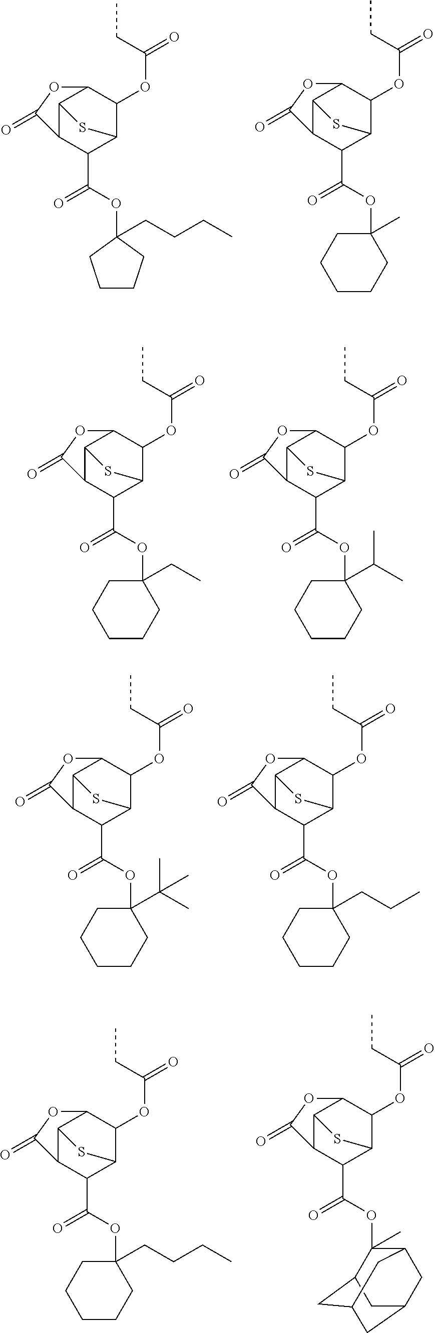

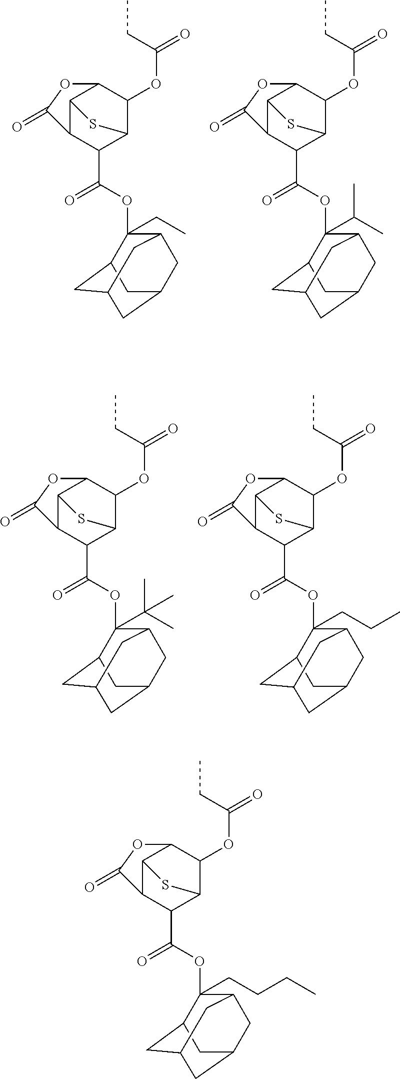

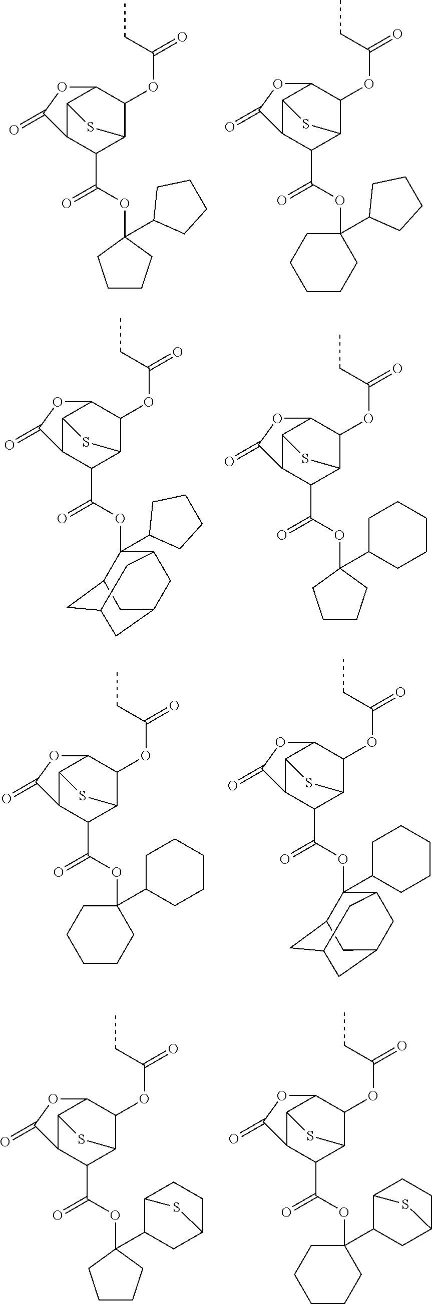

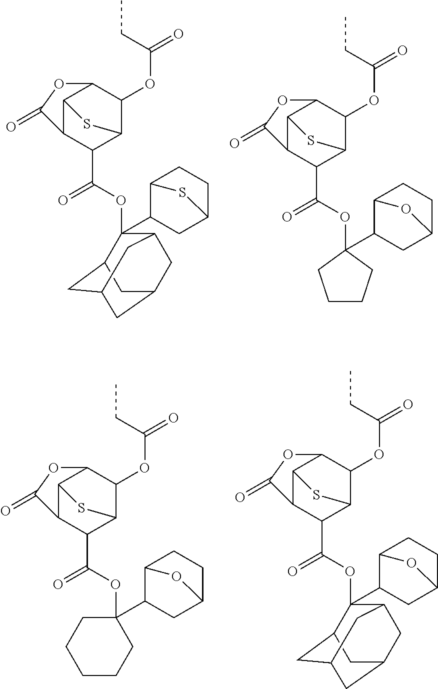

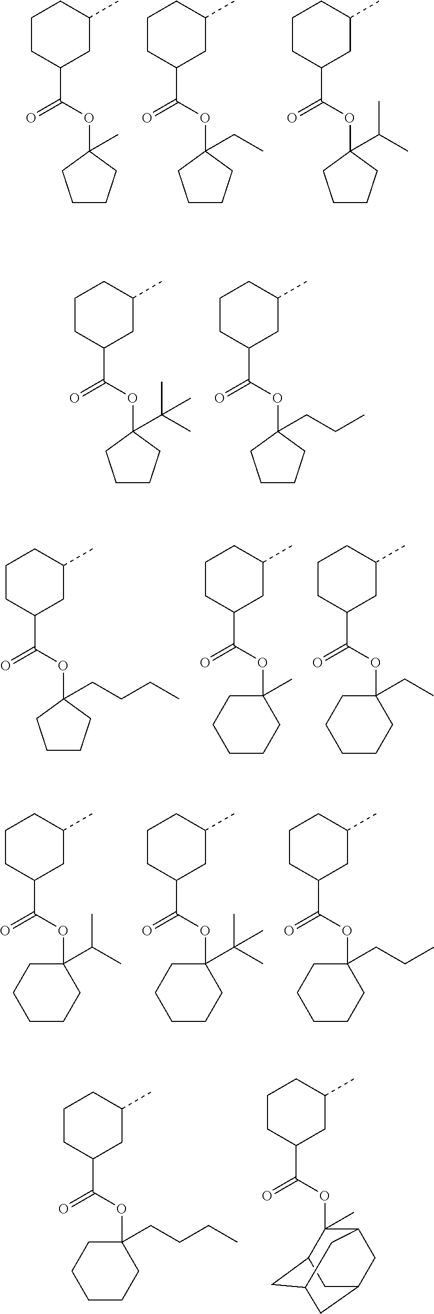

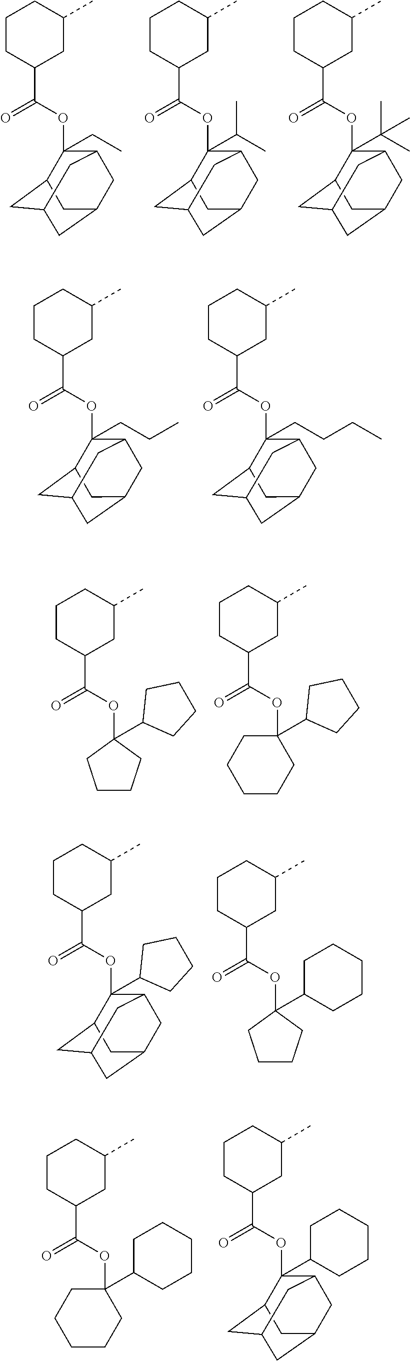

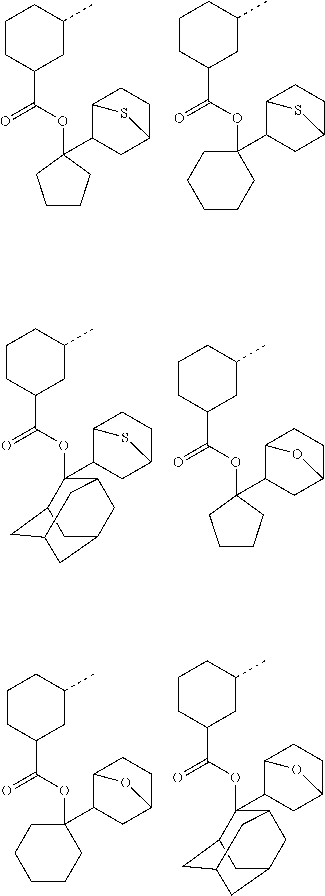

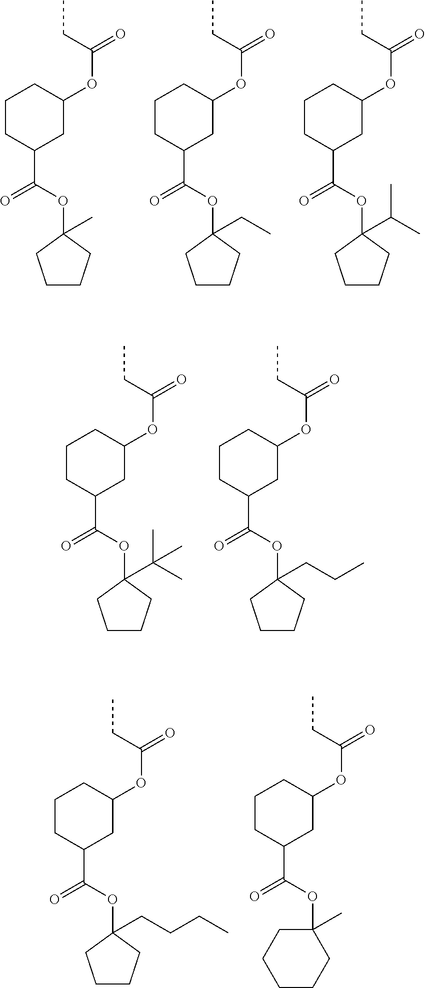

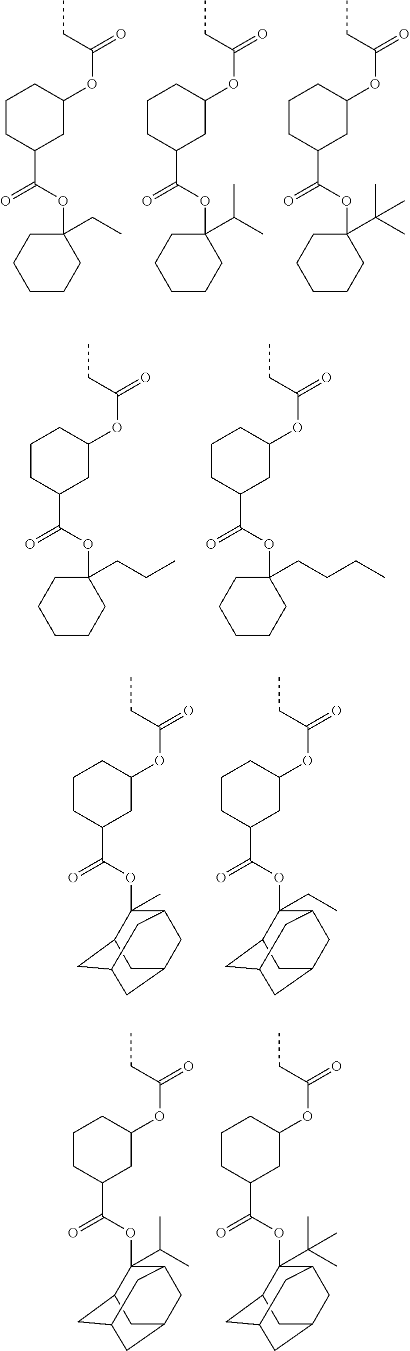

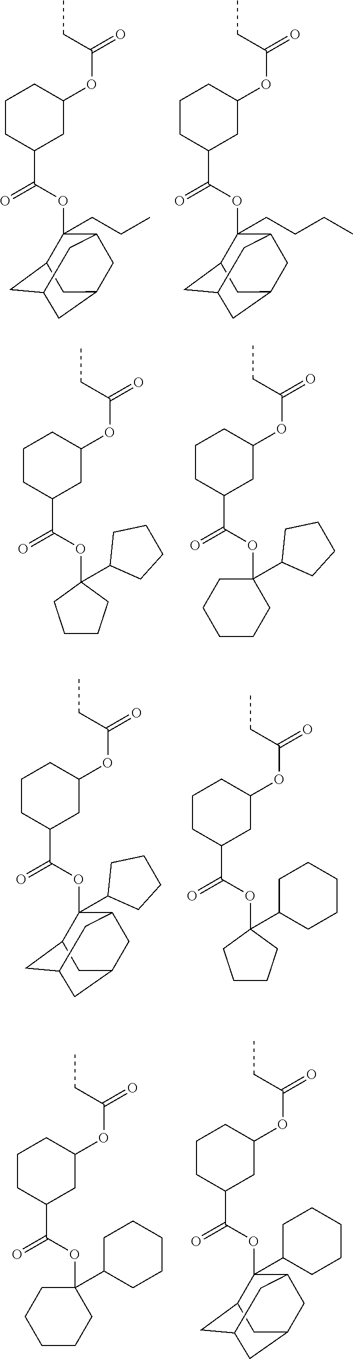

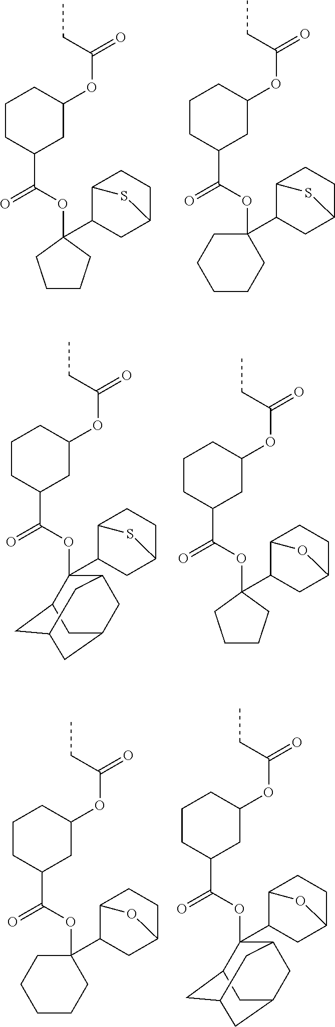

If necessary, the starting monomers may further contain at least one monomer selected from a monomer having the formula (A2), a monomer having the formula (A3), and a monomer having the formula (A4), which are also referred to as monomers A2, A3 and A4, respectively.

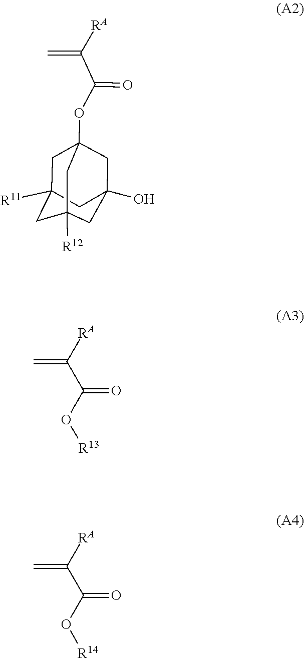

In formulae (A2) to (A4), R^{A} is hydrogen or methyl. R¹¹ and R¹² are each independently hydrogen or hydroxy. R¹³ is a monovalent group containing a lactone or sultone structure. R¹⁴ is hydrogen, a C₁-C₁₅ fluorinated hydrocarbyl group or C₁-C₁₅ monovalent fluoroalcohol-containing group.

Examples of monomer A2 are shown below, but not limited thereto. R^{A} is as defined above.

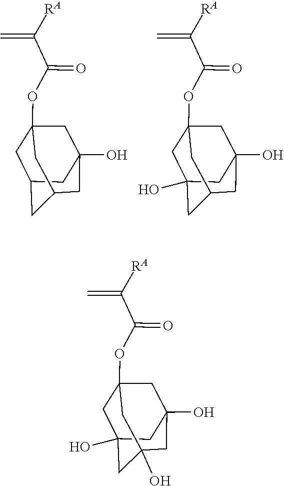

Examples of monomer A3 are shown below, but not limited thereto. R^{A} is as defined above.

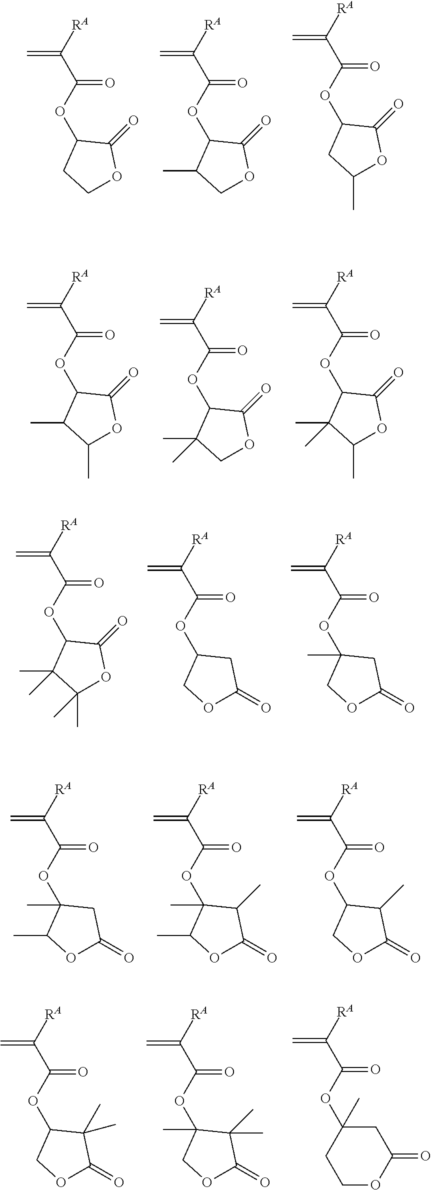

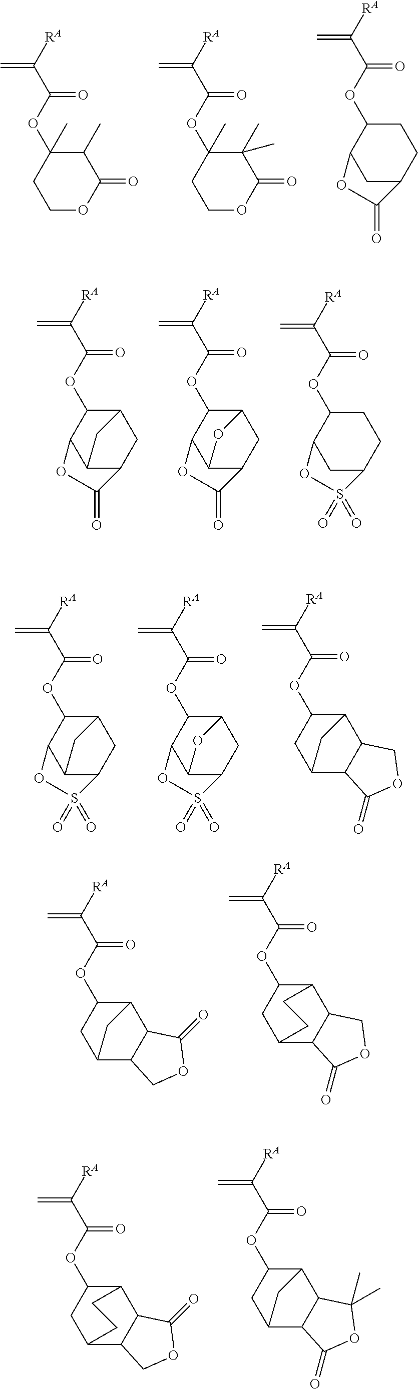

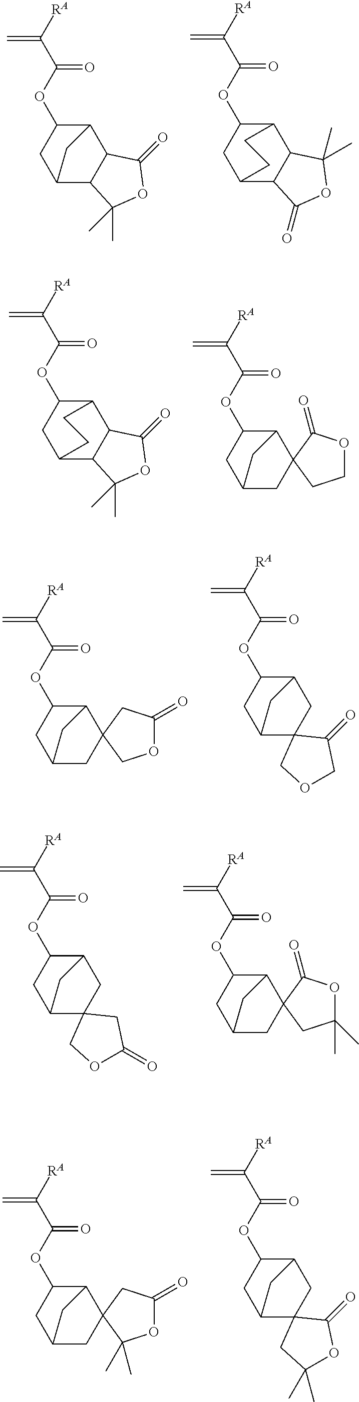

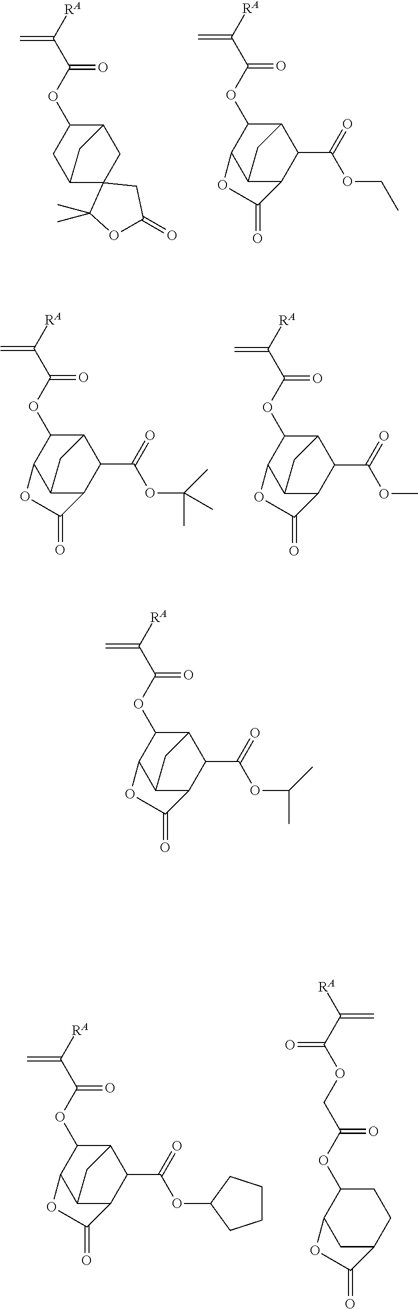

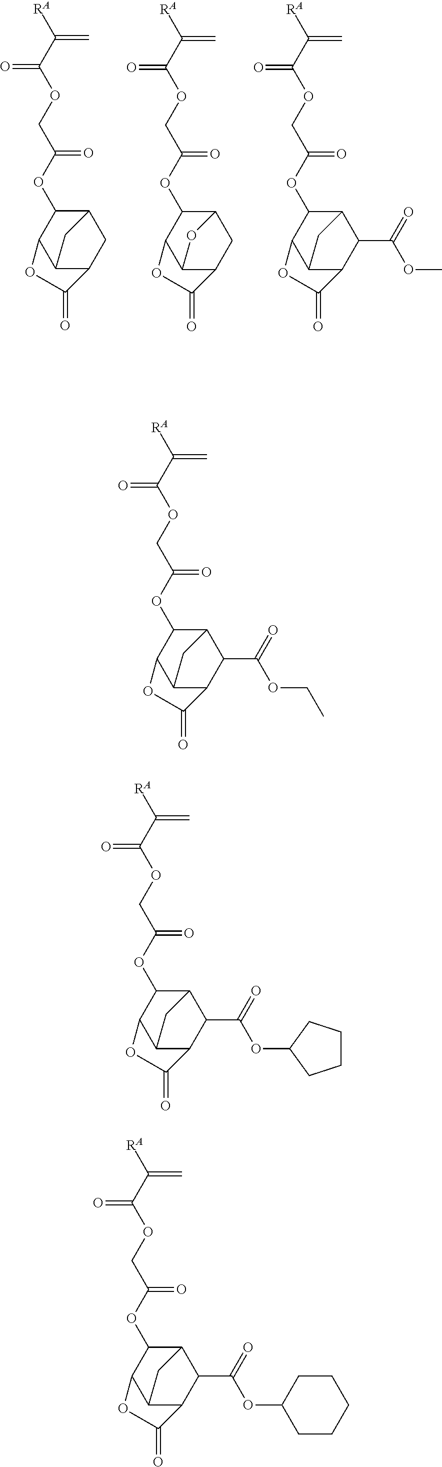

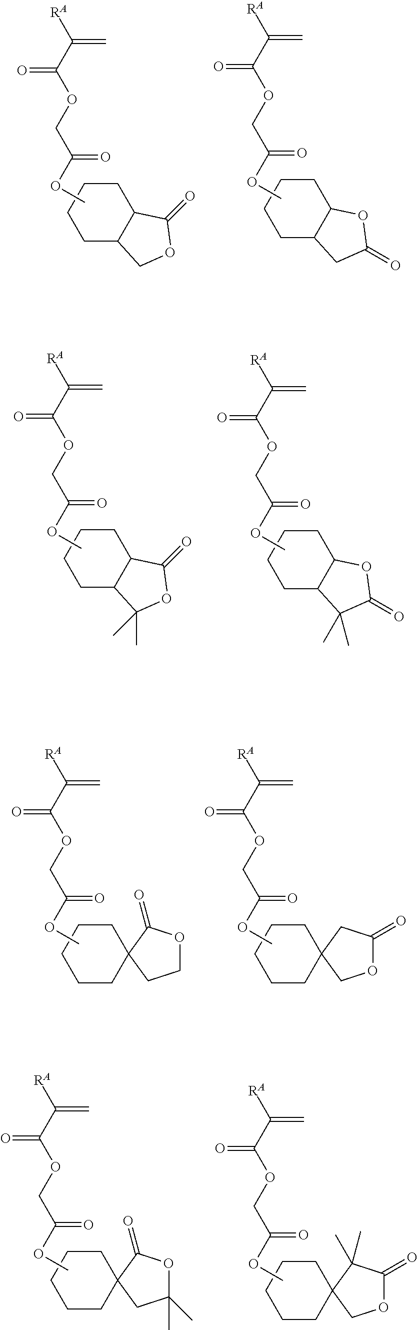

Examples of monomer A4 are shown below, but not limited thereto. R^{A} is as defined above.

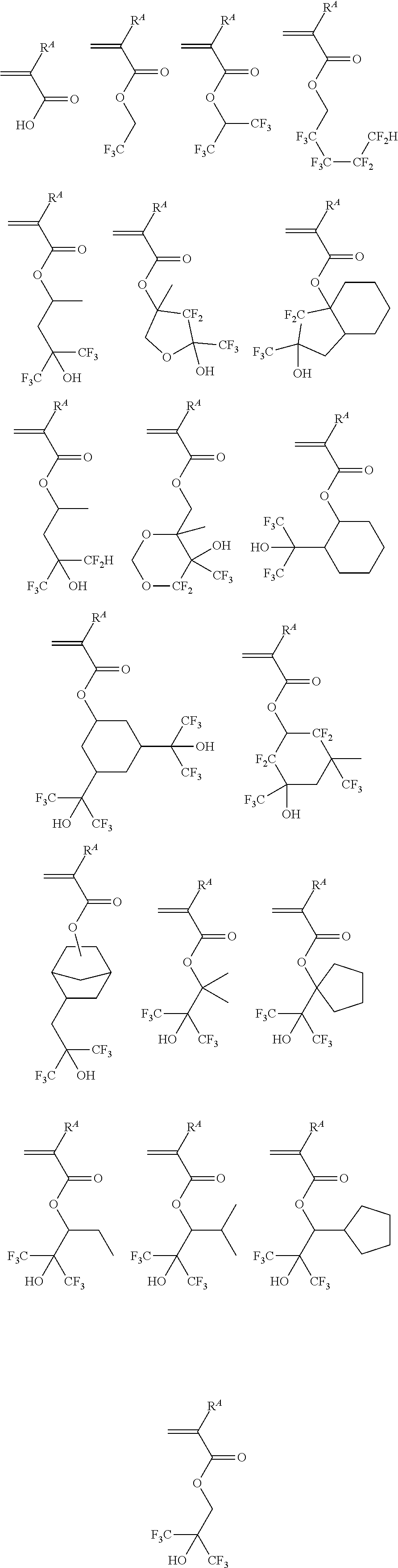

If necessary, the starting material may contain a monomer having a carbon-carbon double bond other than the above-mentioned monomers. Examples of the other monomer include substituted acrylates such as methyl methacrylate, methyl crotonate, dimethyl maleate, and dimethyl itaconate; unsaturated carboxylic acids such as maleic acid, fumaric acid and itaconic acid; cyclic olefins such as norbornene, norbornene derivatives, and tetracyclo[4.4.0.1^{2,5}.17^{7,10}]dodecene derivatives; unsaturated acid anhydrides such as itaconic anhydride; α-methyl-γ-butyrolactone and a-methylstyrene.

In step (1), the proportions of the monomers used preferably fall in the following range (mol %), but are not limited thereto.
(I) 1 to 98 mol %, more preferably 20 to 70 mol %, even more preferably 30 to 60 mol % of monomer A1, based on the overall monomers,
(II) 0 to 50 mol %, more preferably 0 to 20 mol %, even more preferably 0 to 15 mol % of monomer A2, based on the overall monomers,
(III) 1 to 98 mol %, more preferably 20 to 70 mol %, even more preferably 30 to 60 mol % of monomer A3, based on the overall monomers,
(IV) 0 to 20 mol %, more preferably 0 to 10 mol %, even more preferably 0 to 5 mol % of monomer A4, based on the overall monomers, and
(V) 0 to 97 mol %, more preferably 0 to 70 mol %, even more preferably 0 to 50 mol % of one or more monomers other than monomers A1 to A4, based on the overall monomers.

In step (1), monomer A3 is preferably used in admixture of two or more, specifically at least one monomer containing a monocyclic lactone structure and at least one monomer containing a cyclic lactone structure in an alicyclic group obtained by fusing a plurality of cyclic groups. Using a polymer comprising repeat units derived from a plurality of monomers A3, further improvements in DOF and MEF are achieved.

### <RAFT Agent>

The RAFT agent is to introduce a structure having the formula (X-1) or (X-2) at the end of the polymer.

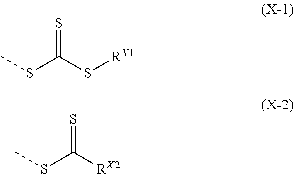

In formulae (X-1) and (X-2), R^{X1} and R^{X2} are each independently a C₂-C₂₀ saturated hydrocarbyl group which may contain a heteroatom, C₇-C₂₀ aralkyl group, or C₆-C₂₀ aryl group. The broken line designates a point of attachment to a carbon atom in the polymer backbone.

The C₂-C₂₀ saturated hydrocarbyl group represented by R^{X1} and R^{X2} may be straight, branched or cyclic. Examples thereof include alkyl groups such as ethyl, n-propyl, isopropyl, n-butyl, tert-butyl, n-pentyl, n-hexyl, n-octyl, n-nonyl, n-decyl, undecyl, dodecyl, n-tetradecyl, n-hexadecyl, n-octadecyl, and n-eicosanyl; and cyclic saturated hydrocarbyl groups such as cyclopentyl, cyclohexyl, cyclooctyl, cyclodecyl, cyclododecyl, 1-adamantyl, 2-adamantyl, 1-methylcyclopentyl, 1-isopropylcyclopentyl, 1-methylcyclohexyl, 1-isopropylcyclohexyl, 1-methyladamantyl and 1-ethyladamantyl.

Examples of the C₇-C₂₀ aralkyl group represented by R^{X1} and R^{X2} include benzyl, phenethyl, 4-methoxybenzyl and 9-anthracenylmethyl. Examples of the C₆-C₂₀ aryl group represented by R^{X1} and R^{X2} include phenyl, naphthyl, 4-methoxyphenyl, 2-anthracenyl and 9-anthracenyl.

Since (meth)acrylate type monomers are used, compounds having the formulae (CTA-1) and (CTA-2) are preferred as the RAFT agent.

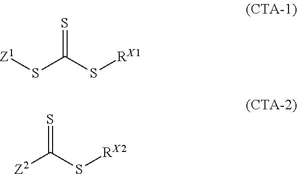

In formulae (CTA-1) and (CTA-2), R^{X1} and R^{X2} are each independently a C₂-C₂₀ saturated hydrocarbyl group which may contain a heteroatom, C₇-C₂₀ aralkyl group, or C₆-C₂₀ aryl group. Z¹ and Z² are each independently a C₃-C₂₀ saturated hydrocarbylthio group, C₇-C₂₀ aralkylthio group, C₅-C₂₀ heterocyclyl group, —N(Z^{A})(Z^{B}), —COOZ^{A}, —OCOZ^{A}, —CON(Z^{A})(Z^{B}), —P(═O)(OZ^{A})₂ or —O—P(═O)(Z^{A})(Z^{B}). Z^{A} and Z^{B} are each independently a C₁-C₂₀ saturated hydrocarbyl group, C₆-C₂₀ aryl group or C₇-C₂₀ aralkyl group. In Z¹ and Z², some or all of the carbon-bonded hydrogen atoms may be substituted by cyano, carboxy or the like, and Z¹ and Z² may contain an acid labile group.

The RAFT agents (CTA-1) and (CTA-2) are trithiocarbonate and dithioester compounds, respectively. Examples of the trithiocarbonate compound include 2-cyano-2-propyl dodecyl trithiocarbonate, 4-cyano-4-[(dodecylsulfanylthiocarbonyl)sulfanyl]pentanoic acid, cyanomethyldodecyl trithiocarbonate, and 2-(dodecylthiocarbonothioylthio)-2-methylpropionic acid. Examples of the dithioester compound include 1-ethoxycarbonyl-1-phenylmethyl benzodithioate, 2-phenyl-2-propylbenzodithioate, 4-cyano-4-(phenylthiocarbonylthio)pentanoic acid, and 2-cyano-2-propylbenzodithioate.

Of the foregoing RAFT agents, it is preferred from the aspect of availability to select 2-cyano-2-propyl dodecyl trithiocarbonate as the trithiocarbonate compound and 1-ethoxycarbonyl-1-phenylmethyl benzodithioate as the dithioester compound.

Further, in view of reactivity with the methacrylate monomer, 2-cyano-2-propyldodecyl trithiocarbonate which is an acidic group-free trithiocarbonate compound is optimum. The RAFT agent having a cyano group is highly reactive with the methacrylate monomer, as compared with analogous RAFT agents having an amino or amide group. Because of the lack of a conjugated system such as aromatic ring within the molecule, this compound does not detract from transmittance in the ArF immersion lithography and is thus suited in the ArF immersion lithography.

The amount of the RAFT agent used in step (1) is preferably at least 0.05 part by weight, more preferably at least 0.1 part by weight per 100 parts by weight of the monomers in total. As the upper limit, the amount is preferably up to 20 parts by weight, more preferably up to 10 parts by weight per 100 parts by weight of the monomers in total. The RAFT agent may be used alone or in admixture of two or more.

### <Polymerization Initiator>

Examples of the polymerization initiator include 2,2′-azobisisobutyronitrile (AIBN), 2,2′-azobis(2,4-dimethylvaleronitrile), dimethyl 2,2-azobis(2-methylpropionate), 1,1′-azobis(1-acetoxy-1-phenylethane), benzoyl peroxide, and lauroyl peroxide. The amount of the polymerization initiator used is preferably 0.01 to 25 mol % based on the total of monomers to be polymerized.

### <Polymerization Solvent>

In step (1), the living radical polymerization is preferably performed by solution polymerization for the purpose of uniformly feeding monomers to the reaction system. The solvent (S) used for polymerization reaction is preferably selected from compounds having the formula (S-1) and compounds having the formula (S-2).

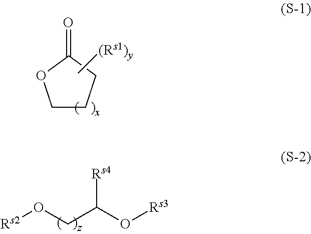

In formulae (S-1) and (S-2), R^{s1} is hydrogen, hydroxy, or an optionally substituted C₁-C₈ saturated hydrocarbyl group. R^{s2} to R^{s4} are each independently hydrogen, an optionally substituted C₁-C₈ saturated hydrocarbyl group or optionally substituted C₂-C₉ saturated hydrocarbylcarbonyl group. The subscript x is 1, 2 or 3, y is 0, 1 or 2, and z is 1, 2 or 3.

The optionally substituted C₁-Cₛ saturated hydrocarbyl group represented by R^{s1} to R^{s4} and saturated hydrocarbyl moiety in the C₂-C₉ saturated hydrocarbylcarbonyl group represented by R^{s2} to R^{s4} may be straight, branched or cyclic and examples thereof include alkyl groups such as methyl, ethyl, propyl, isopropyl, n-butyl, sec-butyl, tert-butyl, pentyl, hexyl, heptyl, octyl; cyclic saturated hydrocarbyl groups such as cyclopentyl and cyclohexyl; and substituted forms of the foregoing groups in which some hydrogen is substituted by hydroxy or the like.

Examples of the solvent having formula (S-1) are shown below, but not limited thereto.

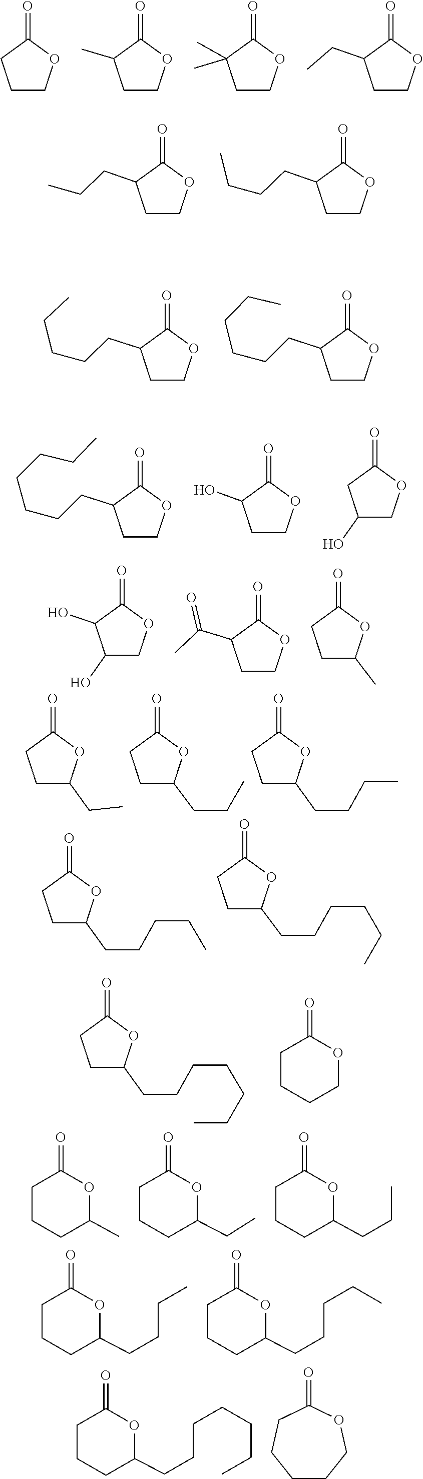

Examples of the solvent having formula (S-2) are shown below, but not limited thereto.

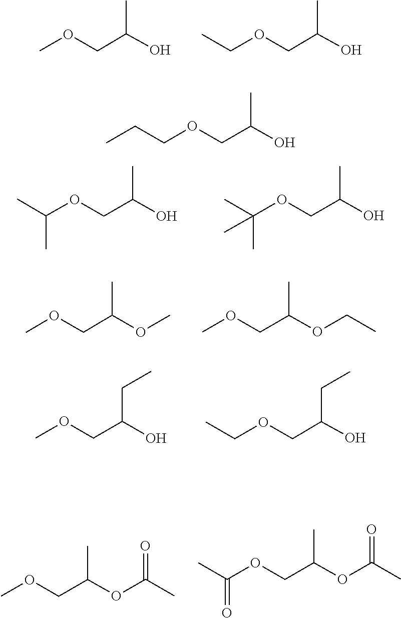

The amount of the solvent (S) used is preferably 1 to 100% by weight, more preferably 10 to 100% by weight, even more preferably 20 to 100% by weight based on the overall solvents used for polymerization.

Besides, examples of the organic solvent which can be used in polymerization include toluene, benzene, tetrahydrofuran (THF), diethyl ether, dioxane and methyl ethyl ketone (MEK), other than the above-mentioned examples of solvent (S). Any of these solvents may be used in admixture with solvent (S). If necessary, the step of bubbling nitrogen stream or vacuum pumping may be carried out prior to polymerization reaction to remove any dissolved oxygen in the solvent out of the reaction system.

In step (1), the monomer concentration of the monomer solution is preferably set at 30 to 60% by weight. The living radical polymerization under such conditions makes it possible that the monomers be fully consumed within such a reaction time as not to detract from the production efficiency and the contents of residual monomers after polymerization and after purification be held at low levels.

The living radical polymerization may be carried out by a method which is selected from methods (1) to (3):
method (1) of admitting the monomers, polymerization initiator and RAFT agent all at once to solvent (S) in a reactor, dissolving them, heating the reactor to initiate reaction;
method (2) of charging a reactor with part of solvent (S), preheating the reactor, and feeding a solution of the monomers, polymerization initiator and RAFT agent in solvent (S) to the reactor to initiate reaction; and
method (3) of charging a reactor with the RAFT agent and part of solvent (S), preheating the reactor, and feeding a solution of the monomers and polymerization initiator in solvent (S) to the reactor to initiate reaction.
   When solutions of monomers, initiator and RAFT agent used in methods (2) and (3) are prepared, it is acceptable to prepare separate solutions of components and independently feed them to the reactor. Since there is a possibility that polymerization reaction take place with radicals generated from the initiator in the standby time, to form ultra-high molecular weight polymers, it is preferred from the standpoint of quality control to prepare at least the monomer solution and the initiator solution independently and add them dropwise.

In the living radical polymerization, the reaction temperature is preferably 50 to 150° C., more preferably 60 to 100° C., and the reaction time is preferably 2 to 24 hours, and more preferably 2 to 12 hours from the aspect of production efficiency. The amounts of monomers A1, A2, A3 and A4 remaining in the reaction solution containing polymer P-1 at the end of living radical polymerization each are preferably less than 2.0% by weight. It is noted that the amounts of residual monomers can be quantitatively determined by high-performance liquid chromatography.

If necessary, the step of living radical polymerization may be followed by the purification step of adding the reaction solution to a poor solvent and allowing the polymer to reprecipitate. The poor solvent used herein may be selected depending on the type of the polymer. Typical examples of the poor solvent include, but are not limited to, hydrocarbons such as toluene, xylene, hexane, and heptane, ethers such as diethyl ether, tetrahydrofuran, and dibutyl ether, ketones such as acetone and 2-butanone, esters such as ethyl acetate and butyl acetate, alcohols such as methanol and ethanol, and water. These solvents may be used alone or in admixture.

### [Step (2)]

The subsequent step (2) is to add the radical generator and a thiol compound to the solution containing polymer P-1 resulting from step (1), and heating the solution to convert the end structure to hydrogen, yielding a polymer P. Through this step, the end structure of polymer P-1 is replaced by hydrogen. Although some of the radical generator used in step (1) may remain undecomposed in the solution at this point of time, it is recommended to supplement the radical generator at the same time as the addition of the thiol compound for the purpose of quickly and efficiently replacing the end structure by hydrogen.

The radical generator used in step (2) may be selected from the same ones as exemplified in step (1). The amount of the radical generator used is preferably 0.5 to 10 moles, more preferably 0.5 to 2 moles per mole of the RAFT agent used in step (1).

The thiol compound used in step (2) is preferably a compound having the formula (SH-1) or (SH-2).

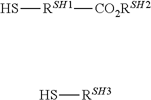

In formula (SH-1), R^{SH1} is a C_-C₃ hydrocarbylene group. Examples of the hydrocarbylene group include methanediyl, ethane-1,1-diyl, ethane-1,2-diyl, propane-1,1-diyl, propane-1,2-diyl, propane-1,3-diyl, and propane-2,2-diyl.

In formula (SH-1), R^{SH2} is a C_-C₈ aliphatic hydrocarbyl group which may contain a heteroatom, C₇-C₁₈ aralkyl group or C₆-C₁₈ aryl group. Suitable heteroatoms include oxygen, nitrogen, sulfur and halogen.

The aliphatic hydrocarbyl group R^{SH2} may be saturated or unsaturated and straight, branched or cyclic. Examples thereof include straight or branched aliphatic hydrocarbyl groups such as methyl, ethyl, n-propyl, isopropyl, n-butyl, isobutyl, sec-butyl, tert-butyl, n-pentyl, isopentyl, sec-pentyl, 3-pentyl, tert-pentyl, neopentyl, n-hexyl, 3-methylpentan-3-yl, 2,3-dimethylbutan-2-yl, n-heptyl, 2,3,4-trimethylpentan-3-yl, n-octyl, tetradecyl, hexadecyl, and octadecyl; and cyclic aliphatic hydrocarbyl groups such as cyclopentyl, 1-methylcyclopentyl, 1-ethylcyclopentyl, 1-vinylcyclopentyl, cyclohexyl, 1-methylcyclohexyl, 1-ethylcyclohexyl, 1-vinylcyclohexyl, norbornyl, 1-methylnorbornyl, cyclooctyl, cyclodecyl, cyclododecyl, 1-adamantyl, 2-adamantyl, 1-methyladamantyl, and 1-ethyladamantyl.

Examples of the C₇-C₁₈ aralkyl group R^{SH2} include benzyl, phenethyl, 4-methoxybenzyl, and 9-anthracenylmethyl. Examples of the C₆-C₁₈ aryl group R^{SH2} include phenyl, naphthyl, 4-methoxyphenyl, 2-anthracenyl and 9-anthracenyl.

In formula (SH-2), R^{SH3} is a C₂-C₂₀ saturated hydrocarbyl group which may contain a heteroatom, C₇-C₁₈ aralkyl group or C₆-C₁₈ aryl group.

The saturated hydrocarbyl group RSH³ may be straight, branched or cyclic. Examples thereof include ethyl, n-propyl, n-butyl, n-pentyl, isopropyl, 1-methylpropyl, 2-methylpropyl, 1-methylbutyl, 2-methylbutyl, 3-methylbutyl, cyclobutyl, cyclopentyl, n-hexyl, 3-methylpentyl, n-heptyl, n-octyl, 1-ethylhexyl, n-nonyl, n-decyl, n-undecyl, n-dodecyl, 2,2,4,6,6-pentamethylheptan-4-yl, n-tetradecyl, n-hexadecyl, n-octadecyl, n-eicosanyl, 2,3,3,4,4,5-hexamethylhexan-2-yl.

Examples of the C₇-C₁₈ aralkyl group R^{SH3} include benzyl, phenethyl, 4-methoxybenzyl, and 9-anthracenylmethyl. Examples of the C₆-C₁₈ aryl group R^{SH3} include phenyl, naphthyl, 4-methoxyphenyl, 2-anthracenyl and 9-anthracenyl.

In the co-presence of the radical initiator, the thiol compound plays the role of acting on the end of polymer P-1 resulting from step (1) to quickly convert the end to hydrogen. This mechanism is described in JP-A 2006-002096. Then, the re-generated RAFT agent, the active species (active species I) originating from the initiator, and the thio radicals (active species S) after release of hydrogen radicals are formed in the reaction system. Shown below is an exemplary reaction system wherein the end of polymer P-1 originates from trithiocarbonate, the radical initiator is dimethyl 2,2′-azobisisobutyrate, and the thiol is a compound having formula (SH-2).

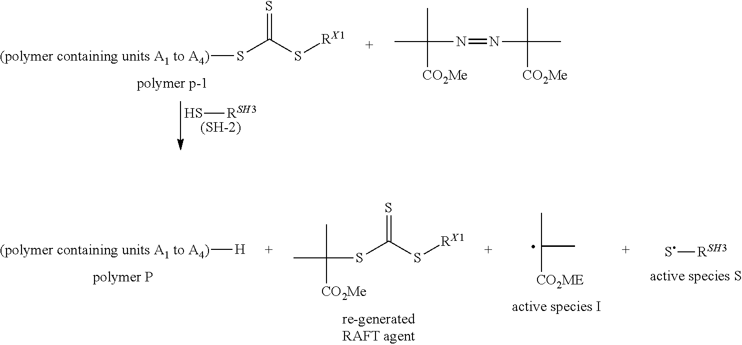

At the end of step (1), unreacted or residual monomers including monomers A1 to A4 remain in the solution containing polymer P-1, though in low concentrations. Side reactions take place along with the progress of step (2), that is, radical polymerization reaction steadily continues among the remaining monomers under the co-action of active species I, active species S, and re-generated RAFT agent as shown above, so that the amounts of residual monomers are further reduced.

When living radical polymerization is conducted to a full extent according to the method of step (1), the concentrations of residual monomers are quite low as compared with the concentrations of active species I originating from the initiator supplemented in step (2), active species S resulting from the thiol after conversion of the end of polymer P-1 to hydrogen, and the re-generated RAFT agent. For this reason, polymerization reaction among residual monomers taking place in step (2) does not form polymers having a high molecular weight, but forms oligomers mainly.

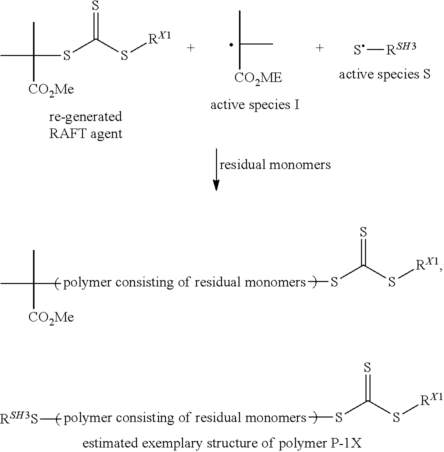

Since the polymer P-1X resulting from side reaction in step (2) has a low molecular weight, it is easily removed in the subsequent step (3). Due to the reaction mechanism involved, during polymerization of residual monomers, active species S originating from the thiol is incorporated at the end of polymer. Then, the substituent group on the thiol compound (SH-1) or (SH-2) is preferably selected such that the polymer is highly soluble in organic solvents and the polymer P-1X is readily removed in the polymer purifying step.

Taking the foregoing steps makes it possible to obtain a polymer having low contents of residual monomers. A resist composition comprising the polymer as a base polymer is minimized in defect-forming risk.

Preferred examples of the compound having formula (SH-1) are shown below.

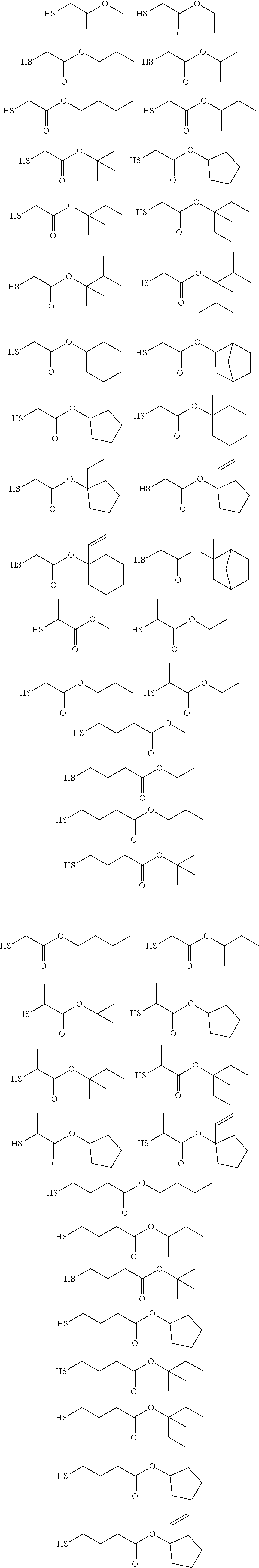

Preferred examples of the compound having formula (SH-2) are shown below.

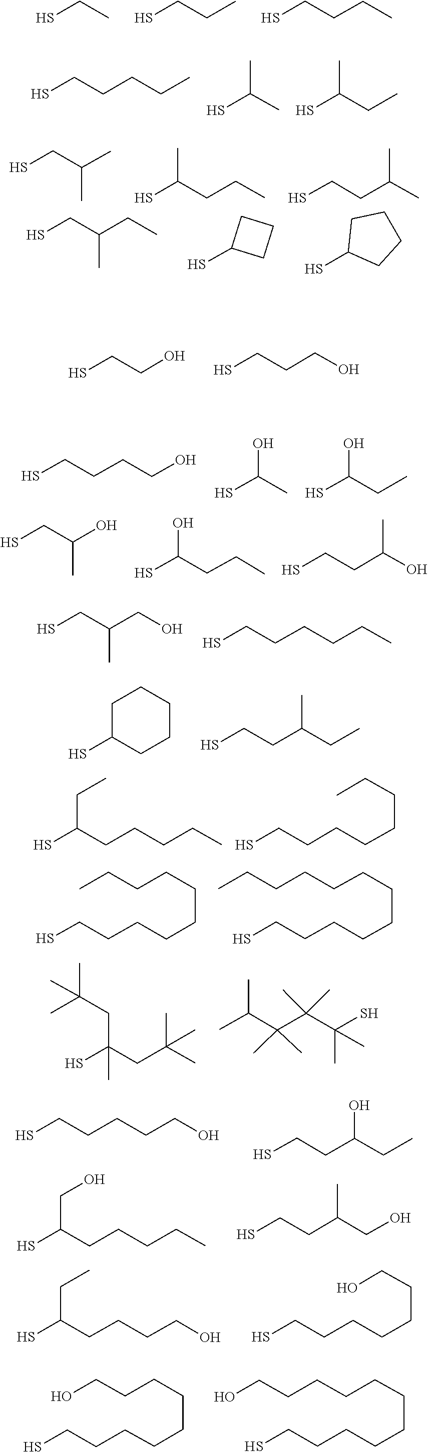

The amount of the thiol compound used in step (2) is preferably 1 to 20 moles, more preferably 1 to 4 moles per mole of the RAFT agent used in step (1). The radical initiator and thiol compound added in step (2) may be separately added to the solution containing polymer P-1. Alternatively, they may be mixed and concurrently added to the solution containing polymer P-1. From the aspect of operational efficiency, it is preferred to add a solution of the radical initiator and thiol compound in the solvent (S) to the polymer P-1 solution. More preferably, to the reaction solution resulting from living radical polymerization in step (1) in the solvent (S), the mixed solution of the initiator and thiol compound is added subsequent to step (1).

The reaction temperature in step (2) is preferably 50 to 150° C., more preferably 60 to 100° C. The reaction time in step (2) is preferably 2 to 24 hours, more preferably 2 to 5 hours from the aspect of production efficiency. Also, the solution containing polymer P in step (2) is preferably such that the amounts of monomers (A), (B), and (C) remaining therein each may be less than 0.05% by weight.

In step (2), the conversion rate (referred to as “end conversion rate,” hereinafter) from the end structure originating from the RAFT agent to hydrogen is preferably at least 98%, most preferably 100%. It is noted that the end conversion rate (%) is computed from the data of ¹H-NMR spectroscopy and represented by the following formula (i):

$$end conversion rate = 100 - [ \{ ( A 1 / R 1 ) / ( A 2 / R 2 ) \} \times 100 ] \qquad ( i )$$

wherein A1 is the integrated value of peaks assigned to the end group after contact of polymer P-1 with thiol-containing compound, A2 is the integrated value of peaks assigned to the end group before contact of polymer P-1 with thiol-containing compound, R1 is the integrated value of peaks assigned to the overall polymer excluding the end group after contact of polymer P-1 with thiol-containing compound, and R2 is the integrated value of peaks assigned to the overall polymer excluding the end group before contact of polymer P-1 with thiol-containing compound.

### [Step (3)]

The subsequent step (3) is a purification step of mixing the polymer P-containing solution resulting from step (2) with a poor solvent and allowing polymer P to precipitate as solids.

The method of obtaining the polymer as solids may be selected from the following methods:
method (I) of obtaining solids (or powder) directly from the reaction solution at the end of step (2), that is, by directly adding the polymer P-containing solution to a poor solvent,
method (II) of obtaining solids (or powder) by dissolving the polymer in another solvent while removing the solvent (S) used in steps (1) and (2) by vacuum distillation and adding the solution to a poor solvent,
method (III) of obtaining solids (or powder) by adding another solvent to the polymer solution, then adding water thereto, allowing the mixture to separate into the polymer solution as the upper layer and the mixture of solvent (S) and water as the lower layer, removing the lower layer, adjusting the concentration of the polymer solution, and adding it to a poor solvent, and
method (IV) of obtaining solids (or powder) by adding the polymer P-containing solution to water, allowing solids to precipitate, recovering the powder by filtration, dissolving the powder in another solvent, and adding the solution to a poor solvent.

Among these methods, method (I) or (III) is preferred from the aspects of operating time and precision.

The poor solvent may be selected depending on the type of the polymer. Typical examples of the poor solvent include, but are not limited to, hydrocarbons such as toluene, xylene, hexane, and heptane, ethers such as diethyl ether, tetrahydrofuran, diisopropyl ether, and dibutyl ether, ketones such as acetone and 2-butanone, esters such as ethyl acetate and butyl acetate, alcohols such as methanol and ethanol, and water. These solvents may be used alone or in admixture.

The purification method of obtaining a powder by adding the polymer solution to a poor solvent is employed for the purpose of removing low-molecular-weight polymer fractions formed in steps (1) and (2) and unreacted monomers. If necessary, similar purification may be repeated by dissolving the resulting powder to form a solution again.

In the polymer thus obtained from the inventive method, the amounts of monomer A1, A2, A3 and A4 remaining therein each are preferably up to 0.05% by weight.

The polymer obtained from the inventive method preferably has a weight average molecular weight (Mw) of 1,000 to 30,000, more preferably 3,000 to 20,000. It is noted that Mw is as measured by gel permeation chromatography (GPC) versus polystyrene standards. Also, the polymer preferably has a dispersity (Mw/Mn) of 1.0 to 1.5, more preferably 1.0 to 1.35.

### [Preparation of Polymer Solution]

Preferably the polymer P obtained from the inventive method is dissolved in a solvent to form a polymer solution (referred to as polymer solution PS, hereinafter), which is handled as the final product. The solvent contains at least propylene glycol monomethyl ether acetate (PGMEA). The solvent may contain a solvent other than PGMEA. Examples of the other solvent include those described in JP-A 2008-111103, paragraphs [0144]-[0145], specifically ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as γ-butyrolactone (GBL); alcohols such as diacetone alcohol (DAA); and high-boiling alcohols such as diethylene glycol, propylene glycol, glycerin, 1,4-butanediol, and 1,3-butanediol. The other solvent may be used alone or in admixture. When the solvent contains the other solvent, the content of PGMEA is preferably 25 to 80% by weight, more preferably 30 to 70% by weight of the overall solvents.

After the polymer P is dissolved in the solvent, it is passed through a filter to yield a polymer solution PS. The filtration step is effective for quality stabilization because foreign particles or gel which cause defects can be removed.

Examples of the material of which the filter used for filtration is made include fluorocarbon, cellulose, nylon, polyester, and hydrocarbon based materials. In the step of filtering resist compositions, filters made of materials based on fluorocarbon, commonly known as Teflon®, materials based on hydrocarbons such as polyethylene and polypropylene, or nylon are preferred. The pore size of the filter may be selected as appropriate so as to meet the desired degree of cleanness, and is preferably up to 100 nm, more preferably up to 20 nm. The filter may be used alone or in a combination of two or more.

The filtration step may be performed by passing the solution only once through the filter, but preferably by circulating the solution so as to pass through the filter plural times. The filtration step may be performed in any order and any times in the method of preparing the polymer. It is preferred to filter the reaction solution after polymerization reaction or polymer solution PS or both.

The concentration of polymer P in the polymer solution PS is preferably 0.01 to 30% by weight, more preferably 0.1 to 20% by weight.

### [Preparation of Chemically Amplified Resist Composition]

A further embodiment of the invention is a method for preparing a chemically amplified resist composition. The resist composition contains several components, which are described below.

### <Base Polymer>

The chemically amplified resist composition contains a base polymer containing the polymer obtained from the aforementioned method. The polymer may be used alone or in a combination of two or more polymers which are different in compositional ratio, Mw and dispersity. In the base polymer, the content of the polymer is preferably at least 40% by weight, more preferably at least 80% by weight, most preferably 100% by weight.

### <Photoacid Generator>

The chemically amplified resist composition also contains a photoacid generator. The photoacid generator is not particularly limited as long as it is capable of generating an acid upon exposure to high-energy radiation. Typical of the photoacid generator are compounds having the formulae (B1), (B2), and (B3).

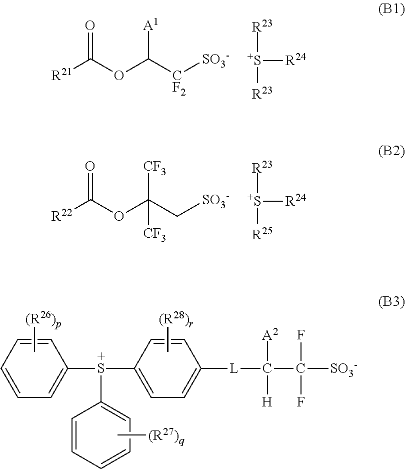

In formula (B1), A¹ is hydrogen or trifluoromethyl.

In formulae (B1) and (B2), R²¹ and R²² are each independently a C₁-C₃₅ hydrocarbyl group which may contain a heteroatom, a C₂-C₃₅ monovalent nitrogen-containing heterocyclic group or a group having the formula (b).

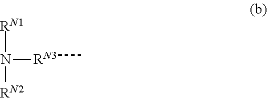

In formula (b), R^{N1} and R^{N2} are each independently hydrogen or a C₁-C₂₀ hydrocarbyl group which may contain a heteroatom. R^{N1} and R^{N2} may bond together to form a ring with the nitrogen atom to which they are attached. R^{N3} is a C₁-C₂₀ hydrocarbylene group which may contain a heteroatom.

The C₁-C₃₅ hydrocarbyl group represented by R²¹ and R²² may be saturated or unsaturated and straight, branched or cyclic. Examples thereof include C₁-C₃₅ alkyl groups such as methyl, ethyl, propyl, isopropyl, n-butyl, sec-butyl, tert-butyl, pentyl, hexyl, heptyl and octyl; C₃-C₃₅ cyclic saturated hydrocarbyl groups such as cyclopentyl, cyclohexyl, cycloheptyl, cyclopropylmethyl, 4-methylcyclohexyl, cyclohexylmethyl, norbornyl, 1-adamantyl and 1-adamantylmethyl; steroid structure-containing groups; C₂-C₃₅ alkenyl groups such as vinyl and isopropenyl; C₆-C₃₅ aryl groups such as phenyl, 1-naphthyl, 2-naphthyl, anthryl, alkylphenyl groups (e.g., 2-methylphenyl, 3-methylphenyl, 4-methylphenyl, 4-ethylphenyl, 4-tert-butylphenyl, 4-n-butylphenyl), dialkylphenyl groups (e.g., 2,4-dimethylphenyl), alkylnaphthyl groups (e.g., methylnaphthyl and ethylnaphthyl), and dialkylnaphthyl groups (e.g., dimethylnaphthyl and diethylnaphthyl); and C₇-C₃₅ aralkyl groups such as benzyl, 1-phenylethyl and 2-phenylethyl, and combinations thereof.

The hydrocarbyl group may contain oxygen. Examples of the oxygen-containing hydrocarbyl group include oxo-substituted saturated hydrocarbyl groups such as 2-oxocyclopentyl, 2-oxocyclohexyl, 4-oxocyclohexyl, 2-oxopropyl, 2-oxoethyl, 2-cyclopentyl-2-oxoethyl, 2-cyclohexyl-2-oxoethyl, 2-(4-methylcyclohexyl)-2-oxoethyl, 4-oxa-tricyclo[4.2.1.0^{3,7}]nonan-5-on-9-yl, and 4-oxo-1-adamantyl; hydroxyphenyl groups such as 4-hydroxyphenyl; alkoxyphenyl groups such as 4-methoxyphenyl, 3-methoxyphenyl, 2-methoxyphenyl, 4-ethoxyphenyl, 4-tert-butoxyphenyl and 3-tert-butoxyphenyl; alkoxynaphthyl groups such as methoxynaphthyl and ethoxynaphthyl; dialkoxynaphthyl groups such as dimethoxynaphthyl and diethoxynaphthyl; and aryloxoalkyl groups, e.g., 2-aryl-2-oxoethyl groups such as 2-phenyl-2-oxoethyl, 2-(1-naphthyl)-2-oxoethyl, and 2-(2-naphthyl)-2-oxoethyl.

Examples of the monovalent nitrogen-containing heterocyclic group represented by R²¹ and R²² include monovalent groups derived from aziridine, pyrrolidine, piperidine, morpholine, pyrrole, pyridine, azetidine, oxazole, isoxazole, thiazole, isothiazole, imidazole, pyrazole, pyridazine, pyrimidine, pyrazine, pyrroline, 2-imidazoline, imidazolidine, 3-pyrazoline, pyrazolidine, piperazine, triazine, oxadiazine, dithiazine, indole, isoindole, quinoline, isoquinoline, cinnoline, phthalazine, quinazoline, quinoxaline, 1,8-naphthylidine, purine, pteridine, indolizine, carbazole, acridine, phenazine, phenanthridine, 1,10-phenanthroline, phenoxazine, indoline, isoindoline, quinuclidine, benzo[e]indole, and benzo[cd]indole.

R²¹ and R²² are more preferably selected from tert-butyl, cyclohexyl, 1-adamantyl, 1-adamantylmethyl, 4-oxa-tricyclo[4.2.1.0^{3,7}]nonan-5-on-9-yl, 4-oxo-1-adamantyl, and steroid structure-containing alkyl groups.

The C₁-C₂₀ hydrocarbyl group represented by R^{N1} and R^{N2} may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, pentyl, hexyl, heptyl and octyl; C₃-C₂₀ cyclic saturated hydrocarbyl groups such as cyclopentyl, cyclohexyl, cycloheptyl, cyclopropylmethyl, 4-methylcyclohexyl, cyclohexylmethyl, norbornyl, and adamantyl; steroid structure-containing groups; C₂-C₂₀ alkenyl groups such as vinyl, 1-propenyl, 2-propenyl, butenyl and hexenyl; C₃-C₂₀ cyclic unsaturated hydrocarbyl groups such as cyclohexenyl; C₆-C₂₀ aryl groups such as phenyl, naphthyl, and thienyl; C₇-C₂₀ aralkyl groups such as benzyl, 1-phenylethyl and 2-phenylethyl, and combinations thereof. In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—), or haloalkyl moiety.

When R^{N1} and R^{N2} bond together to form a ring with the nitrogen atom to which they are attached, examples of the ring include aziridine, pyrrolidine, piperidine, morpholine, pyrrole, pyridine, azetidine, oxazole, isoxazole, thiazole, isothiazole, imidazole, pyrazole, pyridazine, pyrimidine, pyrazine, pyrroline, 2-imidazoline, imidazolidine, 3-pyrazoline, pyrazolidine, piperazine, triazine, oxadiazine, dithiazine, indole, isoindole, quinoline, isoquinoline, cinnoline, phthalazine, quinazoline, quinoxaline, 1,8-naphthylidine, purine, pteridine, indolizine, carbazole, acridine, phenazine, phenanthridine, 1,10-phenanthroline, phenoxazine, indoline, isoindoline, quinuclidine, benzo[e]indole, and benzo[cd]indole. In the ring, some or all hydrogen may be substituted by a C₁-C₂₀ hydrocarbyl group or a group containing a heteroatom such as oxygen, sulfur, nitrogen or halogen, and some —CH₂— may be replaced by a group containing a heteroatom such as oxygen, sulfur or nitrogen, so that the ring may contain a hydroxy group, fluorine, chlorine, bromine, iodine, cyano group, carbonyl group, ether bond, ester bond, sulfonate ester bond, carbonate bond, lactone ring, sultone ring, carboxylic anhydride (—C(═O)—O—C(═O)—), or haloalkyl group.

In formula (b), examples of the hydrocarbylene group R^{N3} include C₁-C₂₀ straight alkanediyl groups such as methanediyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-dyl, and heptadecane-1,17-diyl; C₂-C₂₀ branched alkanediyl groups corresponding to the foregoing straight alkanediyl groups in which some hydrogen is substituted by an alkyl moiety such as methyl, ethyl, propyl, isopropyl, butyl, sec-butyl, or tert-butyl; C₃-C₂₀ cyclic saturated hydrocarbyl groups such as cyclopentanediyl, cyclohexanediyl, norbornanediyl and adamantanediyl; arylene groups such as phenylene and naphthylene, and combinations thereof. In the hydrocarbylene group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—), or haloalkyl moiety.

Exemplary structures of the anion of the photoacid generator having formula (B1) are shown below, but not limited thereto. Herein A¹ is as defined above.

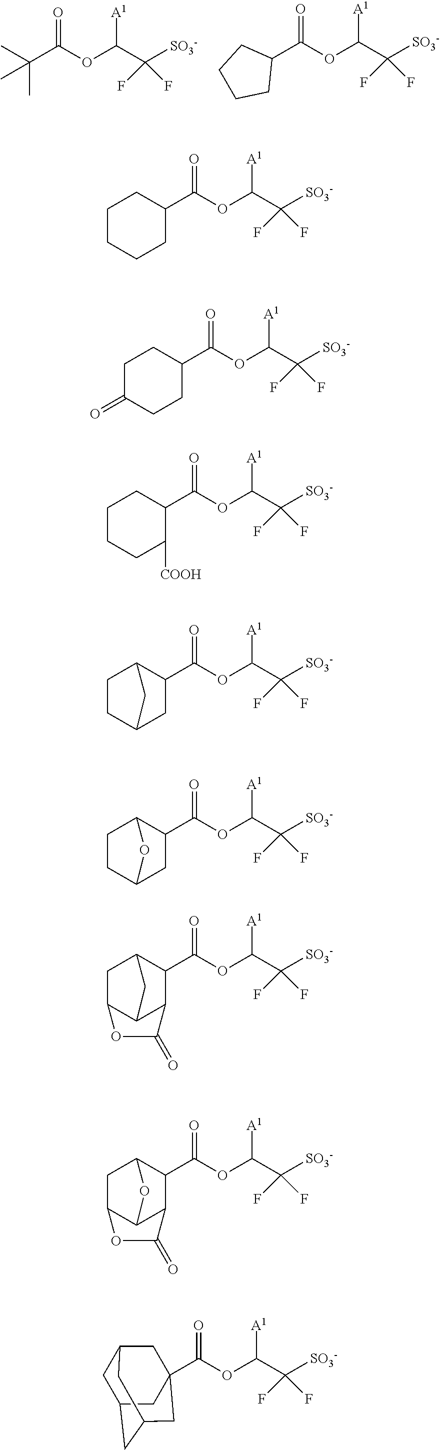

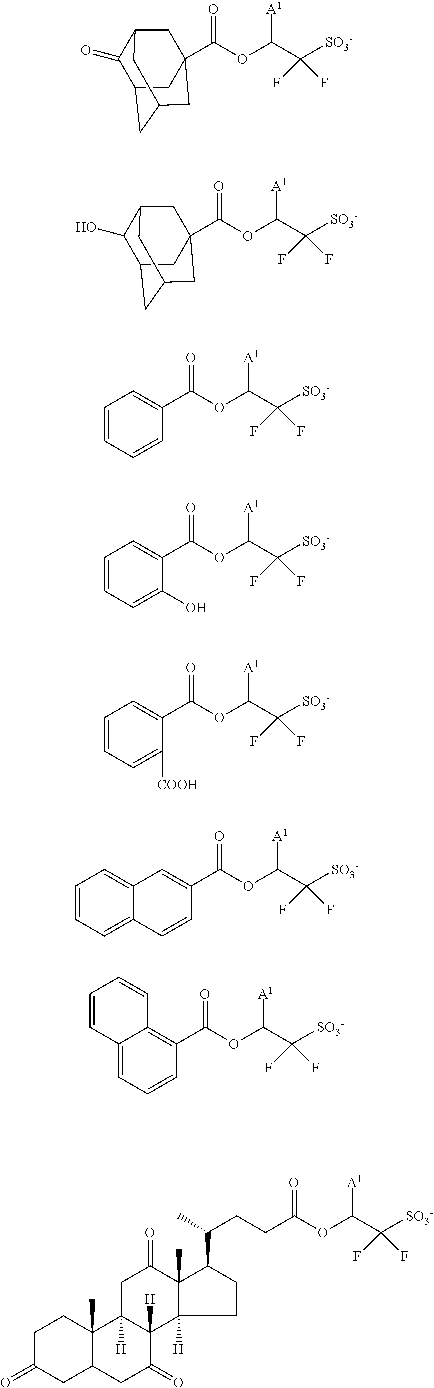

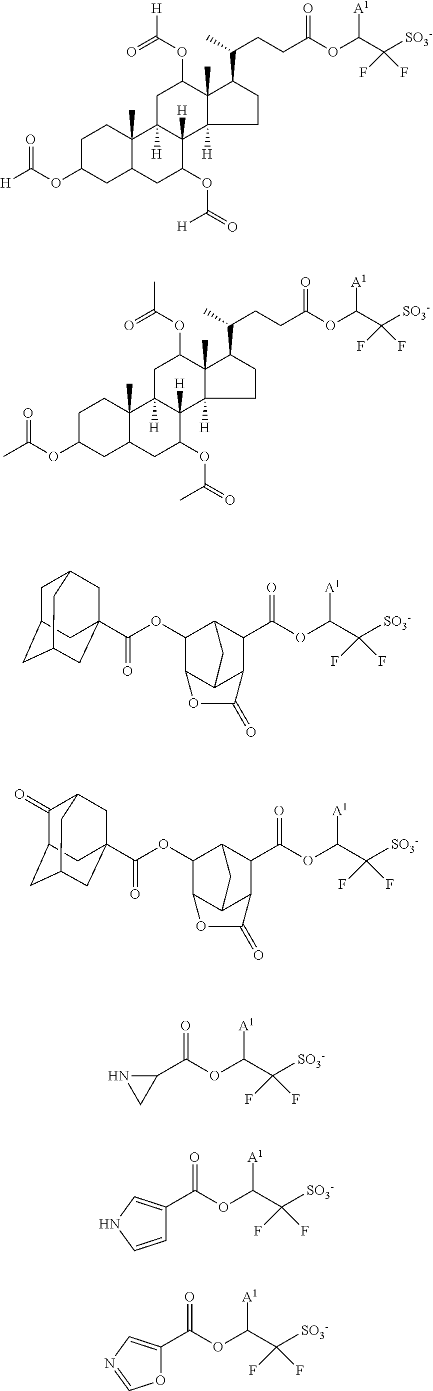

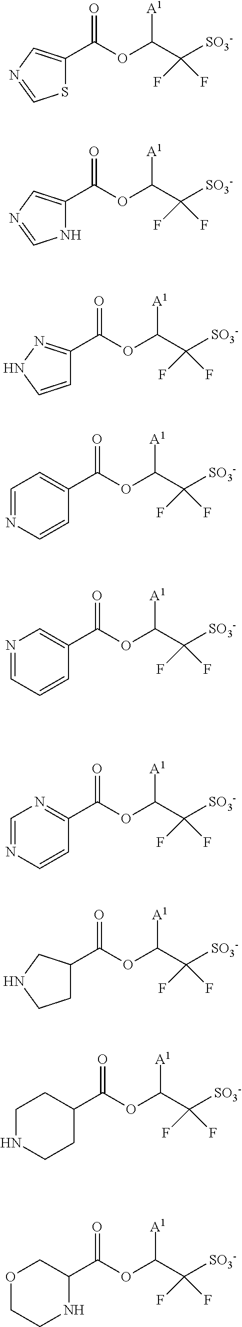

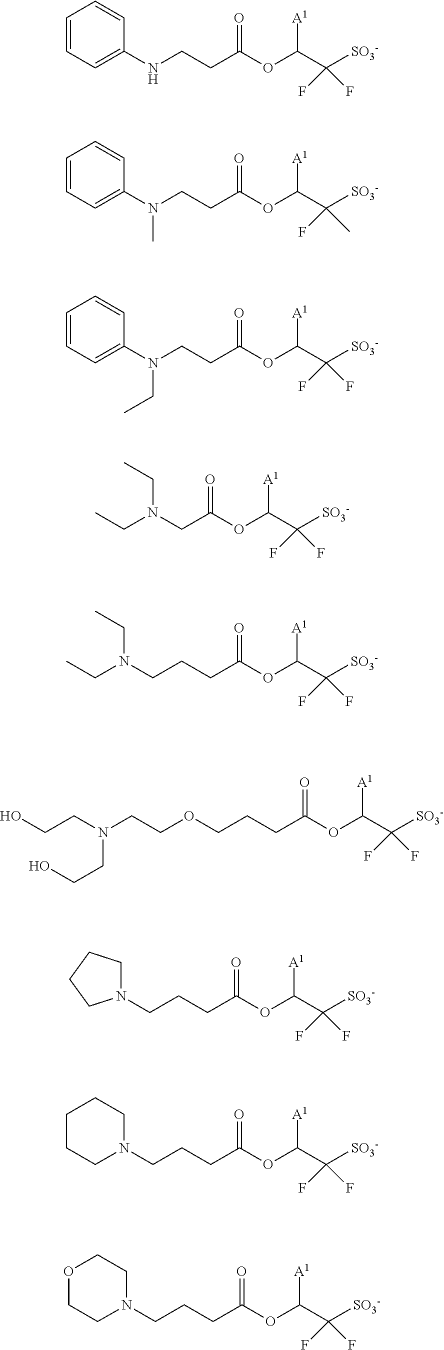

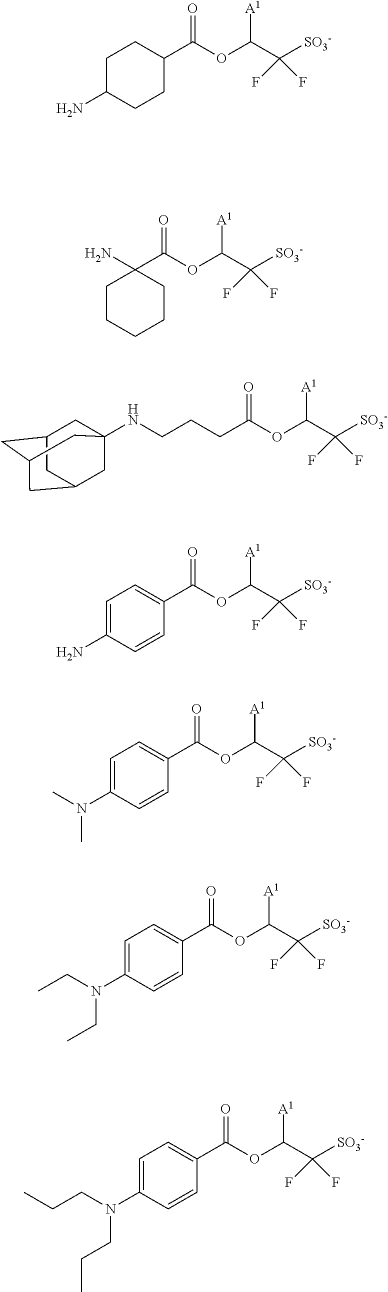

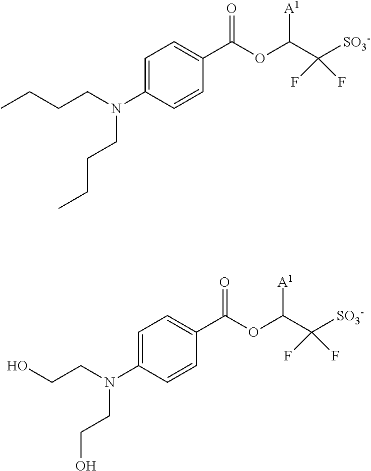

Exemplary structures of the anion of the photoacid generator having formula (B2) are shown below, but not limited thereto.

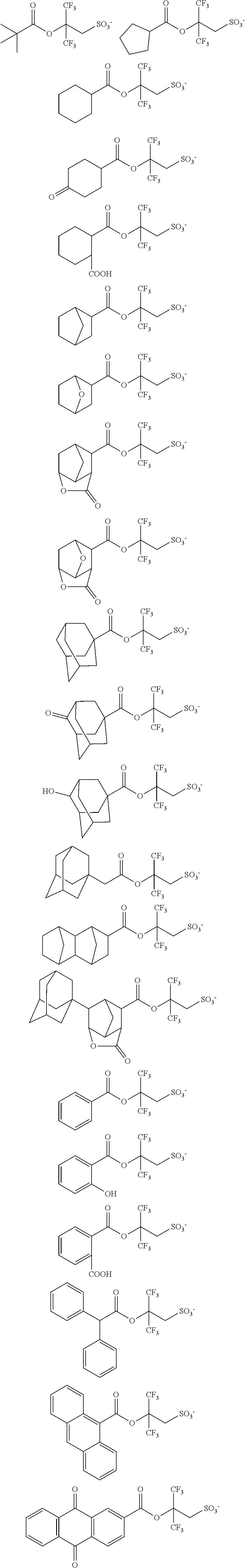

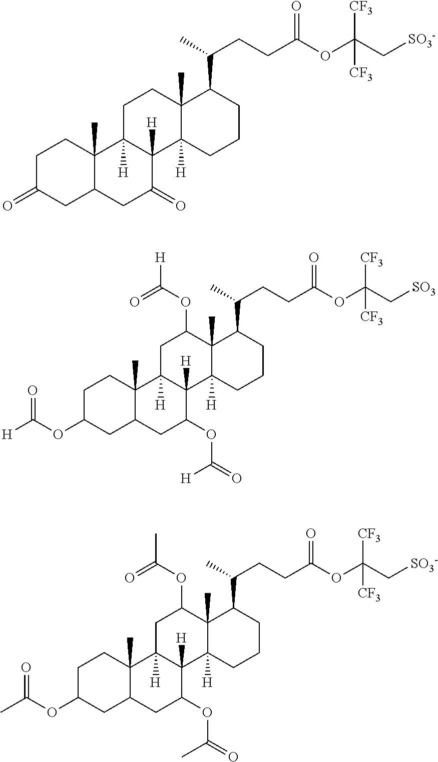

In formula (B1) and (B2), R²³, R²⁴ and R²⁵ are each independently halogen or a C₁-C₃₀ hydrocarbyl group which may contain a heteroatom.

Exemplary of the halogen represented by R²³ to R²⁵ are fluorine, chlorine, bromine and iodine.

The hydrocarbyl group represented by R²³ to R²⁵ may be saturated or unsaturated and straight, branched or cyclic. Examples thereof include C₁-C₃₀ alkyl groups such as methyl, ethyl, propyl, isopropyl, n-butyl, sec-butyl, tert-butyl, pentyl, hexyl, heptyl and octyl; C₃-C₃₀ cyclic saturated hydrocarbyl groups such as cyclopentyl, cyclohexyl, cycloheptyl, cyclopropylmethyl, 4-methylcyclohexyl, cyclohexylmethyl, norbornyl and adamantyl; C₂-C₃₀ alkenyl groups such as vinyl, 1-propenyl, 2-propenyl, butenyl and hexenyl; C₃-C₃₀ cyclic unsaturated hydrocarbyl groups such as cyclohexenyl; C₆-C₃₀ aryl groups such as phenyl, alkylphenyl groups (e.g., 2-methylphenyl, 3-methylphenyl, 4-methylphenyl, 4-ethylphenyl, 4-tert-butylphenyl, 4-n-butylphenyl, and 2,4-dimethylphenyl), naphthyl, methylnaphthyl and ethylnaphthyl; and C₇-C₃₀ aralkyl groups such as benzyl, 1-phenylethyl and 2-phenylethyl, and combinations thereof. Of these, aryl groups are preferred.

In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy moiety, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—), or haloalkyl moiety. Examples of the heteroatom-containing hydrocarbyl group include heteroaryl groups such as thienyl; oxoalkyl groups such as 2-oxocyclopentyl, 2-oxocyclohexyl, 2-oxopropyl, 2-oxoethyl, 2-cyclopentyl-2-oxoethyl, 2-cyclohexyl-2-oxoethyl, 2-(4-methylcyclohexyl)-2-oxoethyl; hydroxyphenyl groups such as 4-hydroxyphenyl; alkoxyphenyl groups such as 4-methoxyphenyl, 3-methoxyphenyl, 2-methoxyphenyl, 4-ethoxyphenyl, 4-tert-butoxyphenyl, 3-tert-butoxyphenyl; alkoxynaphthyl groups such as methoxynaphthyl and ethoxynaphthyl; dialkoxynaphthyl groups such as dimethoxynaphthyl and diethoxynaphthyl; and aryloxoalkyl groups, typically 2-aryl-2-oxoethyl groups such as 2-phenyl-2-oxoethyl, 2-(1-naphthyl)-2-oxoethyl and 2-(2-naphthyl)-2-oxoethyl. Examples of the heteroatom-containing hydrocarbyl group include aryl groups having a polymerizable substituent group such as acryloyloxy and methacryloyloxy, specifically 4-acryloyloxyphenyl, 4-methacryloyloxyphenyl, 4-acryloyloxy-3,5-dimethylphenyl, 4-methacryloyloxy-3,5-dimethylphenyl, 4-vinyloxyphenyl, and 4-vinylphenyl.

Any two of R²³, R²⁴ and R²⁵ may bond together to form a ring with the sulfur atom to which they are attached. Exemplary structures of the ring are shown below.

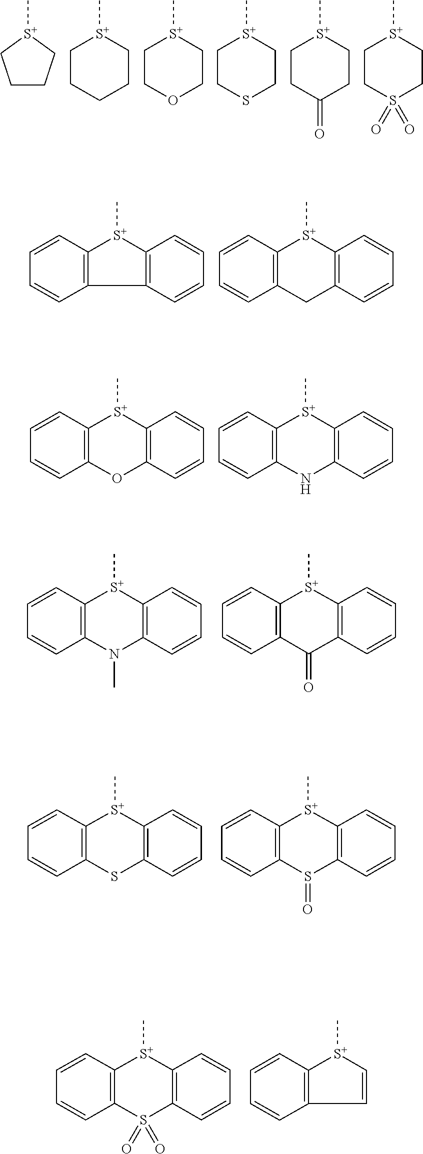

The broken line designates a point of attachment to R²⁵.

Exemplary structures of the cation of the photoacid generators having formulae (B1) and (B2) are shown below, but not limited thereto.

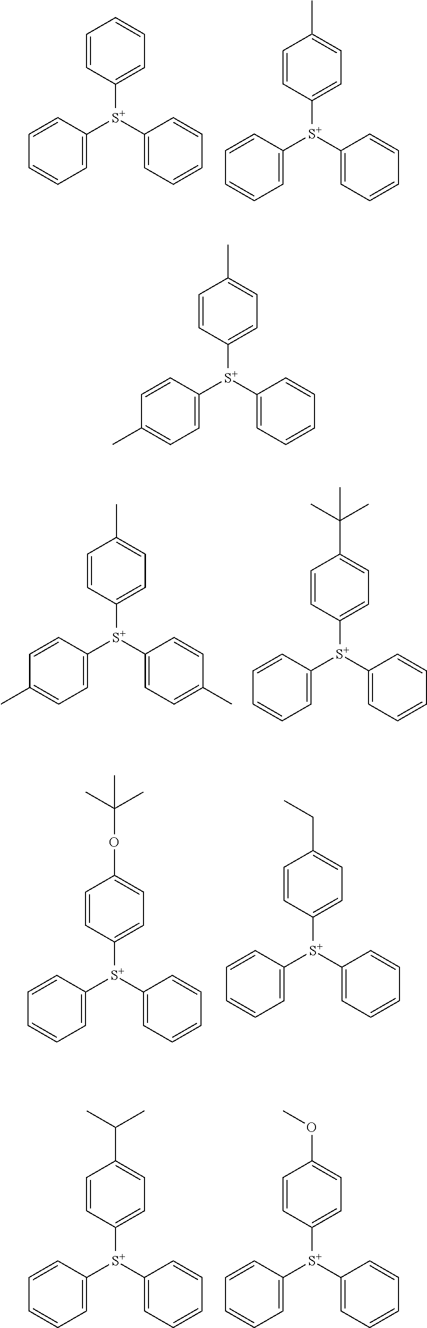

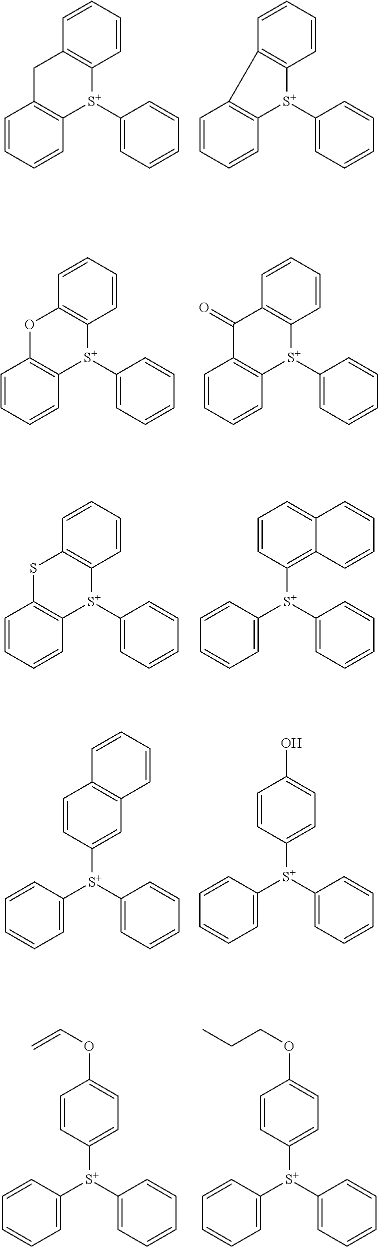

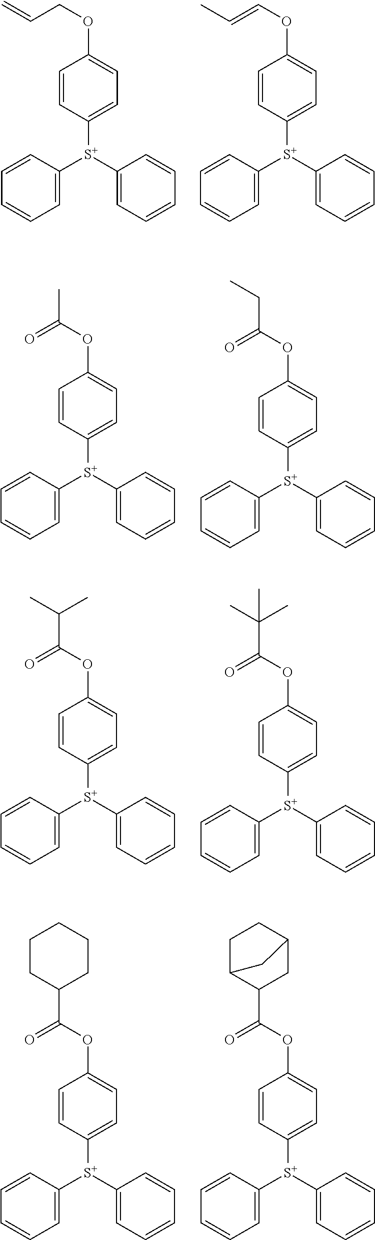

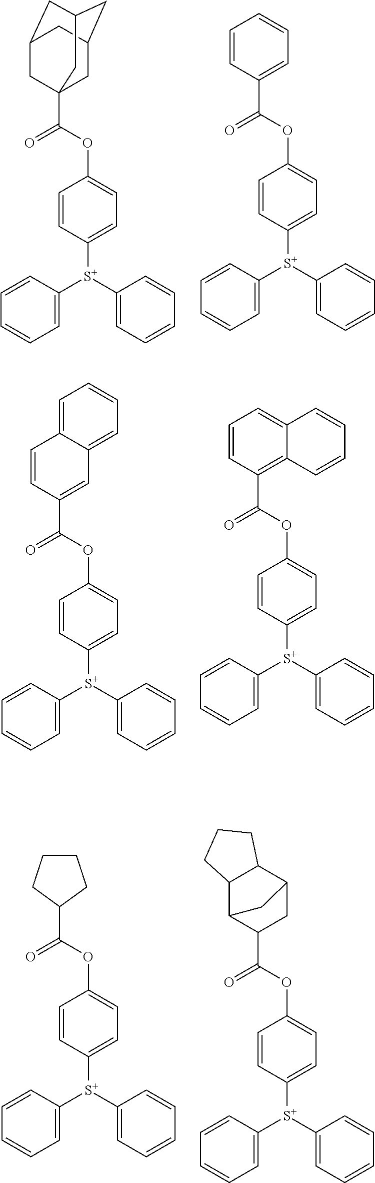

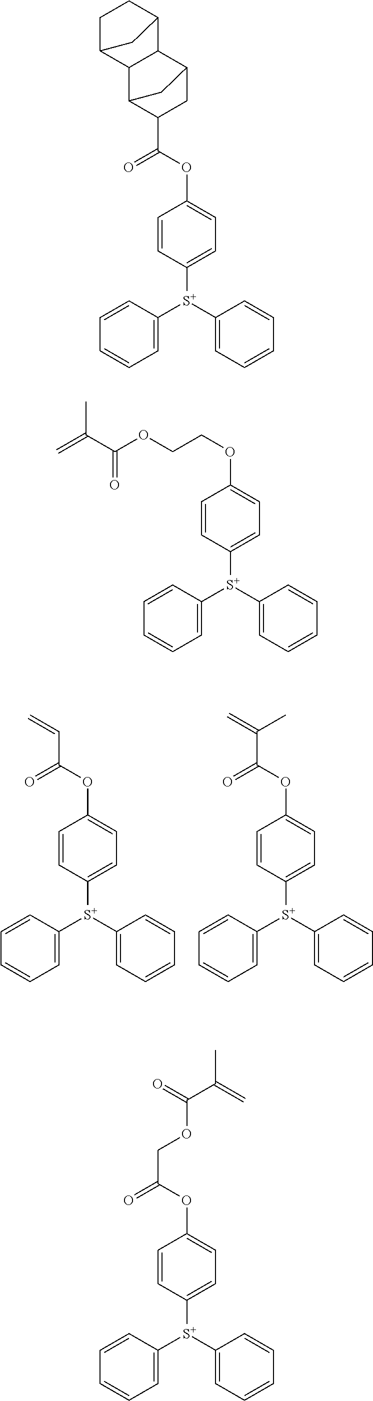

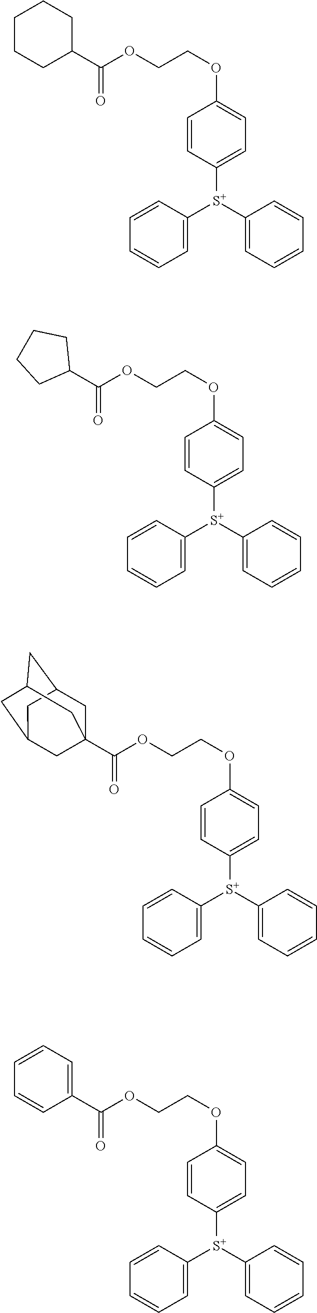

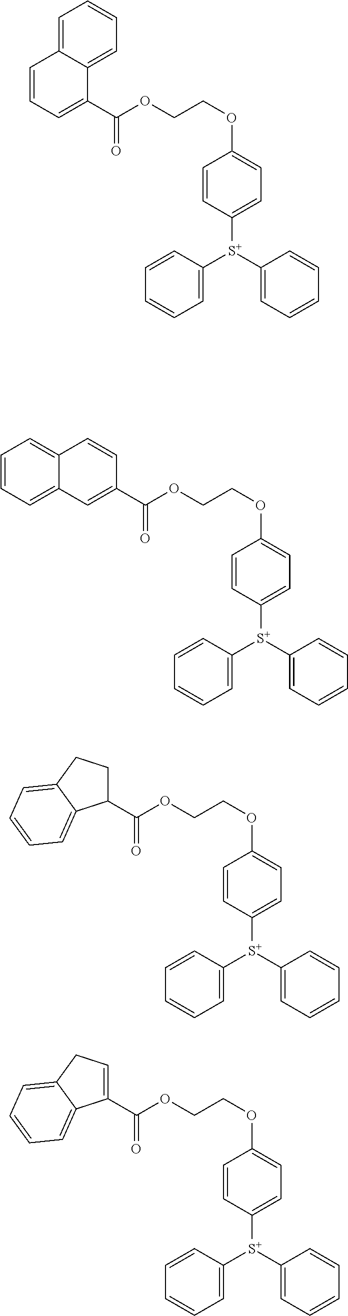

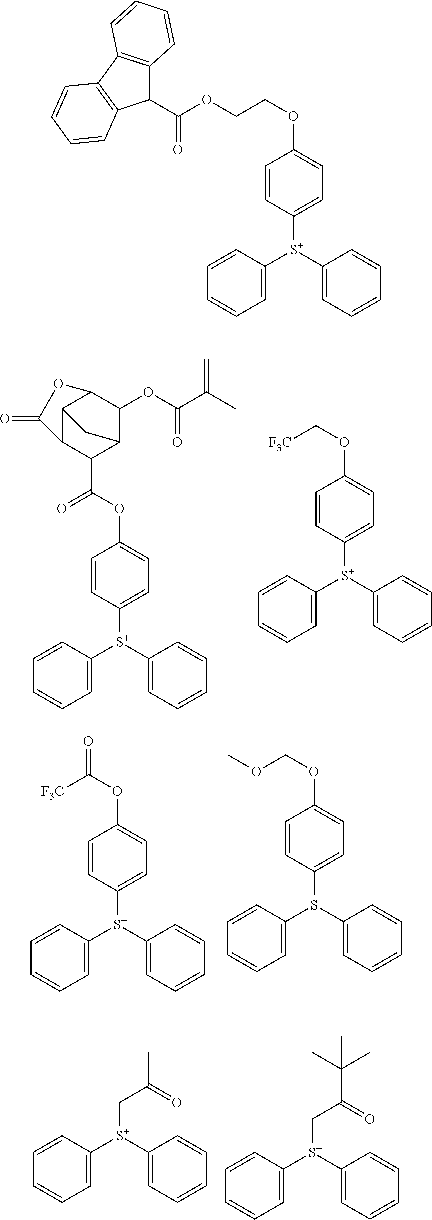

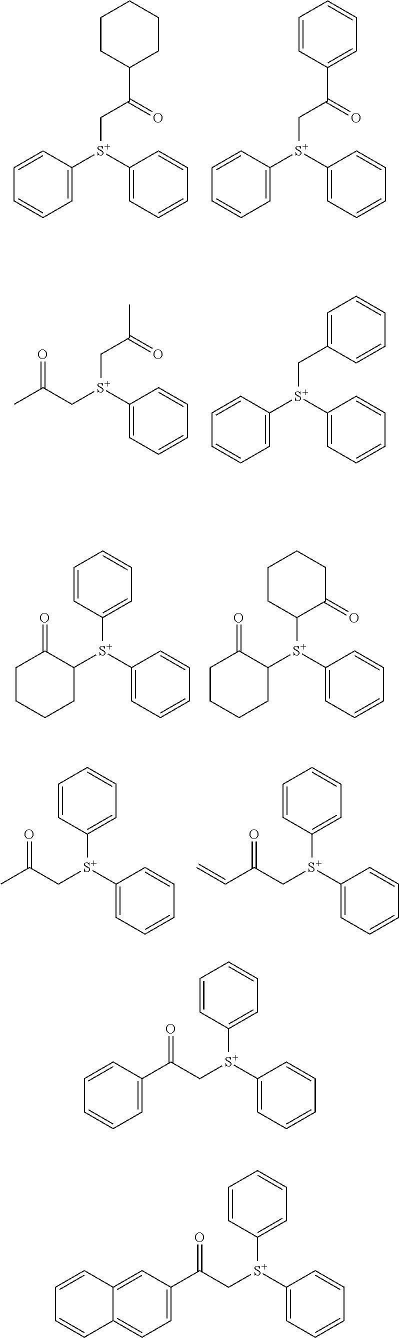

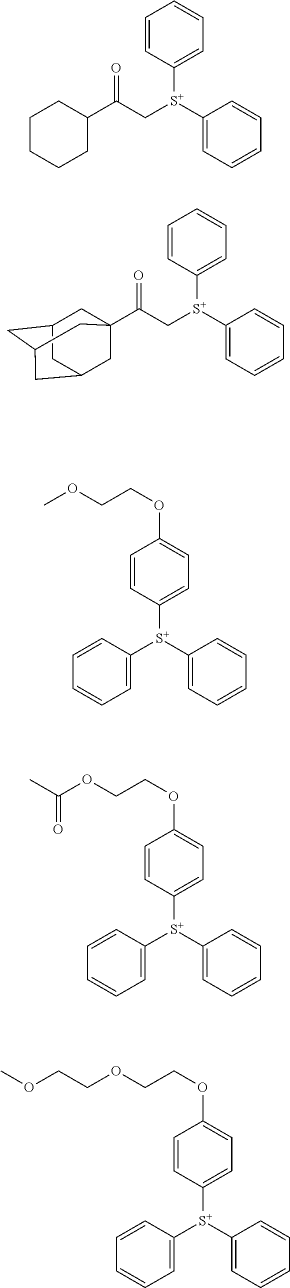

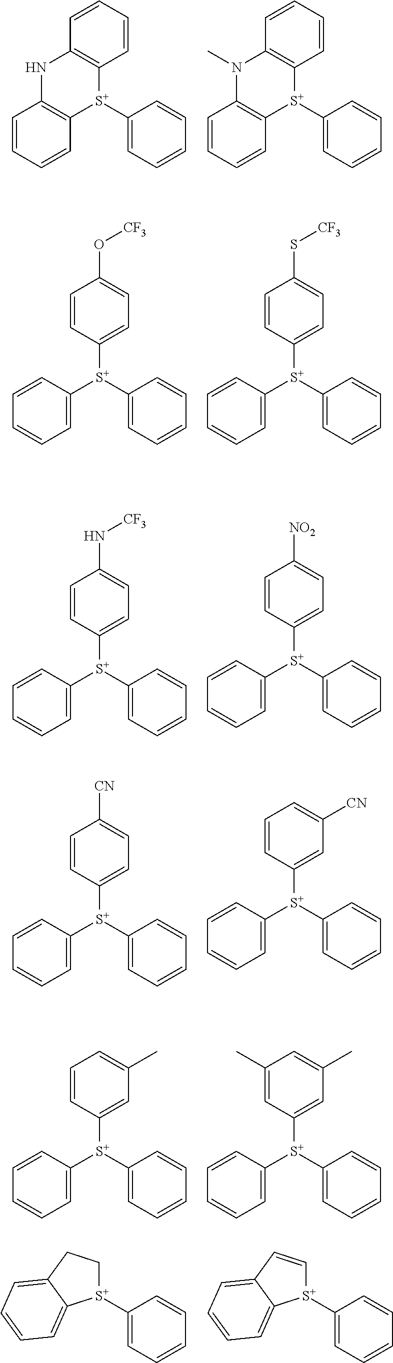

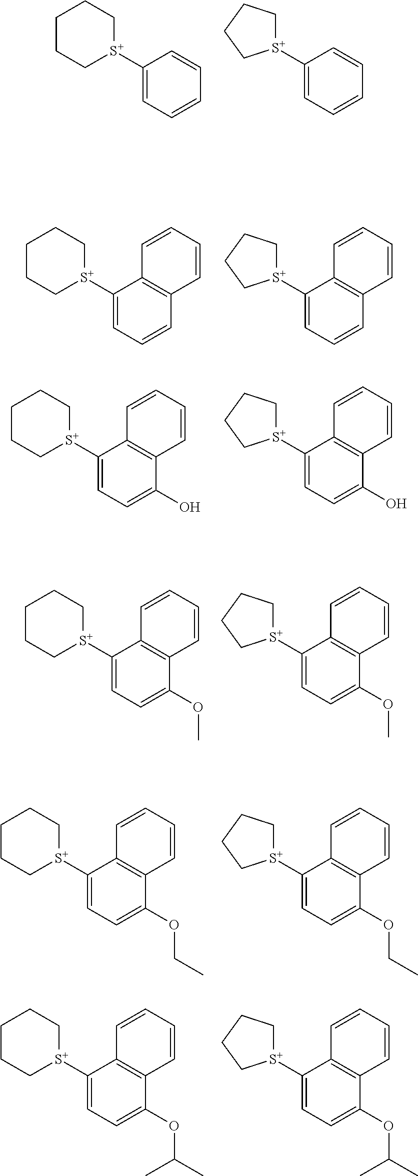

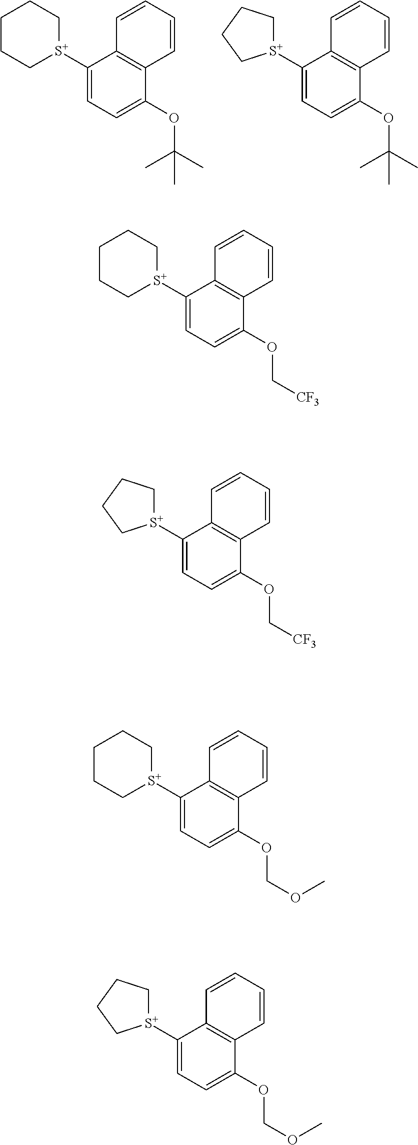

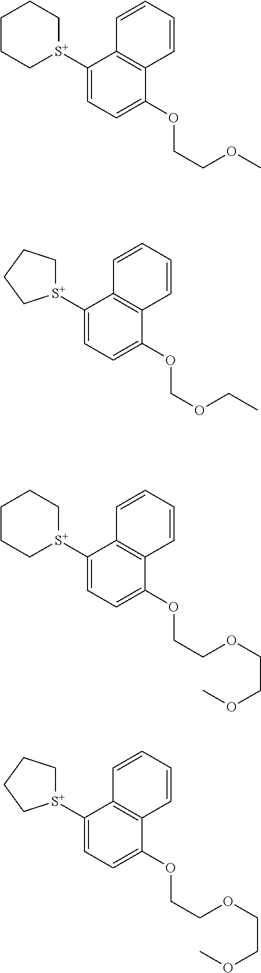

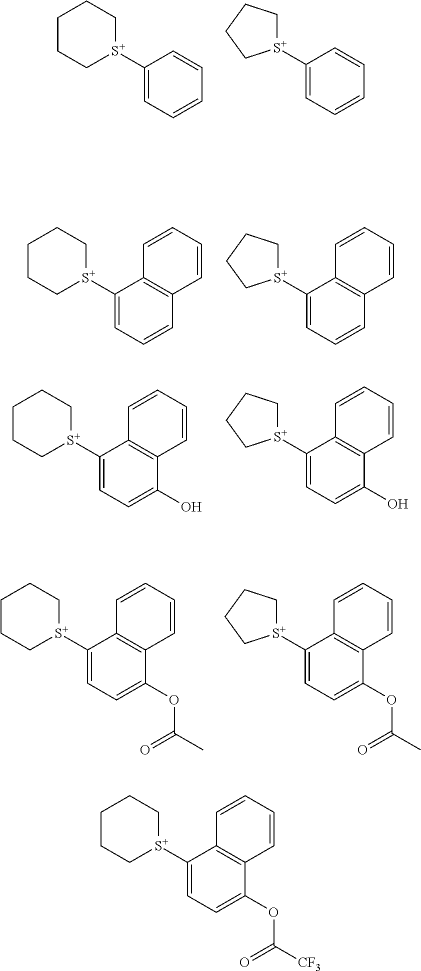

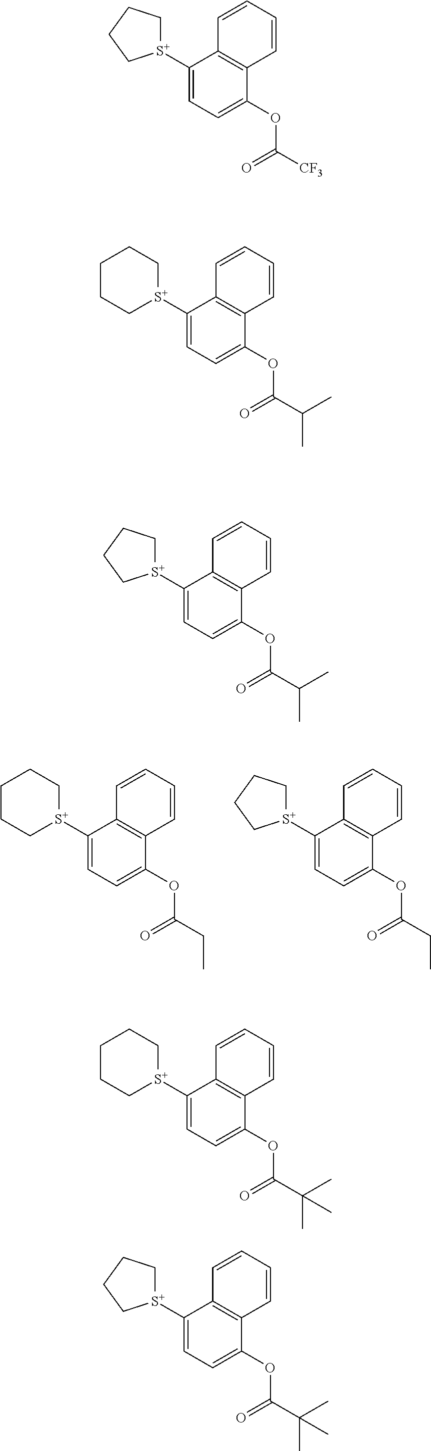

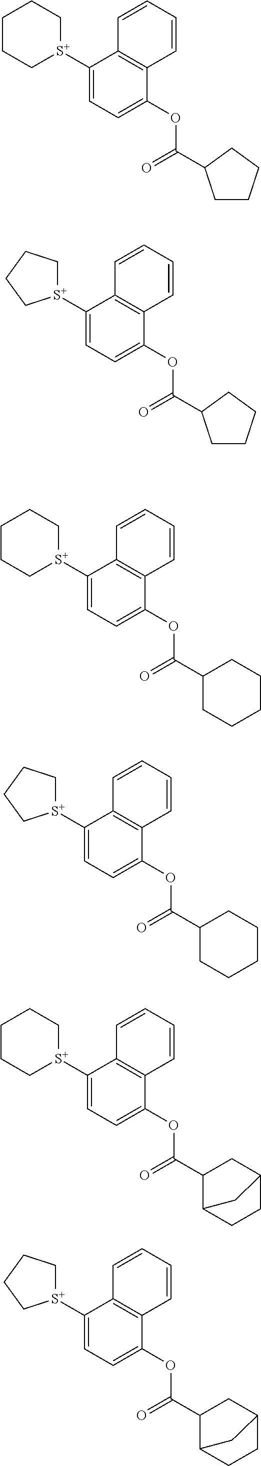

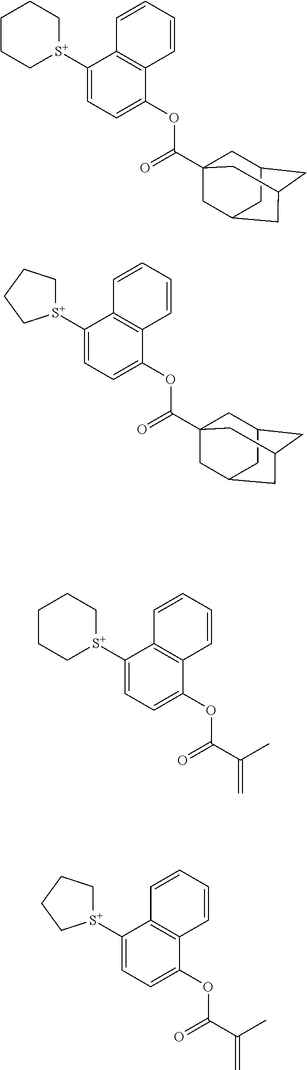

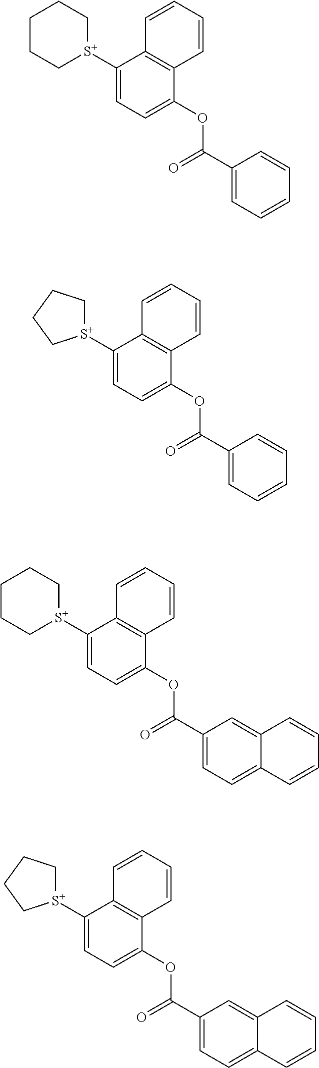

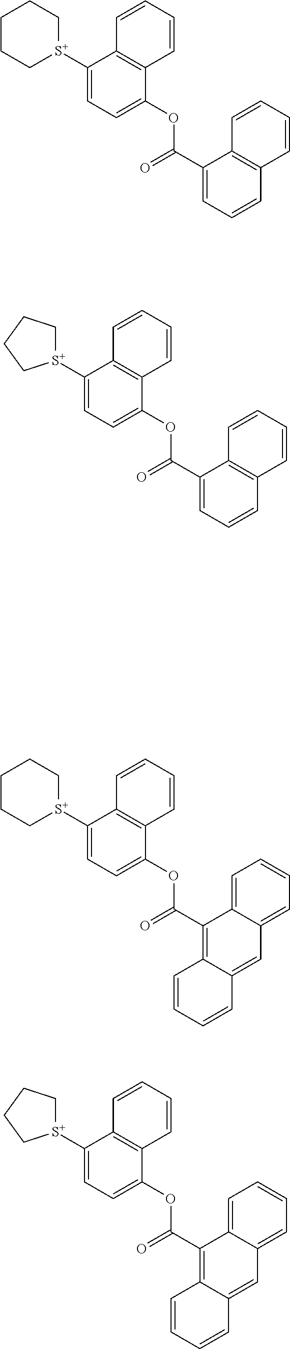

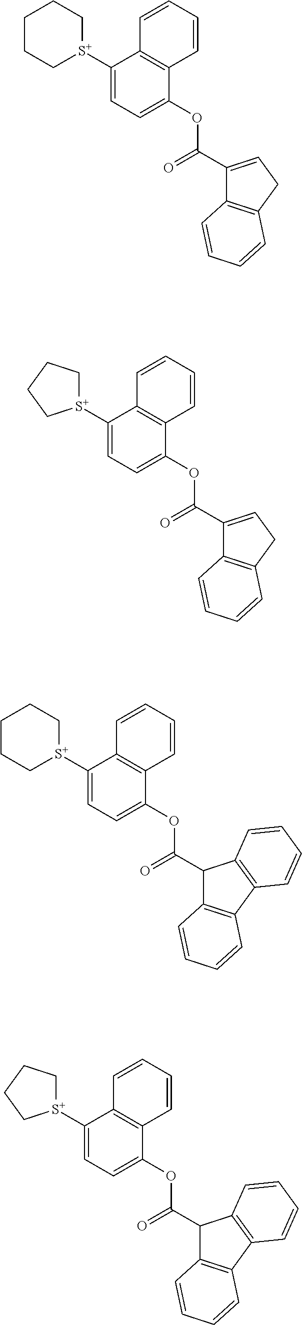

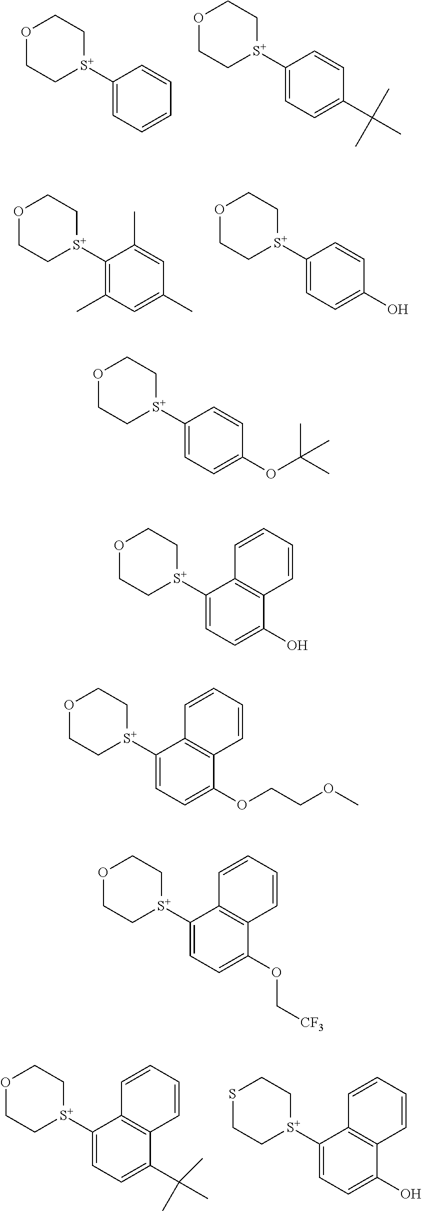

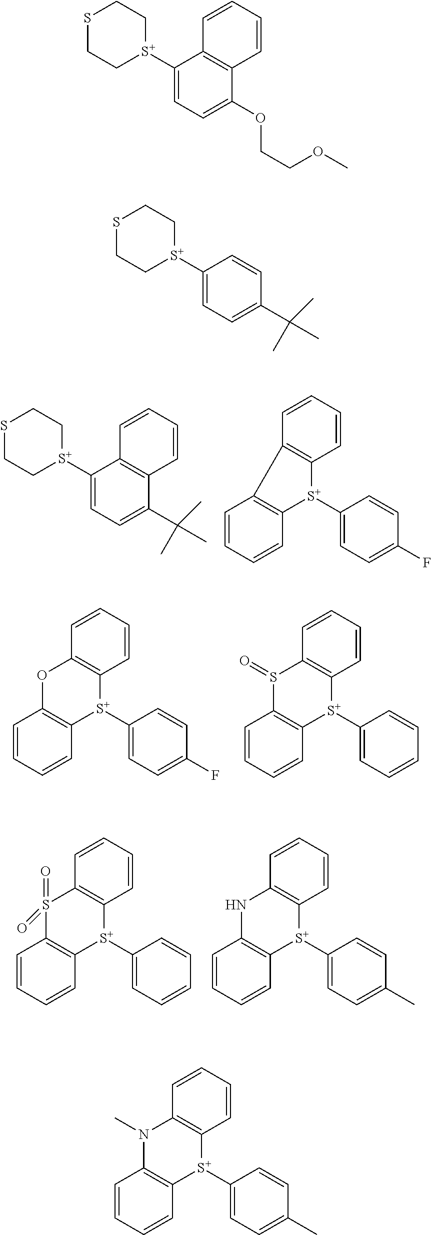

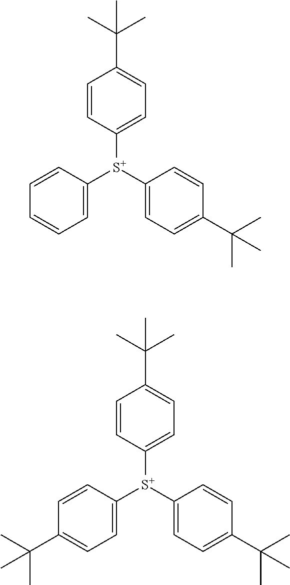

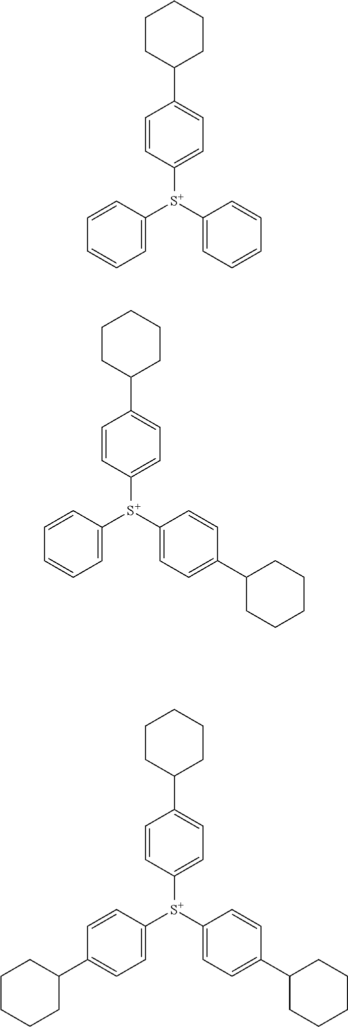

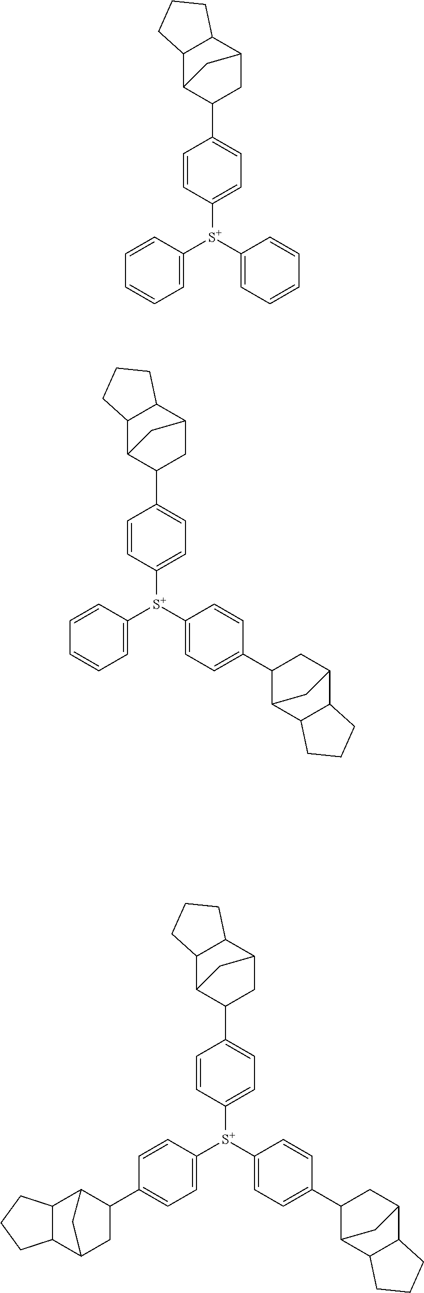

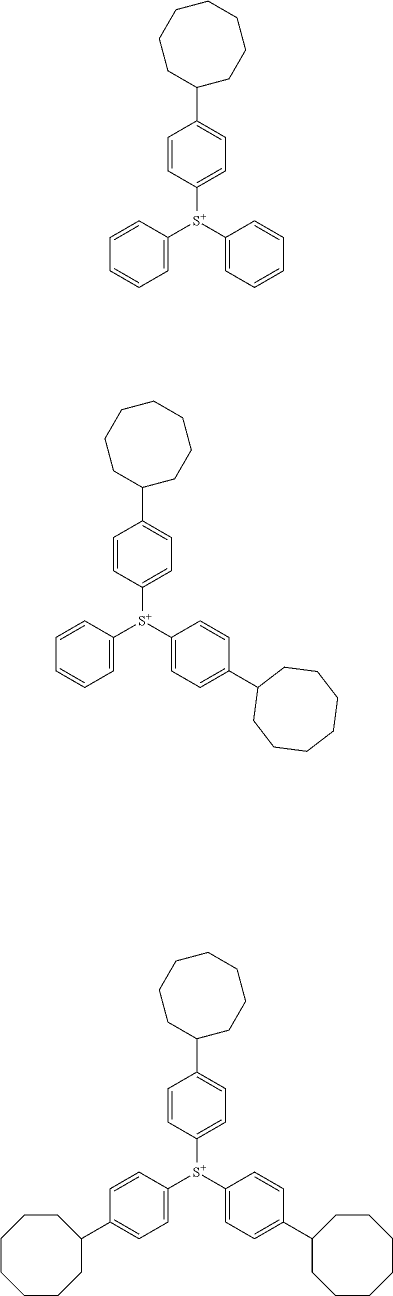

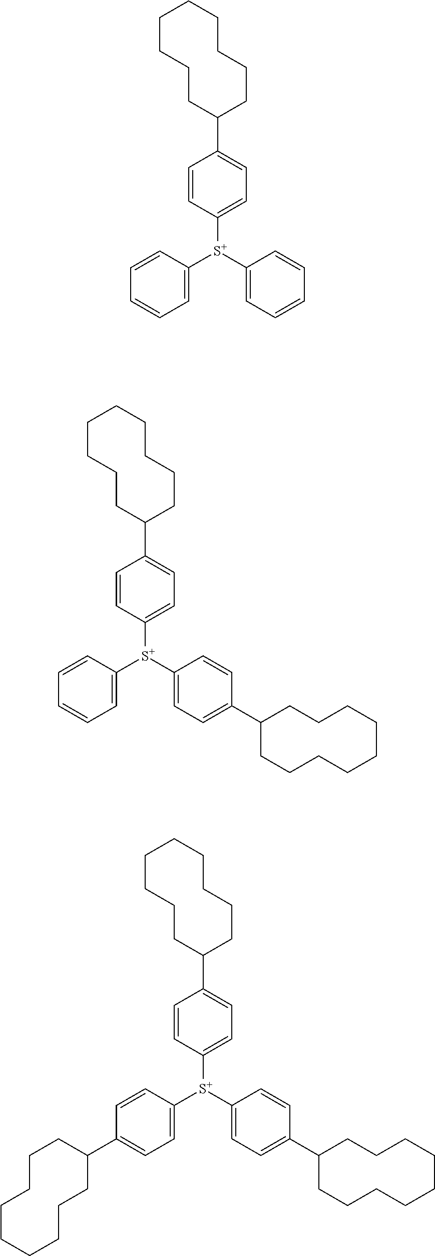

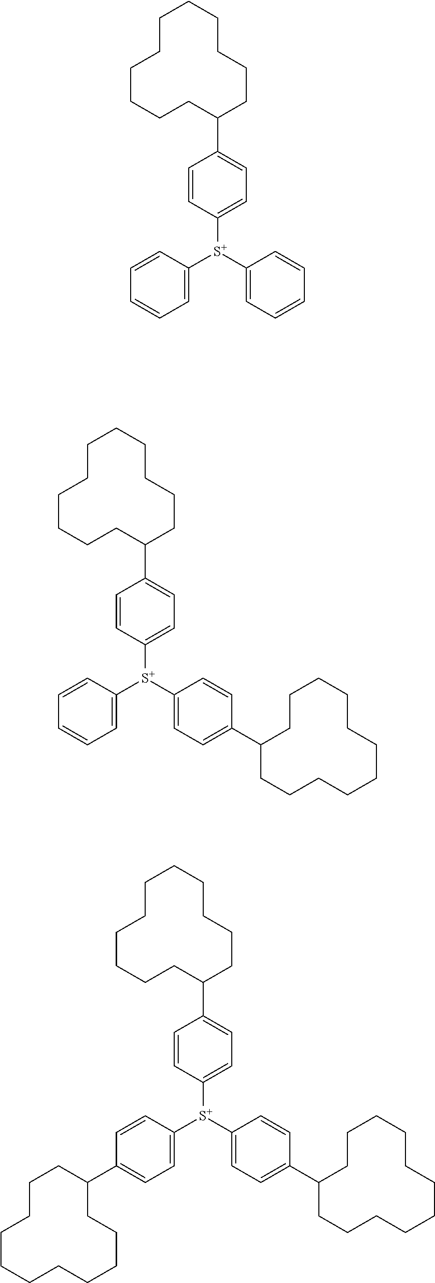

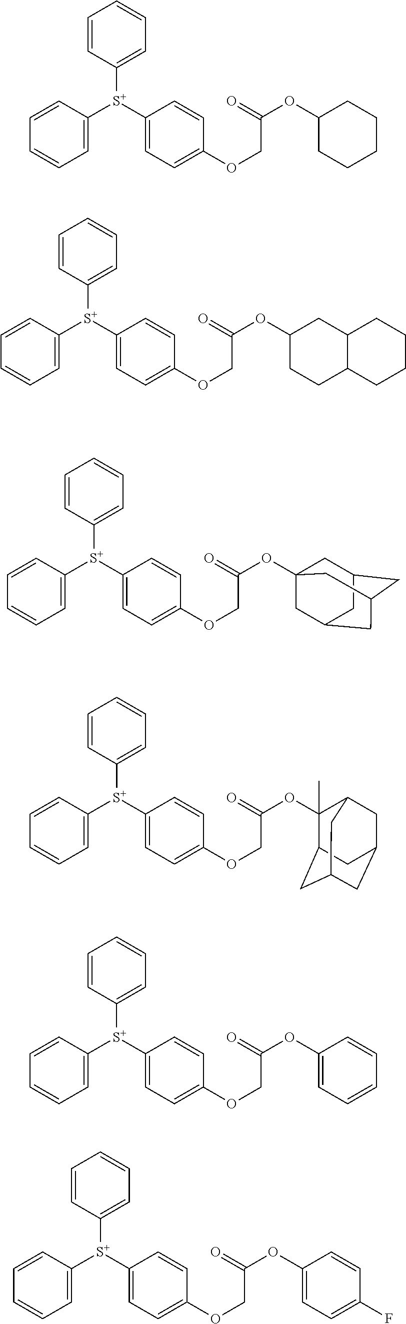

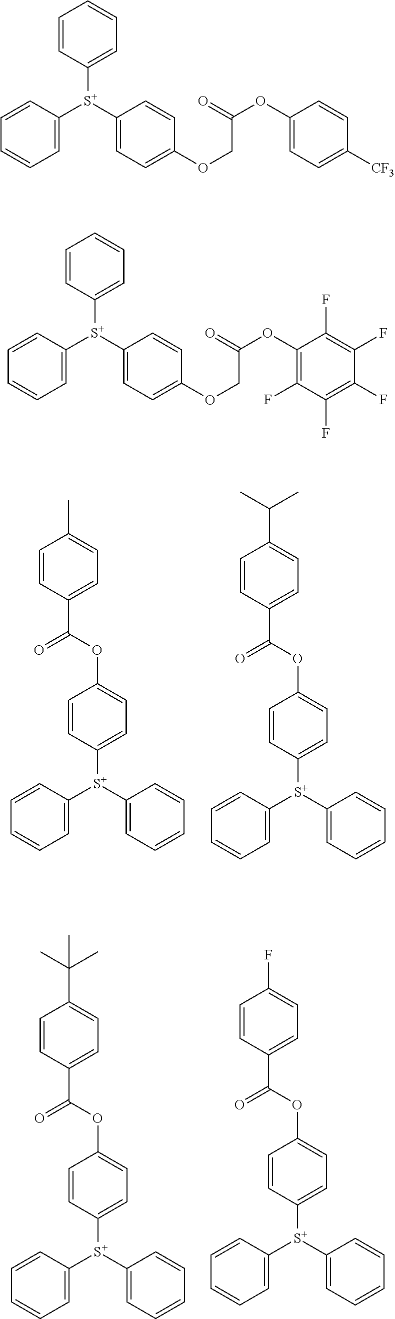

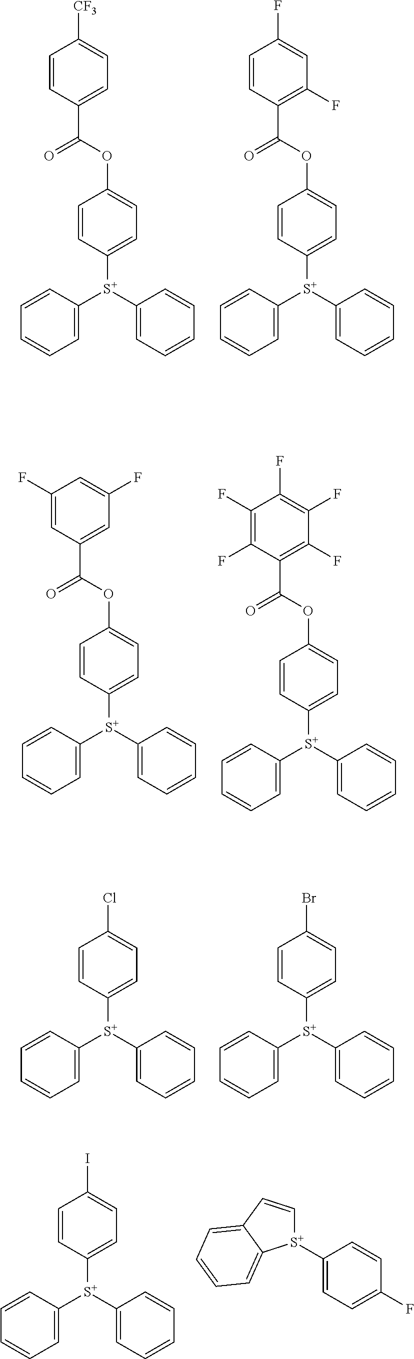

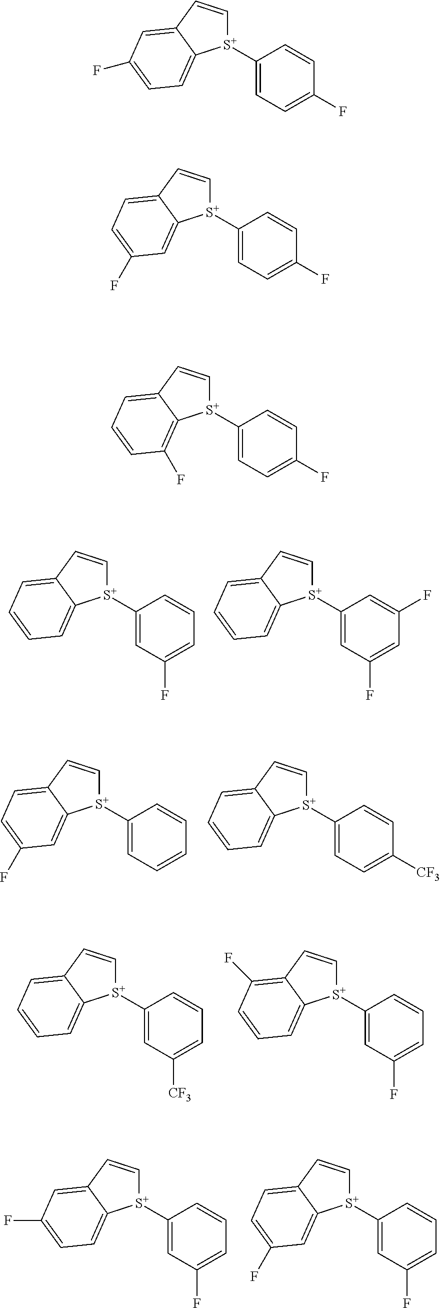

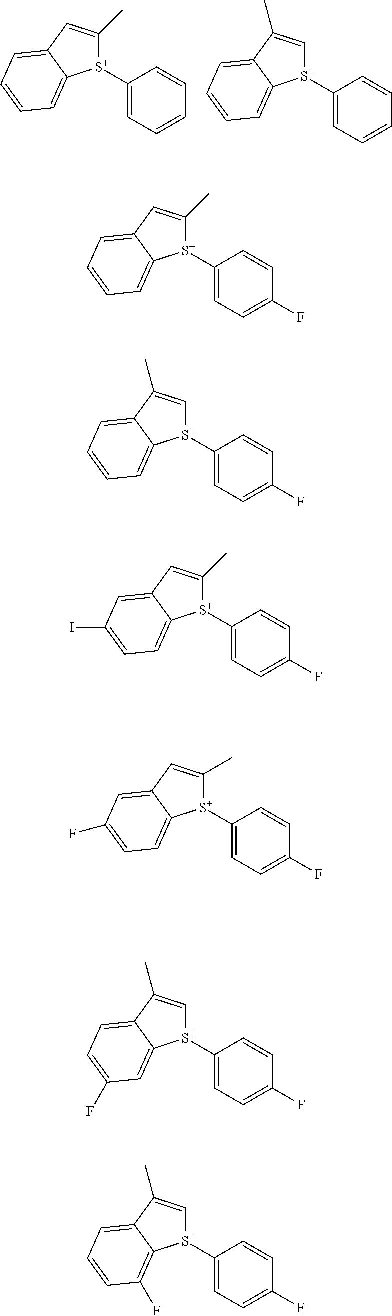

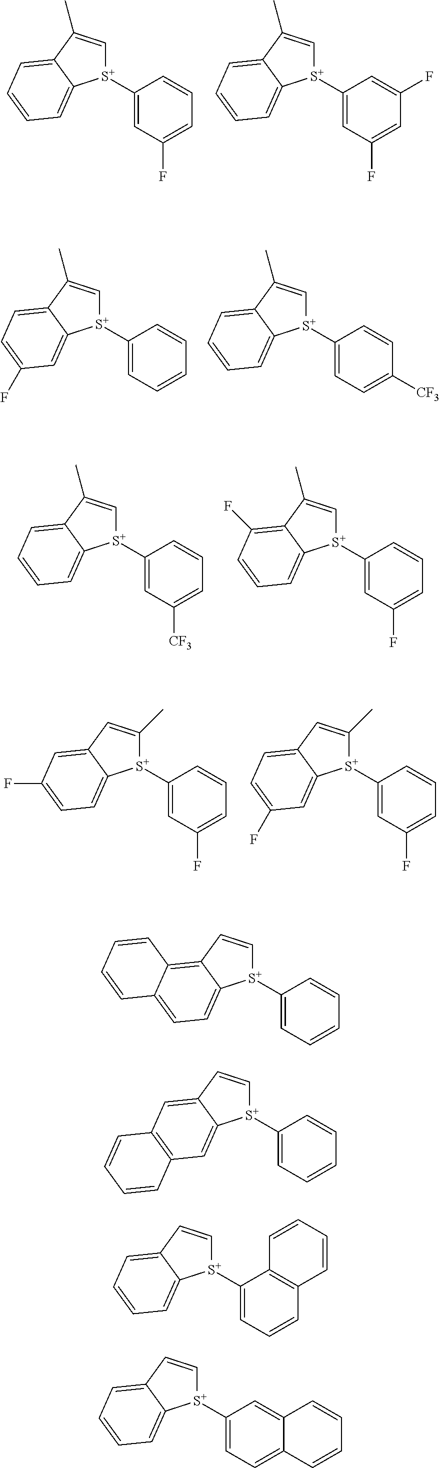

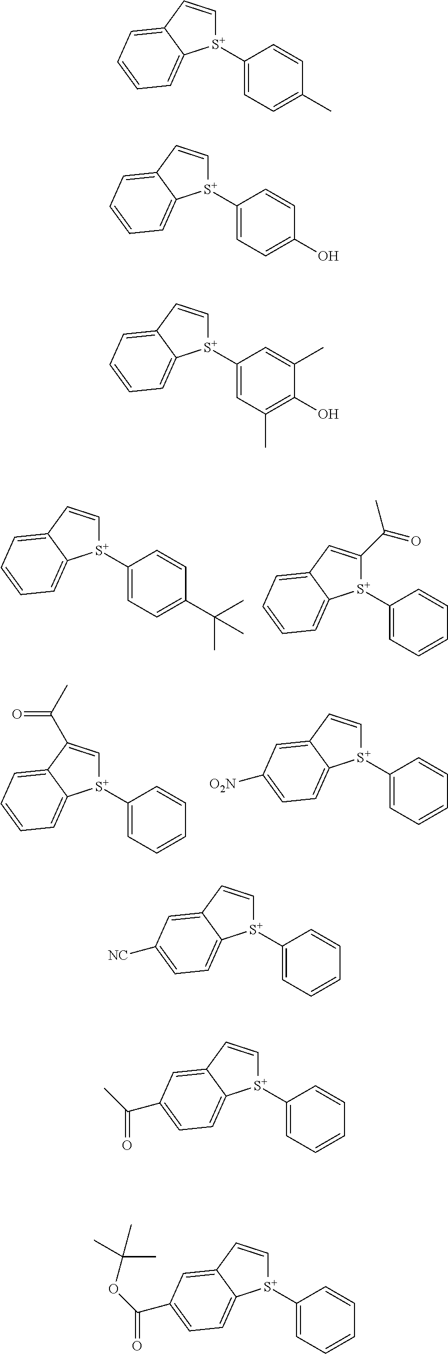

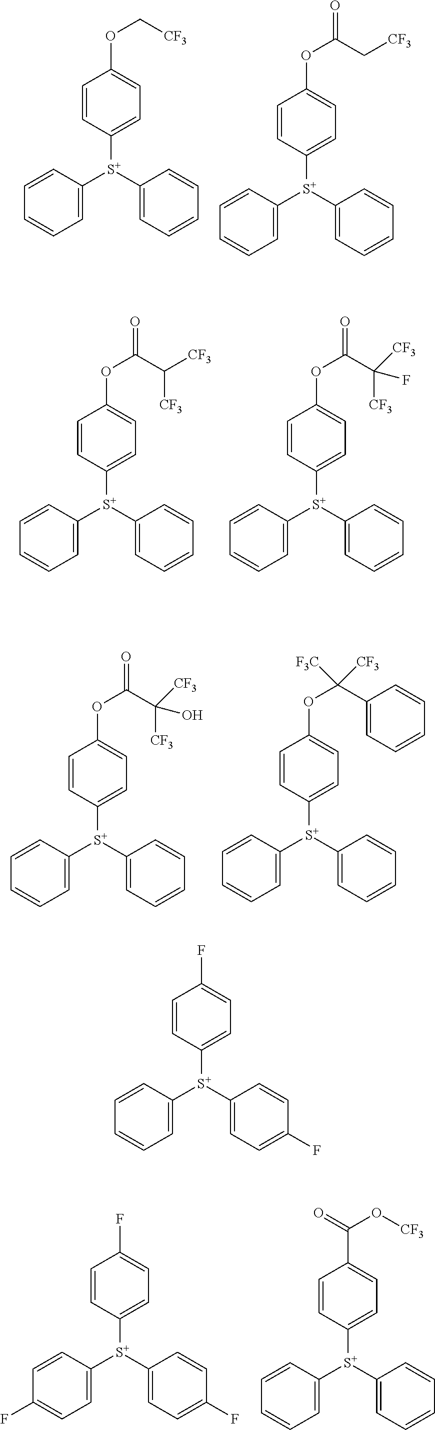

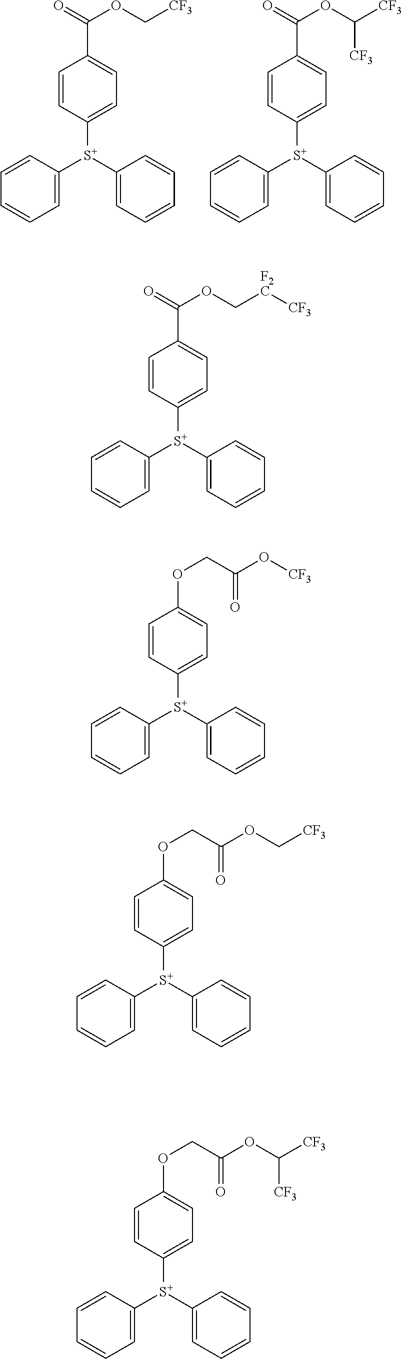

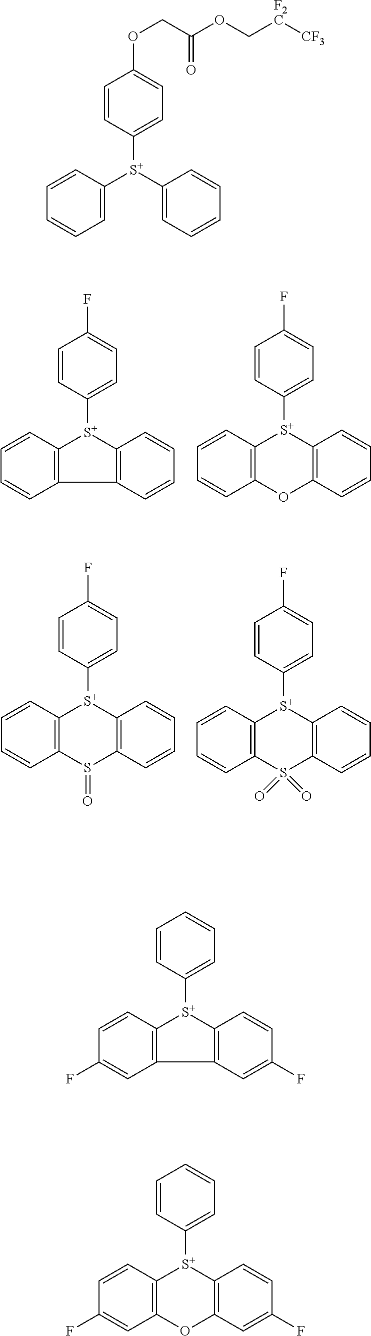

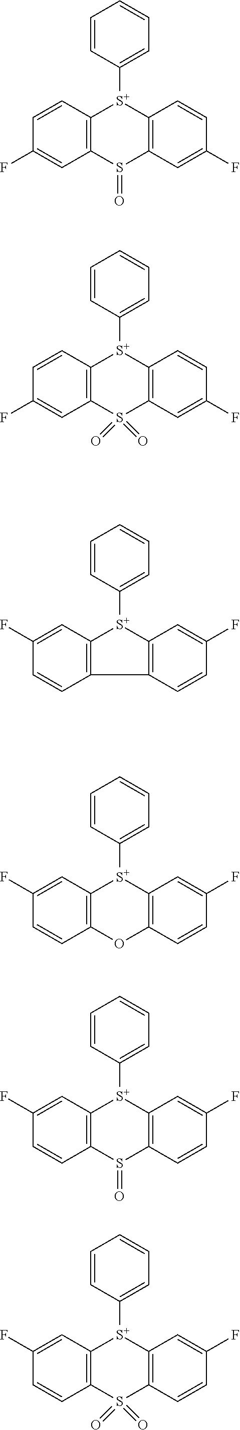

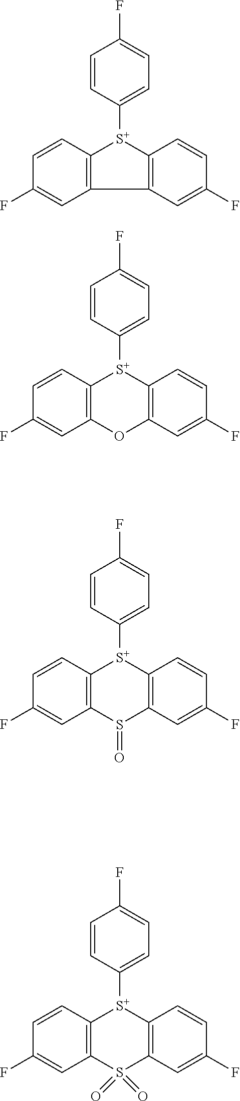

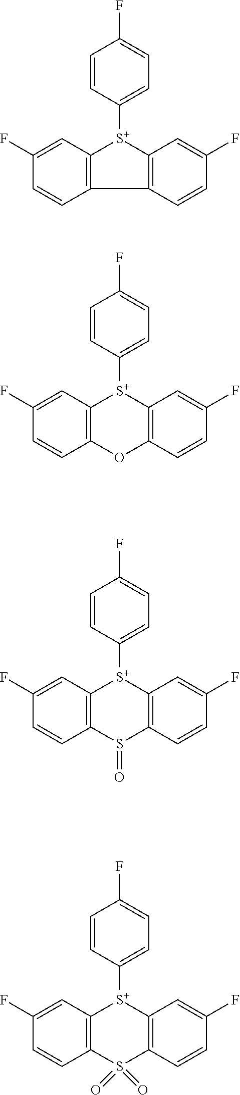

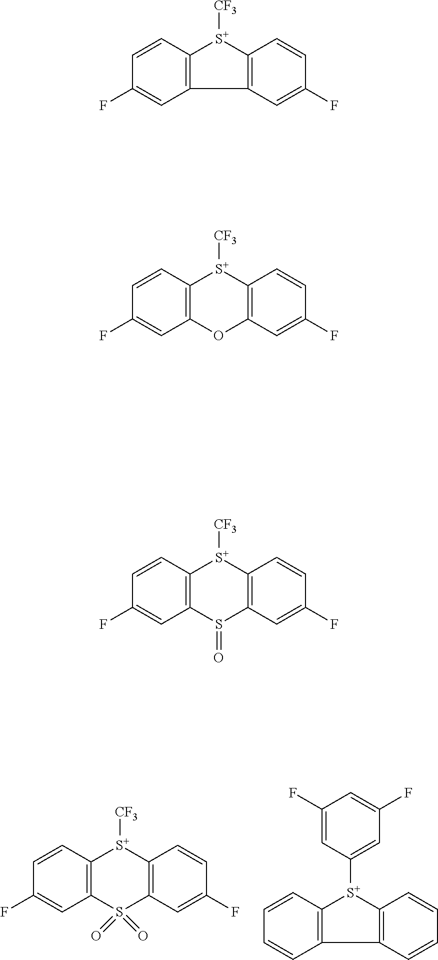

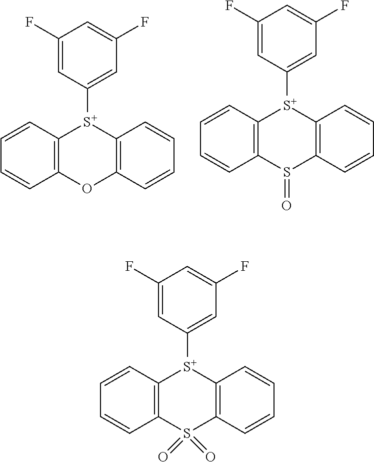

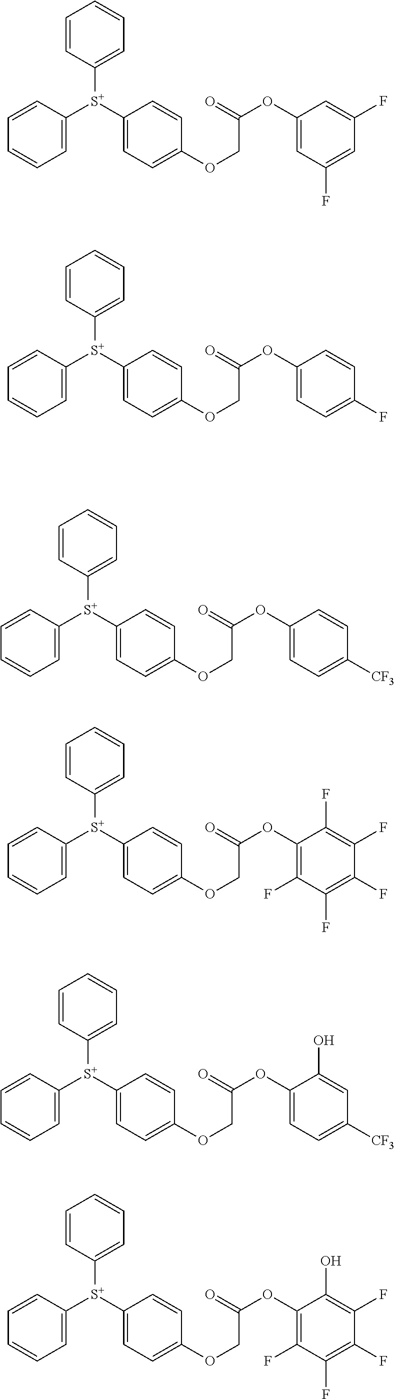

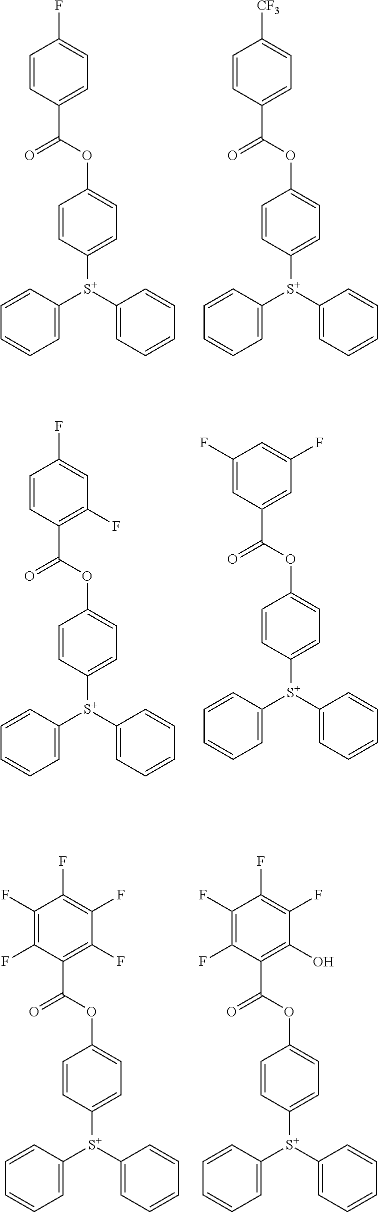

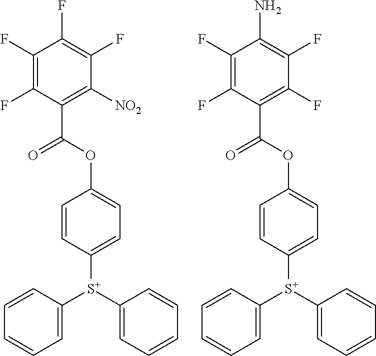

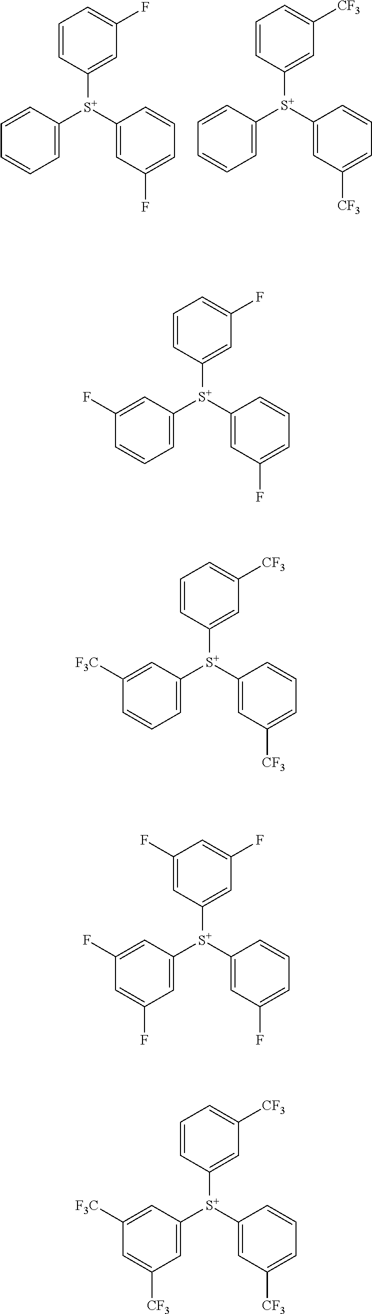

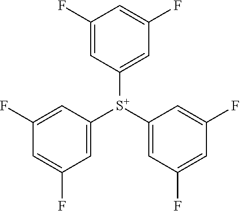

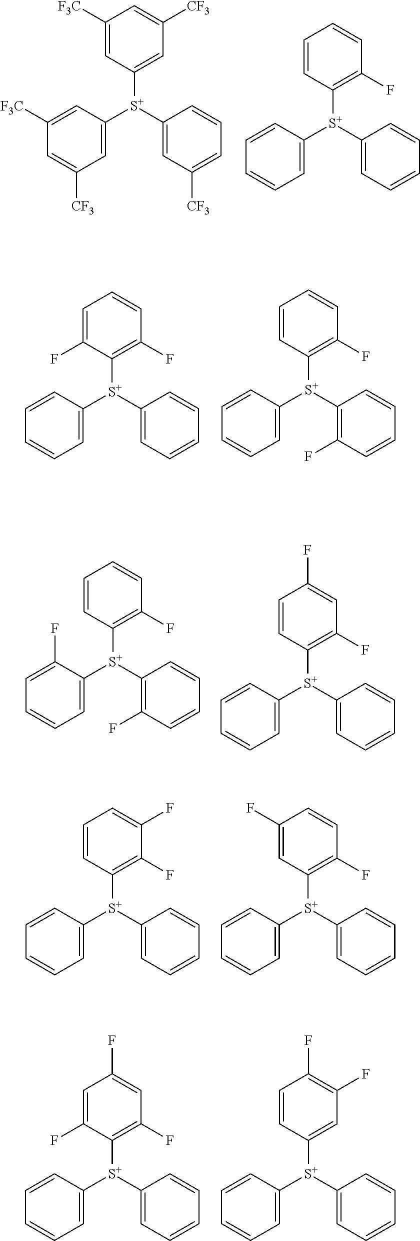

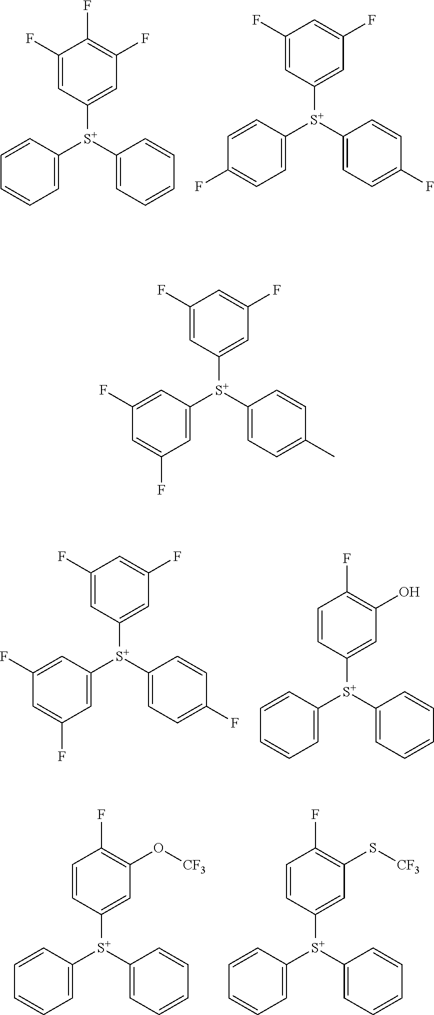

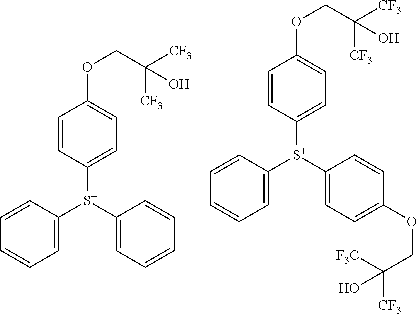

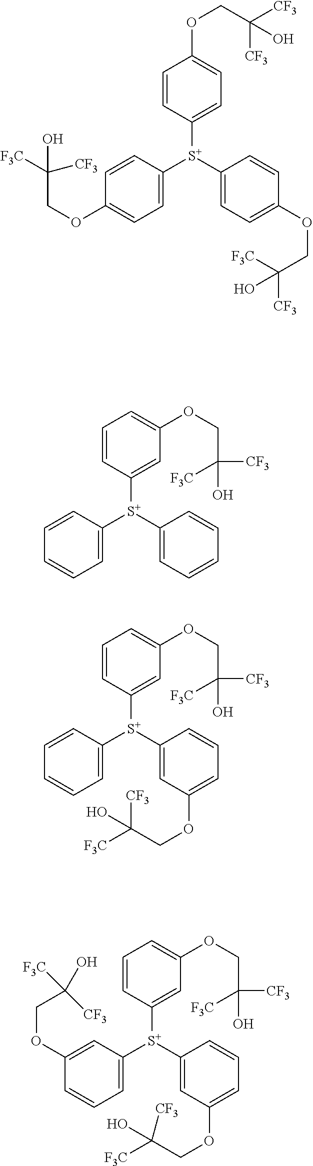

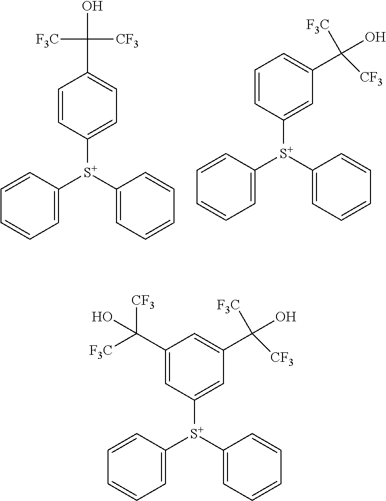

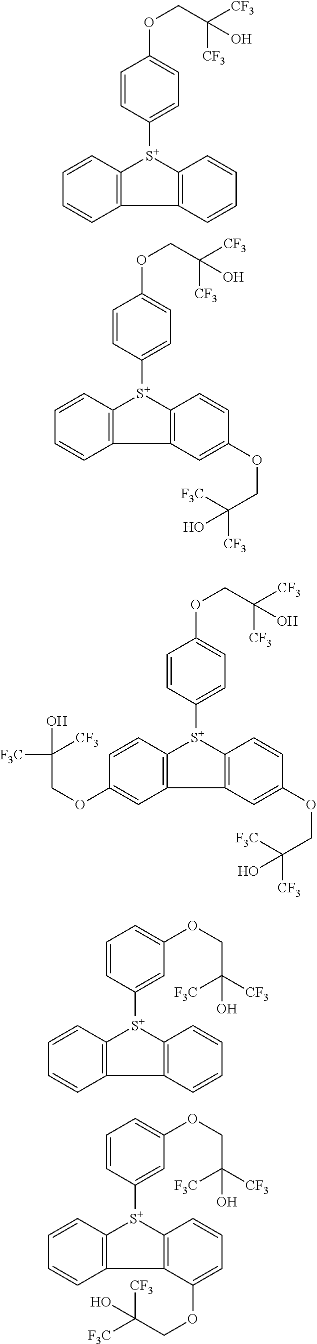

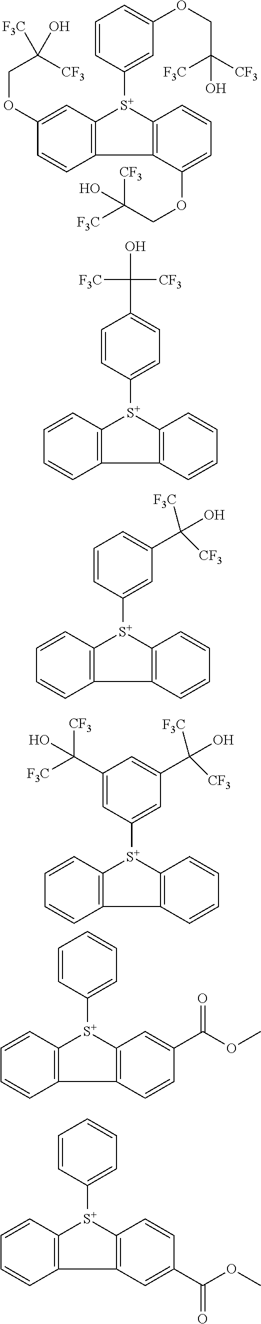

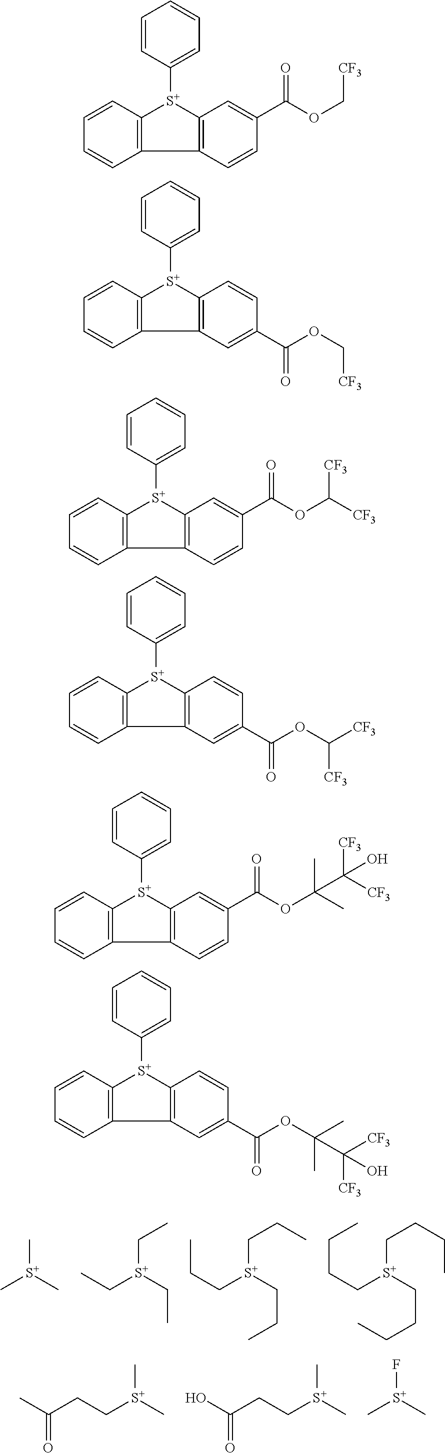

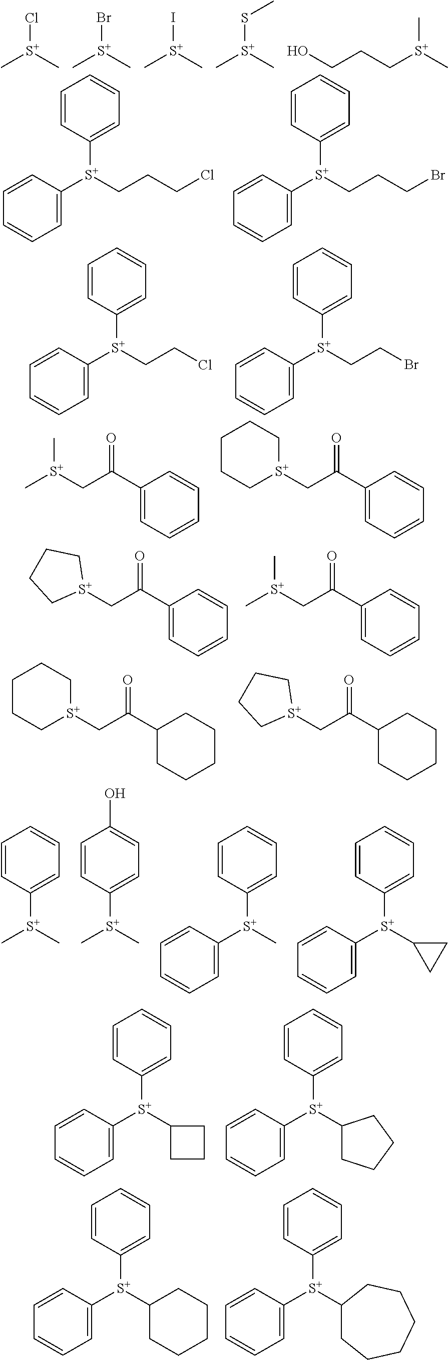

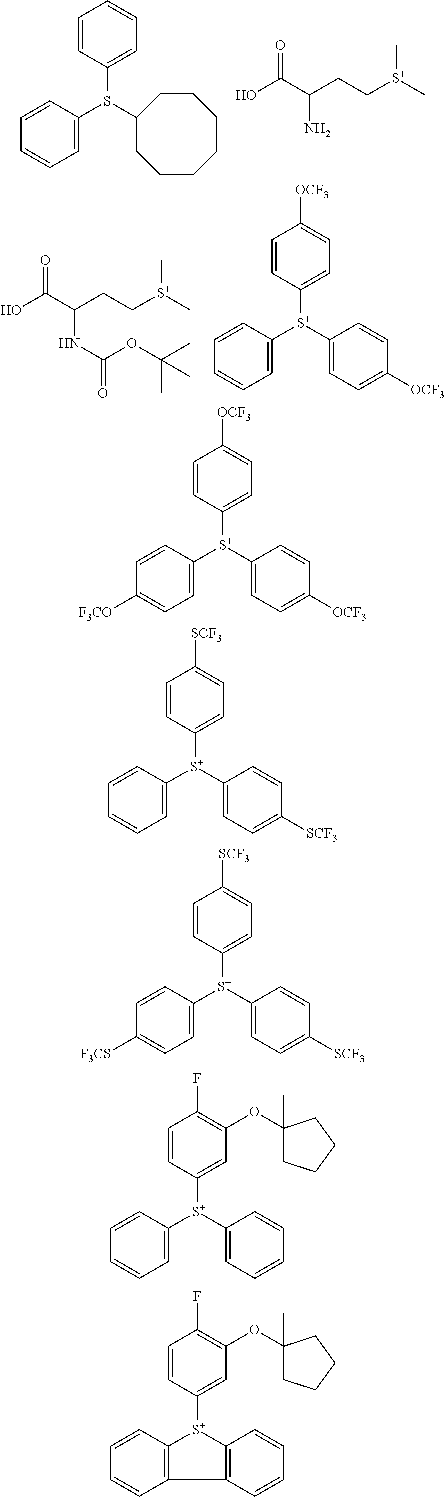

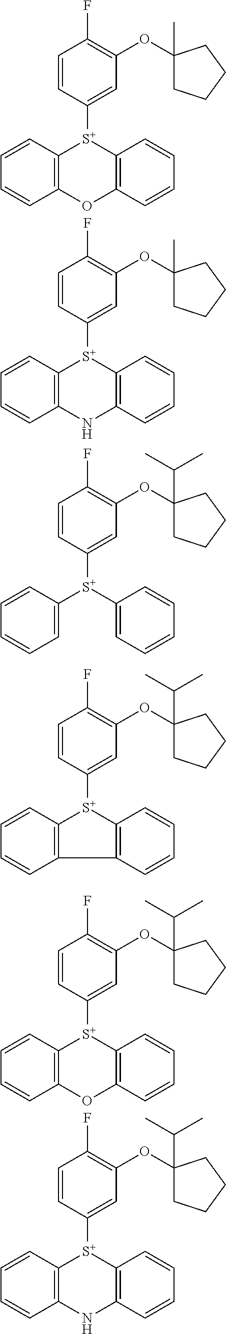

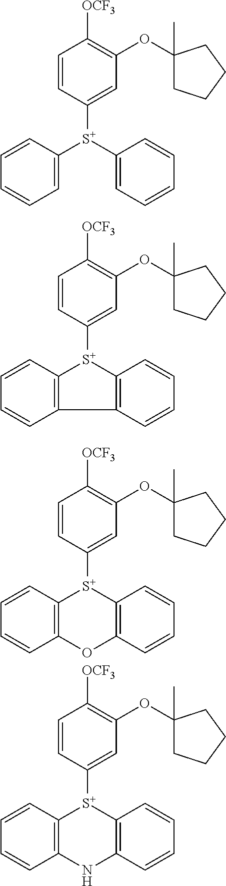

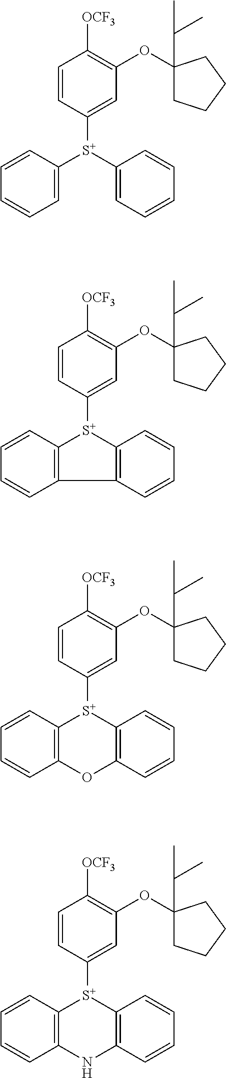

In formula (B3), R²⁶, R²⁷ and R²⁸ are each independently halogen, a C₁-C₂₀ hydrocarbyl group which may contain a heteroatom, or a C₁-C₂₀ hydrocarbyloxy group which may contain a heteroatom. Exemplary of the halogen are fluorine, chlorine, bromine and iodine.

The hydrocarbyl group and hydrocarbyl moiety of the hydrocarbyloxy group may be saturated or unsaturated and straight, branched or cyclic. Examples thereof include C₁-C₂₀ alkyl groups such as methyl, ethyl, n-propyl, isopropyl, n-butyl, sec-butyl, tert-butyl, n-pentyl, tert-pentyl, n-hexyl, n-octyl, 2-ethylhexyl, n-nonyl and n-decyl; and C₃-C₂₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl and adamantyl. In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—), or haloalkyl moiety. R²⁶, R²⁷ and R²⁸ are preferably selected from methyl, methoxy, tert-butyl and tert-butoxy.

In formula (B3), p and q are each independently 0, 1, 2, 3, 4 or 5 and r is 0, 1, 2, 3 or 4.

In formula (B3), L is a single bond, ether bond or a C₁-C₂₀ hydrocarbylene group which may contain a heteroatom. Examples of the hydrocarbylene group are as exemplified above for hydrocarbylene group RN³. In the hydrocarbylene groups, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—), or haloalkyl moiety.

Examples of the photoacid generator having formula (B3) are shown below, but not limited thereto. Herein A² is as defined above.

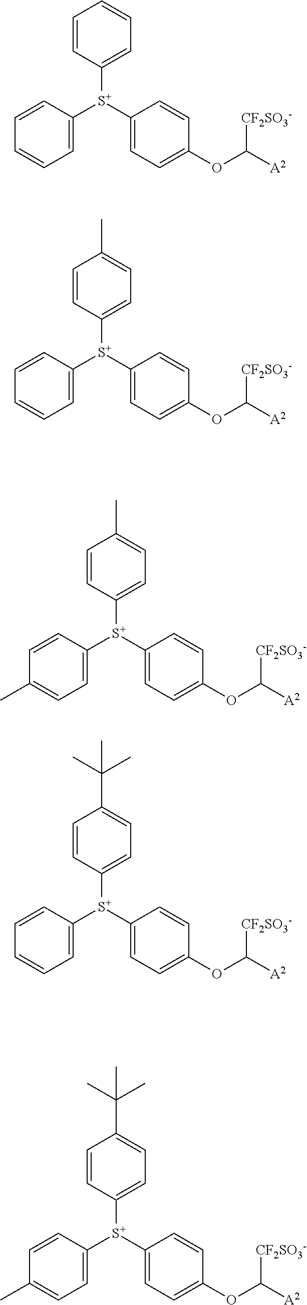

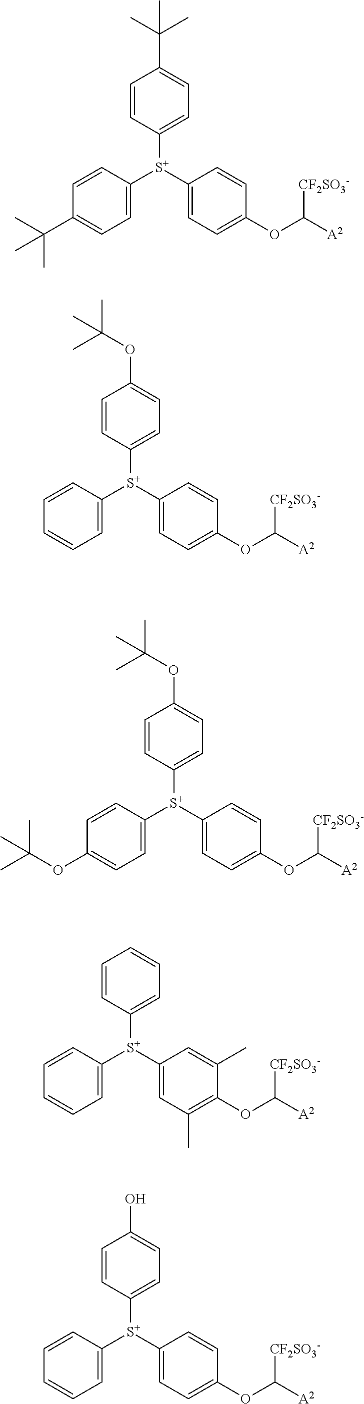

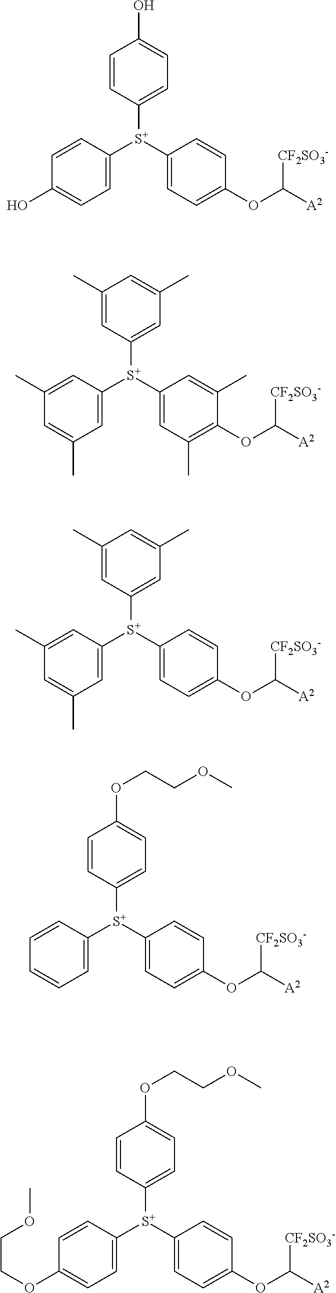

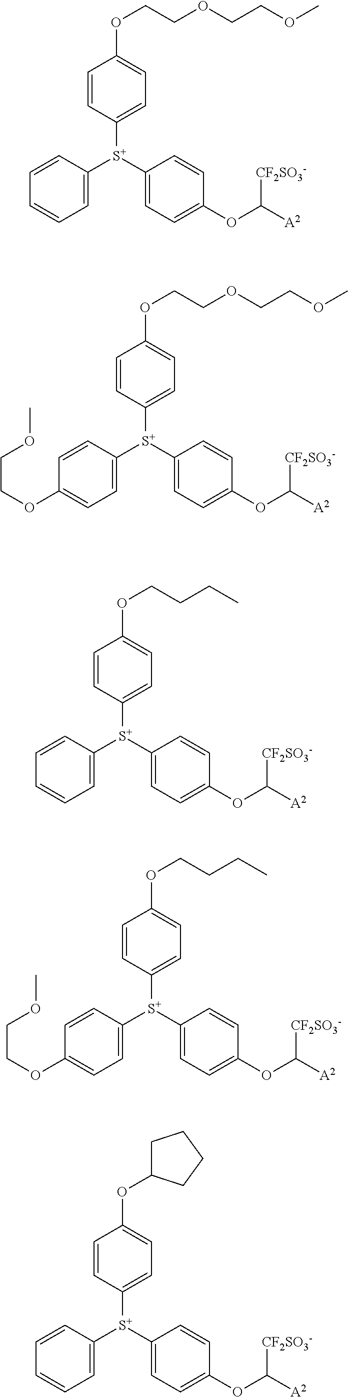

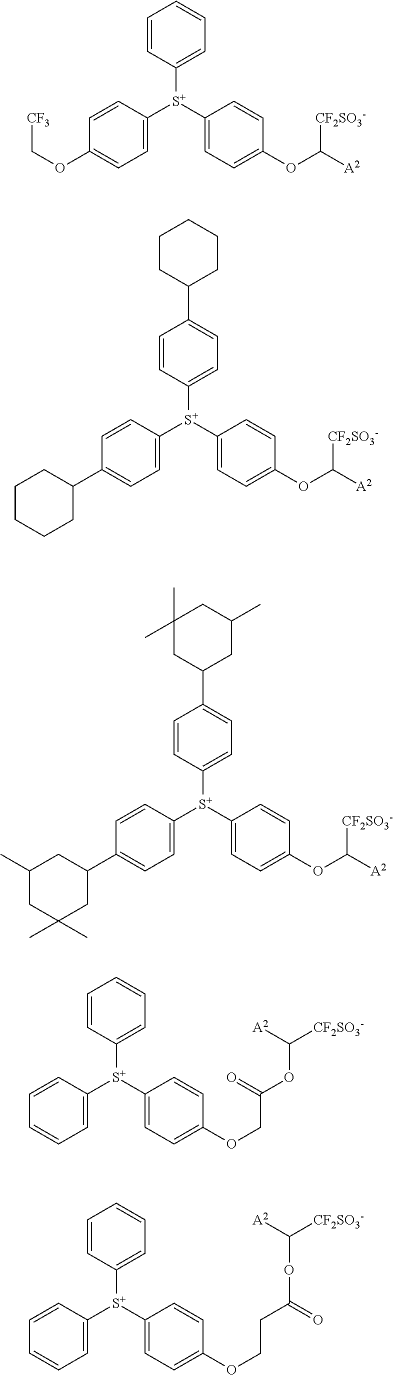

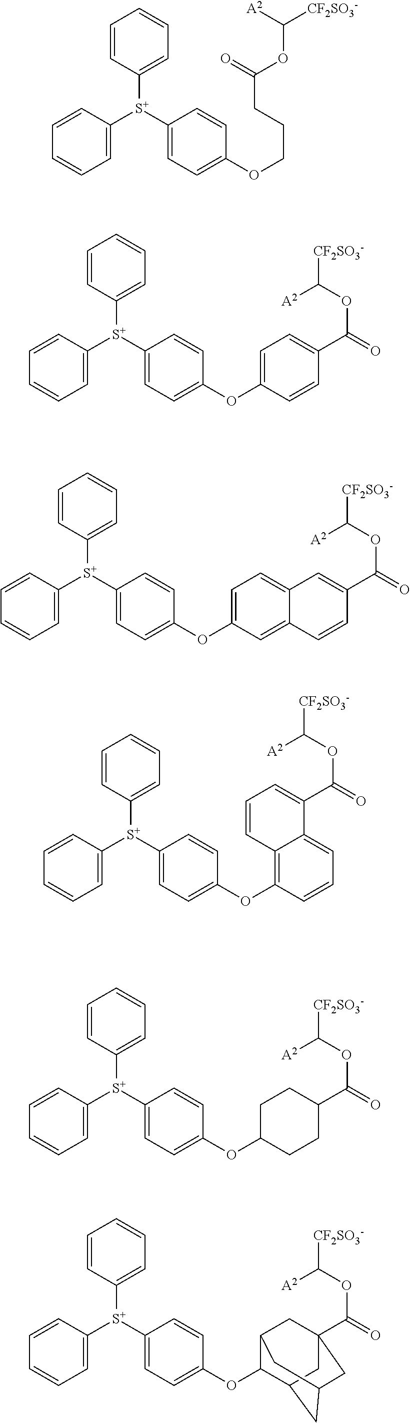

In the chemically amplified resist composition, the PAG is preferably added in an amount of 0.1 to 40 parts, more preferably 0.1 to 20 parts by weight per 80 parts by weight of the base polymer. As long as the amount of the PAG is in the range, the risk that resolution is degraded and foreign particles are left on the pattern after development or stripping of the resist is eliminated.

The chemically amplified resist composition may contain a photoacid generator other than the above-mentioned photoacid generator for the purpose of fine adjustment of lithography properties. The other PAG may be any of well-known ones used in conventional chemically amplified resist compositions. Examples of the other PAG include sulfonium salt, iodonium salt, sulfonyldiazomethane, N-sulfonyloxyimide, and oxime-O-sulfonate type acid generators, which may be used alone or in admixture. The other PAGs capable of generating a strong acid such as sulfonic acid, (bisperfluoroalkanesulfonyl) imide and (trisperfluoromethanesulfonyl) methide or weak acid such as carboxylic acid are preferred.

Illustrative examples of the other PAG are described in JP-A 2008-111103, paragraphs [0122]-[0142]. Preference is given to the compounds described in JP-A 2014-001259, paragraphs [0088]-[0092], JP-A 2012-041320, paragraphs [0015]-[0017], and JP-A 2012-106986, paragraphs [0015]-[0029]. These PAGs capable of generating partially fluorinated sulfonic acids are advantageously used in the ArF lithography and ArF immersion lithography because the generated acid has an appropriate strength and diffusion length.

When the chemically amplified resist composition contains the other PAG, the amount thereof is preferably 0.1 to 50 parts by weight, more preferably 0.2 to 40 parts by weight, even more preferably 0.3 to 35 parts by weight per 80 parts by weight of the base polymer. As long as the amount of the other PAG is in the range, the risk that resolution is degraded and foreign particles are left on the pattern after development or stripping of the resist is eliminated. It is noted that when the compound having formula (B2) is used, its amount is preferably 0 to 50% by weight based on the total of the base polymer and PAG(s).

### <Organic Solvent>

The chemically amplified resist composition typically contains an organic solvent. The organic solvents used herein are described in JP-A 2008-111103, paragraphs [0144]-[0145]. Exemplary solvents include ketones such as cyclohexanone, cyclopentanone and methyl-2-n-pentyl ketone; alcohols such as 3-methoxybutanol, 3-methyl-3-methoxybutanol, 1-methoxy-2-propanol, 1-ethoxy-2-propanol and diacetone alcohol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as γ-butyrolactone (GBL); and mixtures thereof. When acid labile groups of acetal type are used, high-boiling alcohols may be added to accelerate deprotection reaction of acetal. For example, diethylene glycol, propylene glycol, glycerin, 1,4-butanediol or 1,3-butanediol may be added.

The amount of the organic solvent is preferably 100 to 10,000 parts by weight, more preferably 300 to 8,000 parts by weight per 80 parts by weight of the base polymer. The organic solvent may be used alone or in admixture.

### <Fluorinated Polymer>

The chemically amplified resist composition may further comprise a fluorinated polymer comprising repeat units of at least one type selected from repeat units having the formula (D1), repeat units having the formula (D2), and repeat units having the formula (D3). Notably, the fluorinated polymer is different from the base polymer.

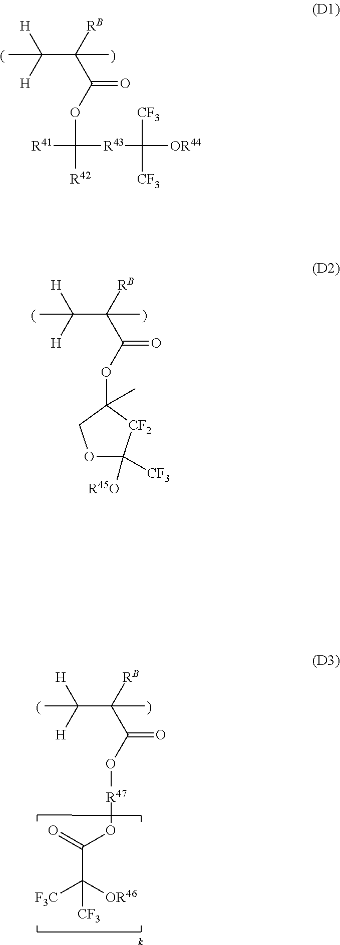

In formulae (D1) to (D3), R^{B} is hydrogen or methyl. R⁴¹ and R⁴² are each independently hydrogen or a C₁-C₁₀ hydrocarbyl group. R⁴³ is a single bond or a C₁-C₅ straight or branched hydrocarbylene group. R⁴⁴, R⁴⁵ and R⁴⁶ are each independently hydrogen or a C₁-C₁₅ hydrocarbyl group, C₁-C₁₅ fluorinated hydrocarbyl group, C₂-C₁₅ acyl group or acid labile group. In the hydrocarbyl or fluorinated hydrocarbyl group represented by R⁴⁴, R⁴⁵ and R⁴⁶, some —CH₂— may be replaced by an ether bond or carbonyl group. R⁴⁷ is a C₁-C₂₀ (k+1)-valent hydrocarbon group or C₁-C₂₀ (k+1)-valent fluorinated hydrocarbon group, and k is 1, 2 or 3.

The C₁-C₁₀ hydrocarbyl group represented by R⁴¹ and R⁴² is preferably saturated and may be straight, branched or cyclic. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl, and n-decyl, and C₃-C₁₀ cyclic saturated hydrocarbyl groups such as cyclopropyl, cyclobutyl, cyclopentyl, cyclohexyl, adamantyl, and norbornyl. Inter alia, C₁-C₆ saturated hydrocarbyl groups are preferred.

The C₁-C₅ hydrocarbylene group represented by R⁴³ is preferably saturated and may be straight, branched or cyclic. Examples thereof include methylene, ethylene, trimethylene, propylene, tetramethylene, and pentamethylene.

The C₁-C₁₅ hydrocarbyl group represented by R⁴⁴, R⁴⁵ and R⁴⁶ may be saturated or unsaturated and straight, branched or cyclic. Examples thereof include saturated hydrocarbyl groups and aliphatic unsaturated hydrocarbyl groups such as alkenyl and alkynyl groups. The saturated hydrocarbyl groups are preferred, and examples thereof include those exemplified for the C₁-C₁₀ saturated hydrocarbyl group R⁴¹ and R⁴² and n-undecyl, n-dodecyl, tridecyl, tetradecyl, and pentadecyl. The C₁-C₁₅ fluorinated hydrocarbyl groups correspond to the foregoing hydrocarbyl groups in which some or all carbon-bonded hydrogen atoms are substituted by fluorine atoms. In the hydrocarbyl and fluorinated hydrocarbyl groups, some —CH₂— may be replaced by an ether bond or carbonyl moiety.

When R⁴⁴, R⁴⁵ and R⁴⁶ each are an acid labile group, examples of the acid labile group include groups having formulae (a-1) to (a-10), C₄-C₂₀, especially C₄-C₁₅ tertiary hydrocarbyl groups, trihydrocarbylsilyl groups in which each hydrocarbyl moiety is of 1 to 6 carbon atoms, and C₄-C₂₀ hydrocarbyl groups containing a carbonyl moiety, ether bond or ester bond.

Examples of the C₁-C₂₀ (k+1)-valent hydrocarbon and fluorinated hydrocarbon groups R⁴⁷ include the foregoing hydrocarbyl and fluorinated hydrocarbyl groups from which number “k” of hydrogen atoms are eliminated.

Examples of the repeat unit having formula (D1) are given below, but not limited thereto. Herein R^{B} is as defined above.

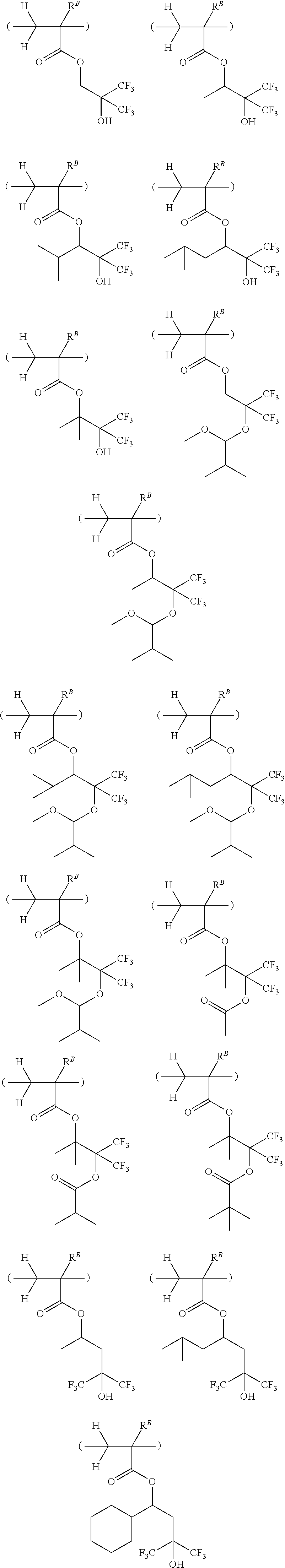

Examples of the repeat unit having formula (D2) are given below, but not limited thereto. Herein R^{B} is as defined above.

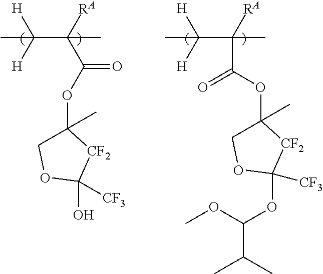

Examples of the repeat unit having formula (D3) are given below, but not limited thereto. Herein R^{B} is as defined above.

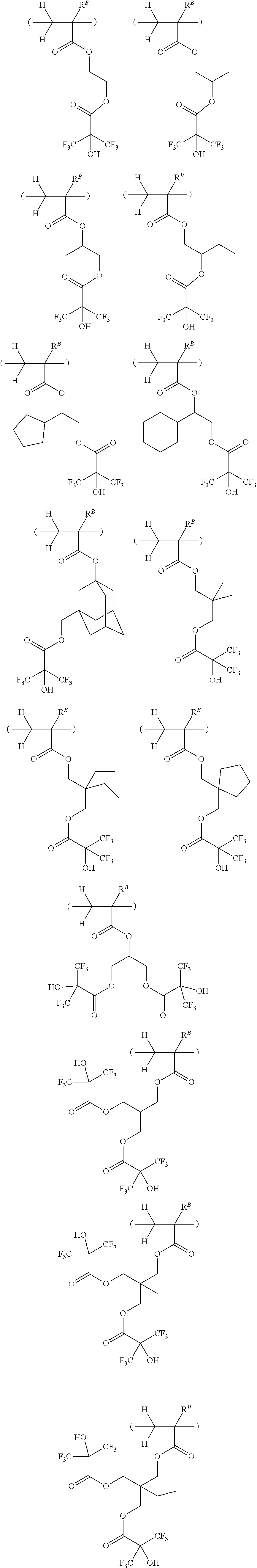

The fluorinated polymer may comprise additional repeat units other than the repeat units having formulae (D1) to (D3). Examples of the additional repeat units include repeat units obtained from methacrylic acid and α-trifluoromethylacrylic acid derivatives. The content of repeat units having formulae (D1) to (D3) is preferably at least 20 mol %, more preferably at least 60 mol %, most preferably 100 mol % based on the overall repeat units.

The fluorinated polymer should preferably have a Mw of 1,000 to 100,000, and more preferably 3,000 to 15,000 and a dispersity (Mw/Mn) of 1.0 to 2.0, more preferably 1.0 to 1.6.

The fluorinated polymer may be synthesized by combining one or more monomers providing at least one of repeat units having formula (D1), (D2) and (D3) and optionally one or more monomers providing additional repeat units in an organic solvent, adding a radical initiator, and heating for polymerization.

Examples of the organic solvent which can be used for polymerization include toluene, benzene, THF, diethyl ether, dioxane, methyl ethyl ketone (MEK), propylene glycol monomethyl ether (PGME), propylene glycol monomethyl ether acetate (PGMEA). Examples of the polymerization initiator used herein include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. The initiator is preferably added in an amount of 0.01 to 25 mol % based on the total of monomers to be polymerized. The reaction temperature is preferably 50 to 100° C. The reaction time is preferably 4 to 24 hours. The acid labile group that has been incorporated in the monomer may be kept as such, or polymerization may be followed by protection or partial protection. During the polymer synthesis, any known chain transfer agent such as dodecyl mercaptan or 2-mercaptoethanol may be added for molecular weight control purpose. The amount of chain transfer agent added is preferably 0.01 to 10 mol % based on the total of monomers.

When the resist composition contains the fluorinated polymer, the amount thereof is preferably 0.1 to 50 parts by weight, and more preferably 0.5 to 10 parts by weight per 80 parts by weight of the base polymer. An amount of the fluorinated polymer in the range is effective in improving the contact angle with water of the resist film at the surface, eliminating the risk of defect formation by residual immersion water or leaching of acid generator and quencher therewith. The fluorinated polymer is also effective for adjusting the solubility of the resist film on the surface and achieving a satisfactory CDU. The fluorinated polymer may be used alone or in admixture.

### <Quencher>

The chemically amplified resist composition may further comprise a quencher if necessary. As used herein, the “quencher” refers to a compound capable of trapping the acid generated by the PAG to prevent the acid from diffusing into the unexposed region of resist film.

Amine compounds, sulfonic acid salts and carboxylic acid salts may be used as the quencher. Suitable amine compounds include primary, secondary and tertiary amine compounds, specifically amine compounds having a hydroxy group, ether bond, ester bond, lactone ring, cyano group or sulfonate ester bond, as described in JP-A 2008-111103, paragraphs [0146]-[0164](U.S. Pat. No. 7,537,880), and primary or secondary amine compounds protected with a carbamate group, as described in JP 3790649. The protected amine compound is effective particularly when the resist composition contains a base-labile component.

Exemplary of the sulfonic acid salt are compounds having the formula (E1). Exemplary of the carboxylic acid salt are compounds having the formula (E2).

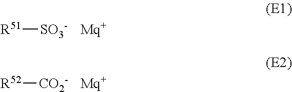

In formula (E1), R⁵¹ is hydrogen or a C₁-C₄₀ hydrocarbyl group which may contain a heteroatom, exclusive of the group wherein hydrogen bonded to the carbon atom at α-position relative to the sulfo group is substituted by fluorine or fluoroalkyl.

The C₁-C₄₀ hydrocarbyl group R⁵¹ may be saturated or unsaturated and straight, branched or cyclic. Examples thereof include C₁-C₄₀ alkyl groups such as methyl, ethyl, propyl, isopropyl, n-butyl, sec-butyl, tert-butyl, n-pentyl, tert-pentyl, n-hexyl, n-octyl, 2-ethylhexyl, n-nonyl, and n-decyl; C₂-C₄₀ alkenyl groups such as vinyl, 1-propenyl, 2-propenyl, butenyl and hexenyl; C₃-C₄₀ unsaturated alicyclic hydrocarbyl groups such as cyclohexenyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decyl, adamantyl and adamantylmethyl; C₆-C₄₀ aryl groups such as phenyl, naphthyl, alkylphenyl groups (e.g., 2-methylphenyl, 3-methylphenyl, 4-methylphenyl, 4-ethylphenyl, 4-tert-butylphenyl, 4-n-butylphenyl), di- or trialkylphenyl groups (e.g., 2,4-dimethylphenyl and 2,4,6-triisopropylphenyl), alkylnaphthyl groups (e.g., methylnaphthyl and ethylnaphthyl), and dialkylnaphthyl groups (e.g., dimethylnaphthyl and diethylnaphthyl), and C₇-C₄₀ aralkyl groups such as benzyl, 1-phenylethyl and 2-phenylethyl.

In the hydrocarbyl group, some or all hydrogen may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, thioether bond, ester bond, sulfonic ester bond, carbonate bond, lactone ring, sultone ring, carboxylic anhydride (—C(═O)—O—C(═O)—), or haloalkyl moiety. Suitable heteroatom-containing hydrocarbyl groups include heteroaryl groups such as thienyl; alkoxyphenyl groups such as 4-hydroxyphenyl, 4-methoxyphenyl, 3-methoxyphenyl, 2-methoxyphenyl, 4-ethoxyphenyl, 4-tert-butoxyphenyl and 3-tert-butoxyphenyl; alkoxynaphthyl groups such as methoxynaphthyl, ethoxynaphthyl, n-propoxynaphthyl and n-butoxynaphthyl; dialkoxynaphthyl groups such as dimethoxynaphthyl and diethoxynaphthyl; and aryloxoalkyl groups, typically 2-aryl-2-oxoethyl groups such as 2-phenyl-2-oxoethyl, 2-(1-naphthyl)-2-oxoethyl and 2-(2-naphthyl)-2-oxoethyl.

In formula (E2), R⁵² is a C₁-C₄₀ hydrocarbyl group which may contain a heteroatom. Examples of the hydrocarbyl group R⁵² are as exemplified above for the hydrocarbyl group R⁵¹. Other examples include fluorinated alkyl groups such as trifluoromethyl, trifluoroethyl, 2,2,2-trifluoro-1-methyl-1-hydroxyethyl, and 2,2,2-trifluoro-1-(trifluoromethyl)-1-hydroxyethyl; and fluorinated aryl groups such as pentafluorophenyl and 4-trifluoromethylphenyl.

Examples of the anion in the sulfonic acid salt having formula (E1) are shown below, but not limited thereto.

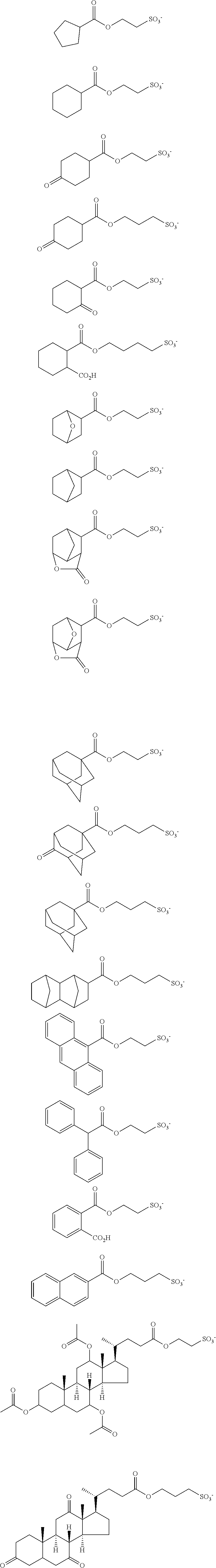

Examples of the anion in the carboxylic acid salt having formula (E2) are shown below, but not limited thereto.

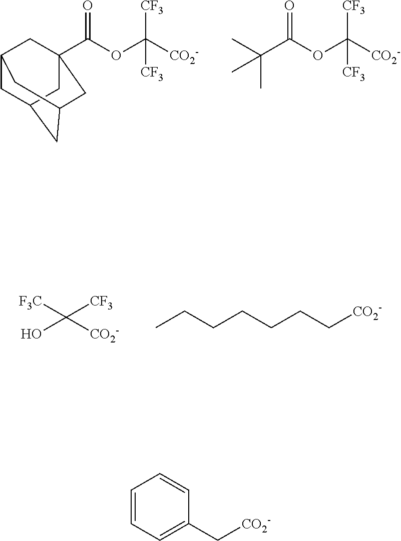

In formulae (E1) and (E2), Mq⁺ is an onium cation. The onium cation is typically selected from sulfonium, iodonium, and ammonium cations. As the sulfonium cation, those having formula (Z-1) are preferred, and examples thereof are as exemplified above for the sulfonium cation having formula (Z-1). As the iodonium cation, those having formula (Z-2) are preferred, and examples thereof are as exemplified above for the iodonium cation having formula (Z-2).

When the chemically amplified resist composition contains the quencher, the amount thereof is preferably 0.001 to 12 parts by weight, more preferably 0.01 to 8 parts by weight per 80 parts by weight of the base polymer. When the quencher is blended, the sensitivity of a resist film can be easily adjusted, the diffusion of acid in the resist film is controlled, which leads to an improvement in resolution, minimization of sensitivity changes after exposure, mitigation of substrate or environment dependency, and improvements in exposure latitude and pattern profile. The addition of the quencher is also effective for enhancing the substrate adhesion. The quencher may be used alone or in admixture.

### <Surfactant>

The resist composition may further include a surfactant. Preferred are a surfactant which is insoluble or substantially insoluble in water and soluble in alkaline developer, and a surfactant which is insoluble or substantially insoluble in water and alkaline developer. For the surfactant, reference should be made to the definition of component (S) in JP-A 2010-215608 and JP-A 2011-016746.

While many examples of the surfactant which is insoluble or substantially insoluble in water and alkaline developer are described in the patent documents cited herein, preferred examples are surfactants FC-4430 (3M), Olfine® E1004 (Nissin Chemical Co., Ltd.), Surflon® 5-381, KH-20 and KH-30 (AGC Seimi Chemical Co., Ltd.). Partially fluorinated oxetane ring-opened polymers having the formula (surf-1) are also useful.

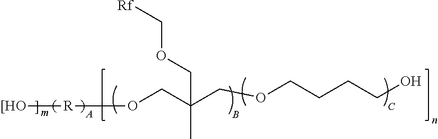

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,2-propanediyl, 1,4-butanediyl, 2,2-dimethyl-1,3-propanediyl and 1,5-pentanediyl. Exemplary tri- and tetra-valent groups are shown below.

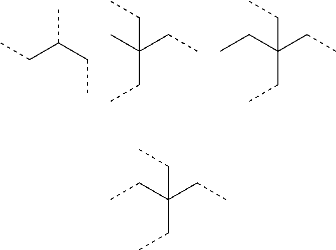

Herein the broken line denotes a valence bond. These formulae are partial structures derived from glycerol, trimethylol ethane, trimethylol propane, and pentaerythritol, respectively. Of these, 1,4-butanediyl and 2,2-dimethyl-1,3-propanediyl are preferably used.

Rf is trifluoromethyl or pentafluoroethyl, and preferably trifluoromethyl. The letter m is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and C is 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

A further embodiment of the invention is a method for preparing a chemically amplified resist composition comprising the step of dissolving starting compounds comprising a base polymer containing the polymer obtained from the aforementioned method and the PAG in the organic solvent and mixing. The starting compounds may contain at least one compound selected from the fluorinated polymer, the quencher and the surfactant, if necessary.

The starting compounds are added to the organic solvent simultaneously or in any desired order, dissolved and mixed such that the contents of respective components may fall in the aforementioned ranges.

After mixing, the solution is preferably passed through a filter. Suitable materials of which the filter is made include fluorocarbon, cellulose, nylon, polyester, and hydrocarbon base materials. Preferred for the filtration of a chemically amplified resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 200 nm. A single filter may be used or a plurality of filters may be used in combination. Although the filtering method may be single pass of the solution, preferably the filtering step is repeated by circulating the solution.

### [Pattern Forming Process]

A further embodiment of the invention is a pattern forming process comprising the steps of applying the chemically amplified resist composition onto a substrate to form a resist film thereon, exposing the resist film to high-energy radiation, and developing the exposed resist film in a developer.

As the substrate, for example, a substrate on which an integrated circuit is to be formed (e.g., Si, SiO₂, SiN, SiON, TiN, WSi, BPSG, SOG, or organic antireflective coating) or a substrate on which a mask circuit is to be formed (e.g., Cr, CrO, CrON, MoSi₂, or SiO₂) may be used.

The resist film may be formed by applying the chemically amplified resist composition onto a substrate by such means as spin coating, and prebaking the coating on a hotplate preferably at a temperature of 60 to 150° C. for 1 to 10 minutes, more preferably at 80 to 140° C. for 1 to 5 minutes. The resulting resist film is generally 0.05 to 2 μm thick.

Examples of the high-energy radiation used for the exposure of a resist film include KrF excimer laser radiation, ArF excimer laser radiation, EUV of wavelength 3 to 15 nm, and EB. When KrF excimer laser radiation, ArF excimer laser radiation or EUV is used, the resist film is exposed thereto through a mask having a desired pattern in a dose of preferably 1 to 200 mJ/cm², more preferably 10 to 100 mJ/cm². When EB is used, the resist film is exposed thereto directly or through a mask having a desired pattern in a dose of preferably 0.1 to 100 μC/cm², more preferably 0.5 to 50 μC/cm².

The exposure may be performed by conventional lithography whereas the immersion lithography of holding a liquid having a refractive index of at least 1.0 between the resist film and the projection lens may be employed. The liquid is typically water, and in this case, a protective film which is insoluble in water may be formed on the resist film.

While the water-insoluble protective film serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate preferably at 60 to 150° C. for 1 to 5 minutes, more preferably at 80 to 140° C. for 1 to 3 minutes.

The resist film is then developed with a developer in the form of an aqueous base solution, for example, 0.1 to 5 wt %, preferably 2 to 3 wt % aqueous solution of tetramethylammonium hydroxide (TMAH) for 0.1 to 3 minutes, preferably 0.5 to 2 minutes by conventional techniques such as dip, puddle and spray techniques. In this way, a desired resist pattern is formed on the substrate.

Also, after the resist film is formed, a step of rinsing with pure water may be introduced to extract the acid generator or the like from the film surface or wash away particles. After exposure, a step of rinsing may be introduced to remove any water remaining on the film after exposure.

In the pattern forming process, negative tone development may also be used. That is, an organic solvent may be used instead of the aqueous alkaline solution as the developer for developing and dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. These organic solvents may be used alone or in admixture of two or more.

### EXAMPLES

Examples and Comparative Examples are given below by way of illustration and not by way of limitation. It is noted that Mw is measured by GPC versus polystyrene standards using N,N-dimethylformamide solvent. The term “pbw” stands for parts by weight.

The reagents used in Examples are shown below.

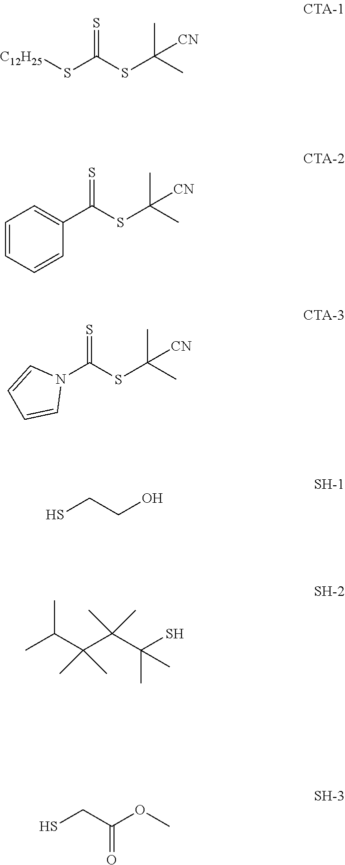

I-1: dimethyl 2,2′-azobisisobutyrate
I-2: 2,2′-azobisisobutyronitrile

Monomers MA-1 to MA-6, MB-1, MB-2, and MC-1 to MC-3 used in Examples are shown below.

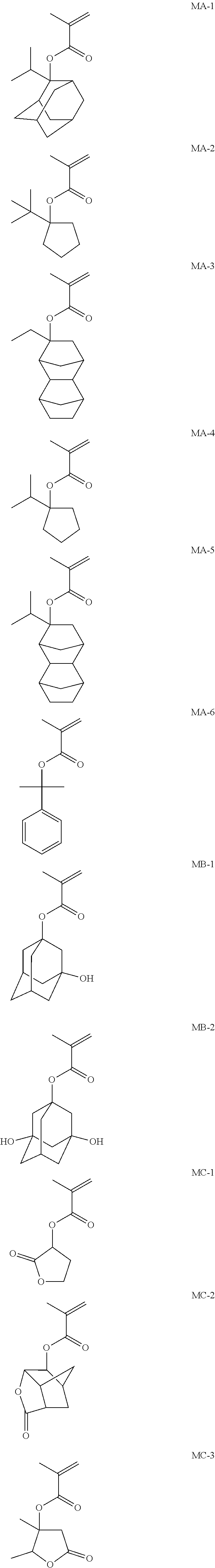

### [1] Preparation of Polymer

### Example 1-1

### Preparation of Polymer P-1 (Dropwise Addition Procedure)

Solution A was prepared under nitrogen atmosphere by dissolving 17.5 g of MA-1 (50 wt % PGMEA solution), 2.0 g of MA-2, 1.1 g of MB-1, and 8.2 g of MC-1 in 7.9 g of PGMEA, and repeating 3 cycles of vacuum pumping for 20 minutes and nitrogen purging. Separately, Solution B was prepared by dissolving 0.43 g of I-1 in 1.9 g of PGMEA, and 15 repeating 3 cycles of vacuum pumping for 20 minutes and nitrogen purging. Further, Solution C was prepared by dissolving 1.97 g of CTA-1 in 18.5 g of PGMEA, and repeating 3 cycles of vacuum pumping for 20 minutes and nitrogen purging. The reactor containing Solution C was heated until the internal temperature reached 70° C. Using syringe pumps, Solution A and Solution B were independently added dropwise to the reactor over 2 hours. At the end of addition, the polymerization solution was stirred for 4 hours while keeping the temperature of 70° C. and then cooled to room temperature. The foregoing procedure is referred to as step RM-1. The reaction solution of step RM-1 had a solid concentration of 35% by weight.

Subsequently, Solution D was prepared by dissolving 1.31 g of I-1 and 0.71 g of SH-1 in 9.6 g of PGMEA, and repeating 3 cycles of vacuum pumping for 20 minutes and nitrogen purging. Using a syringe pump, Solution D was added dropwise to the reaction solution of step RM-1 over 5 minutes. At the end of addition, the reactor was heated again until the internal temperature reached 70° C. The solution was stirred for 3 hours while keeping the temperature of 70° C. and then cooled to room temperature. The foregoing procedure is referred to as step RM-2.

Thereafter, the solution of step RM-2 was diluted with 13.4 g of PGMEA to a concentration of 25% by weight. The dilution was added dropwise to 320 g of methanol. The precipitated solids were collected by filtration and dried in vacuum at 50° C. for 20 hours, obtaining Polymer P-1 as white solids (amount 18.0 g, yield 90%).

Polymer P-1 had a Mw of 7,800 and a Mw/Mn of 1.18. The end conversion rate was 100%.

### Examples 1-2 to 1-6, Comparative Example 1-1, and Comparative Example 1-5

Polymers P-2 to P-5, CP-1 and CP-5 were prepared by the same procedure as Example 1-1 except that the type and compositional ratio of monomers were changed.

### Example 1-7

### Preparation of Polymer P-7 (Bulk Addition Procedure)

Solution A was prepared in a reactor under nitrogen atmosphere by dissolving 17.5 g of MA-1 (50 wt % PGMEA solution), 2.0 g of MA-2, 1.1 g of MB-1, 8.2 g of MC-1, and 1.97 g of CTA-1 in 37 g of PGMEA, and repeating 3 cycles of vacuum pumping for 20 minutes and nitrogen purging. 0.43 g of I-1 was admitted to Solution A. After the completion of dissolution at room temperature was confirmed, the reactor was heated until the internal temperature reached 70° C. The polymerization solution was stirred for 8 hours while keeping the temperature of 70° C. and then cooled to room temperature. The foregoing procedure is referred to as step RM-1. The reaction solution at this point of time had a solid concentration of 35% by weight.

Subsequently, Solution B was prepared by dissolving 1.49 g of I-1 and 2.71 g of SH-1 in 9.6 g of PGMEA and repeating 3 cycles of vacuum pumping for 20 minutes and nitrogen purging. Using a syringe pump, Solution B was added dropwise to the reaction solution of step RM-1 over 5 minutes. At the end of addition, the reactor was heated again until the internal temperature reached 70° C. The solution was stirred for 3 hours while keeping the temperature of 70° C. and then cooled to room temperature. The foregoing procedure is referred to as step RM-2.

Thereafter, the solution of step RM-2 was diluted with 13.4 g of PGMEA to a concentration of 25% by weight. The dilution was added dropwise to 320 g of methanol. The precipitated solids were collected by filtration and dried in vacuum at 50° C. for 20 hours, obtaining Polymer P-7 as white solids (amount 17.5 g, yield 88%).

Polymer P-7 had a Mw of 7,800 and a Mw/Mn of 1.35. The end conversion rate was 96%.

### Comparative Example 1-2

### Preparation of Polymer CP-2

Solution A was prepared in a reactor under nitrogen atmosphere by dissolving 9.2 g of MA-5, 2.4 g of MB-1, and 8.4 g of MC-3 in 16.7 g of PGMEA, and repeating 3 cycles of vacuum pumping for 20 minutes and nitrogen purging. Separately, Solution B was prepared by dissolving 0.43 g of I-1 in 1.9 g of PGMEA, and repeating 3 cycles of vacuum pumping for 20 minutes and nitrogen purging. Further, Solution C was prepared by dissolving 1.97 g of CTA-1 in 18.5 g of PGMEA, and repeating 3 cycles of vacuum pumping for 20 minutes and nitrogen purging. The reactor containing Solution C was heated until the internal temperature reached 70° C. Using syringe pumps, Solution A and Solution B were independently added dropwise to the reactor over 2 hours. At the end of addition, the polymerization solution was stirred for 4 hours while keeping the temperature of 70° C. and then cooled to room temperature. The reaction solution at this point of time had a solid concentration of 35% by weight.

Subsequently, Solution D was prepared by dissolving 1.21 g of SH-3 in 9.6 g of PGMEA, and repeating 3 cycles of vacuum pumping for 20 minutes and nitrogen purging. Using a syringe pump, Solution D was added dropwise to the reaction solution over 5 minutes. At the end of addition, the reactor was heated again until the internal temperature reached 70° C. The solution was stirred for 3 hours while keeping the temperature of 70° C. and then cooled to room temperature.

Thereafter, the solution was diluted with 13.4 g of PGMEA to a concentration of 25% by weight. The dilution was added dropwise to 320 g of methanol. The precipitated solids were collected by filtration and dried in vacuum at 50° C. for 20 hours, obtaining Polymer CP-2 as white solids (amount 17.6 g, yield 88%).

Polymer CP-2 had a Mw of 7,600 and a Mw/Mn of 1.40. The end conversion rate was 72%.

### Comparative Example 1-3

### Preparation of Polymer CP-3

Solution A was prepared in a reactor under nitrogen atmosphere by dissolving 10.0 g of MA-5, 2.0 g of MB-1, 5.1 g of MC-1, and 2.9 g of MC-2 in 16.7 g of PGMEA, and repeating 3 cycles of vacuum pumping for 20 minutes and nitrogen purging. Separately, Solution B was prepared by dissolving 0.43 g of I-1 in 1.9 g of PGMEA, and repeating 3 cycles of vacuum pumping for 20 minutes and nitrogen purging. Further, Solution C was prepared by dissolving 1.97 g of CTA-1 in 18.5 g of PGMEA, and repeating 3 cycles of vacuum pumping for 20 minutes and nitrogen purging. The reactor containing Solution C was heated until the internal temperature reached 70° C. Using syringe pumps, Solution A and Solution B were independently added dropwise to the reactor over 2 hours. At the end of addition, the polymerization solution was stirred for 4 hours while keeping the temperature of 70° C. and then cooled to room temperature. The reaction solution at this point of time had a solid concentration of 35% by weight.

Subsequently, Solution D was prepared by dissolving 1.31 g of I-1 in 9.6 g of PGMEA, and repeating 3 cycles of vacuum pumping for 20 minutes and nitrogen purging. Using a syringe pump, Solution D was added dropwise to the reaction solution over 5 minutes. At the end of addition, the reactor was heated again until the internal temperature reached 70° C. The solution was stirred for 3 hours while keeping the temperature of 70° C. and then cooled to room temperature.

Thereafter, the solution was diluted with 13.4 g of PGMEA to a concentration of 25% by weight. The dilution was added dropwise to 320 g of methanol. The precipitated solids were collected by filtration and dried in vacuum at 50° C. for 20 hours, obtaining Polymer CP-3 as white solids (amount 19.0 g, yield 95%).

Polymer CP-3 had a Mw of 7,800 and a Mw/Mn of 1.38. The end conversion rate was 84%.

### Comparative Example 1-4

### Preparation of Polymer CP-4

Solution A was prepared in a reactor under nitrogen atmosphere by dissolving 3.7 g of MA-3, 6.2 g of MA-4, 2.1 g of MB-1, and 8.0 g of MC-2 in 16.7 g of PGMEA, and repeating 3 cycles of vacuum pumping for 20 minutes and nitrogen purging. Separately, Solution B was prepared by dissolving 4.3 g of I-1 in 1.9 g of PGMEA, and repeating 3 cycles of vacuum pumping for 20 minutes and nitrogen purging. A reactor was charged with 18.5 g of PGMEA. After repeating 3 cycles of vacuum pumping for 20 minutes and nitrogen purging, the reactor was heated until the internal temperature reached 70° C. Using syringe pumps, Solution A and Solution B were independently added dropwise to the reactor over 2 hours. At the end of addition, the polymerization solution was stirred for 4 hours while keeping the temperature of 70° C. and then cooled to room temperature. The reaction solution at this point of time had a solid concentration of 35% by weight.

Thereafter, the solution of step RM-2 was diluted with 23 g of PGMEA to a concentration of 25% by weight. The dilution was added dropwise to 320 g of methanol. The precipitated solids were collected by filtration and dried in vacuum at 50° C. for 20 hours, obtaining Polymer CP-4 as white solids (amount 18.1 g, yield 91%).

Polymer CP-4 had a Mw of 7,300 and a Mw/Mn of 1.68.

Table 1 tabulates the starting materials and post-treatment conditions for the polymers prepared in Examples and Comparative Examples. Table 2 tabulates the analytical values, end conversion rate, Mw and Mw/Mn midway each step. The stage at the end of step (1) is referred to as RM-1, the stage at the end of step (2) is referred to as RM-2, and the stage at the end of step (3) is referred to after purification.

TABLE 1

| | | | | | | | | | Chain | | |
|---|---|---|---|---|---|---|---|---|---|---|---|
| | | | Monomer | Monomer | Monomer | Monomer | Monomer | | transfer | | |
| | | | A1 | A2 | B | C1 | C2 | Initiator I | agent | | |
| | | | (molar | (molar | (molar | (molar | (molar | (molar | (molar | Initiator | |
| | | Polymer | ratio) | ratio) | ratio) | ratio) | ratio) | ratio) | ratio) | Thiol | Procedure |
| Example | 1-1 | P-1 | MA-1 | MA-2 | MB-1 | MC-1 | — | I-1 | CTA-1 | I-1 | dropwise |
| | | | (35) | (10) | (5) | (50) | | (2) | (6) | SH-1 | |
| | 1-2 | P-2 | MA-3 | MA-4 | MB-1 | MC-1 | MC-2 | I-2 | CTA-2 | I-2 | dropwise |
| | | | (15) | (35) | (10) | (20) | (20) | (2) | (6) | SH-1 | |
| | 1-3 | P-3 | MA-3 | MA-4 | MB-1 | MC-1 | MC-2 | I-2 | CTA-1 | I-2 | dropwise |
| | | | (15) | (35) | (10) | (20) | (20) | (2) | (6) | SH-1 | |
| | 1-4 | P-4 | MA-3 | MA-4 | MB-1 | MC-1 | MC-2 | I-2 | CTA-1 | I-2 | dropwise |
| | | | (15) | (35) | (10) | (20) | (20) | (2) | (6) | SH-1 | |
| | 1-5 | P-5 | MA-5 | — | MB-2 | MC-2 | MC-3 | I-2 | CTA-1 | I-2 | dropwise |
| | | | (40) | | (10) | (20) | (30) | (2) | (6) | SH-2 | |
| | 1-6 | P-6 | MA-1 | MA-4 | — | MC-2 | MC-3 | I-1 | CTA-1 | I-1 | dropwise |
| | | | (15) | (35) | | (25) | (25) | (2) | (6) | SH-3 | |
| | 1-7 | P-7 | MA-1 | MA-2 | MB-1 | MC-1 | — | I-1 | CTA-1 | I-1 | bulk |
| | | | (35) | (10) | (5) | (50) | | (2) | (6) | SH-1 | |
| Comparative | 1-1 | CP-1 | MA-1 | MA-4 | — | MC-2 | MC-3 | I-1 | CTA-3 | I-1 | dropwise |
| Example | | | (15) | (35) | | (25) | (25) | (2) | (6) | SH-3 | |
| | 1-2 | CP-2 | MA-5 | — | MB-1 | MC-3 | — | I-1 | CTA-2 | — | dropwise |
| | | | (38) | | (12) | (50) | | (2) | (6) | SH-3 | |
| | 1-3 | CP-3 | MA-5 | — | MB-1 | MC-1 | MC-2 | I-1 | CTA-1 | I-1 | dropwise |
| | | | (40) | | (10) | (35) | (15) | (2) | (6) | — | |
| | 1-4 | CP-4 | MA-3 | MA-4 | MB-1 | MC-2 | — | I-1 | — | — | — |
| | | | (15) | (35) | (10) | (40) | | (20) | | — | |
| | 1-5 | CP-5 | MA-5 | MA-6 | MB-1 | MC-2 | MC-3 | I-2 | CTA-2 | I-2 | dropwise |
| | | | (20) | (10) | (10) | (30) | (30) | (2) | (6) | SH-3 | |

TABLE 2

| | | | Step RM-2 | Step RM-2 | Step RM-2 | Step RM-2 | Step RM-2 | | Total | | |
|---|---|---|---|---|---|---|---|---|---|---|---|
| | | | Amount of | Amount of | Amount of | Amount of | Amount of | | monomer | | |
| | | | residual | residual | residual | residual | residual | End | content | | |
| | | | monomer | monomer | monomer | monomer | monomer | conversion | after | Mw | Mw/Mn |
| | | | (molar | (molar | (molar | (molar | (molar | rate | purification | after | after |
| | | Polymer | ratio) | ratio) | ratio) | ratio) | ratio) | (%) | (%) | purification | purification |
| Example | 1-1 | P-1 | MA-1 | MA-2 | MB-1 | MC-1 | — | 100 | <0.01 | 7,800 | 1.18 |
| | | | (<0.01) | (<0.01) | (<0.01) | (<0.01) | | | | | |
| | 1-2 | P-2 | MA-3 | MA-4 | MB-1 | MC-1 | MC-2 | 100 | <0.01 | 7,300 | 1.14 |
| | | | (<0.01) | (<0.01) | (<0.01) | (<0.01) | (0.01) | | | | |
| | 1-3 | P-3 | MA-3 | MA-4 | MB-1 | MC-1 | MC-2 | 100 | <0.01 | 7,300 | 1.15 |
| | | | (<0.01) | (<0.01) | (<0.01) | (<0.01) | (0.01) | | | | |
| | 1-4 | P-4 | MA-3 | MA-4 | MB-1 | MC-1 | MC-2 | 100 | <0.01 | 7,300 | 1.16 |
| | | | (<0.01) | (<0.01) | (<0.01) | (<0.01) | (0.01) | | | | |
| | 1-5 | P-5 | MA-5 | — | MB-2 | MC-2 | MC-3 | 100 | <0.01 | 7,500 | 1.20 |
| | | | (<0.01) | | (<0.01) | (0.01) | (<0.01) | | | | |
| | 1-6 | P-6 | MA-1 | MA-4 | — | MC-2 | MC-3 | 100 | <0.01 | 7,000 | 1.25 |
| | | | (<0.01) | (<0.01) | | (<0.01) | (<0.01) | | | | |
| | 1-7 | P-7 | MA-1 | MA-2 | MB-1 | MC-1 | — | 100 | <0.01 | 7,800 | 1.35 |
| | | | (<0.01) | (<0.01) | (<0.01) | (<0.01) | | | | | |
| Com- | 1-1 | CP-1 | MA-1 | MA-4 | — | MC-2 | MC-3 | 85 | <0.01 | 7,700 | 1.34 |
| parative | | | (<0.01) | (<0.01) | | (0.02) | (0.01) | | | | |
| Example | 1-2 | CP-2 | MA-5 | — | MB-1 | MC-3 | — | 72 | 0.31 | 7,600 | 1.40 |
| | | | (0.13) | | (0.15) | (0.15) | | | | | |
| | 1-3 | CP-3 | MA-5 | — | MB-1 | MC-1 | MC-2 | 84 | 0.02 | 7,800 | 1.38 |
| | | | (<0.01) | | (0.01) | (0.02) | (0.03) | | | | |
| | 1-4 | CP-4 | — | — | — | — | — | — | 0.51 | 7,300 | 1.68 |
| | 1-5 | CP-5 | MA-5 | MA-6 | MB-1 | MC-2 | MC-3 | 100 | <0.01 | 8,000 | 1.20 |
| | | | (<0.01) | (0.03) | (<0.01) | (<0.01) | (<0.01) | | | | |

As is evident from the results shown in Tables 1 and 2, polymers having a narrow dispersity in which the removal of end groups originating from RAFT agents has been almost completed are obtained according to the inventive method. In particular, polymers obtained from dropwise polymerization have a better Mw/Mn than that of bulk polymerization, that is, a narrow dispersity. It is demonstrated that the polymer preparation method of the invention is effective for completing the removal of end groups which is insufficiently achieved by the prior art living radical polymerization methods.

### [2] Preparation of Polymer Solution and Evaluation of Age Stability

### Example 2-1

Polymer P-1 in wet powder form was prepared by performing the preparation procedure according to the method of Example 1-1, adding dropwise the polymerization solution to diisopropyl ether, and filtering the precipitated solids. The wet powder was placed in a flask, PGME was added to the flask to dissolve the powder, and the solution was concentrated in vacuum at 40° C. to remove diisopropyl ether. After concentration, the amount of PGME was quantitatively determined by gas chromatography. A suitable amount of PGMEA was added, obtaining a PGME/PGMEA solution of Polymer P-1 (polymer concentration 10 wt %, PGME:PGMEA=70:30 in weight ratio). The solution was passed through a conduit having a nylon filter with a pore size of 5 nm and a polyethylene filter with a pore size of 1 nm arranged therein in the described order, obtaining the polymer solution.

### Example 2-2

Polymer P-3 in wet powder form was prepared by performing the preparation procedure according to the method of Example 1-3, adding dropwise the polymerization solution to diisopropyl ether, and filtering the precipitated solids. The wet powder was placed in a flask, PGME was added to the flask to dissolve the powder, and the solution was concentrated in vacuum at 40° C. to remove diisopropyl ether. After concentration, the amount of PGME was quantitatively determined by gas chromatography. A suitable amount of PGMEA was added, obtaining a PGME/PGMEA solution of Polymer P-3 (polymer concentration 10 wt %, PGME:PGMEA=70:30 in weight ratio). The solution was passed through a conduit having a nylon filter with a pore size of 5 nm and a polyethylene filter with a pore size of 1 nm arranged therein in the described order, obtaining the polymer solution.

### Comparative Example 2-1

Polymer CP-1 in wet powder form was prepared by performing the preparation procedure according to the method of Comparative Example 1-1, adding dropwise the polymerization solution to diisopropyl ether, and filtering the precipitated solids. The wet powder was placed in a flask, PGME was added to the flask to dissolve the powder, and the solution was concentrated in vacuum at 40° C. to remove diisopropyl ether. After concentration, the amount of PGME was quantitatively determined by gas chromatography. A suitable amount of PGMEA was added, obtaining a PGME/PGMEA solution of Polymer CP-1 (polymer concentration 10 wt %, PGME:PGMEA=70:30 in weight ratio). The solution was passed through a conduit having a nylon filter with a pore size of 5 nm and a polyethylene filter with a pore size of 1 nm arranged in the described order, obtaining the polymer solution.

### Comparative Example 2-2

Polymer CP-3 in wet powder form was prepared by performing the preparation procedure according to the method of Comparative Example 1-3, adding dropwise the polymerization solution to diisopropyl ether, and filtering the precipitated solids. The wet powder was placed in a flask, PGME was added to the flask to dissolve the powder, and the solution was concentrated in vacuum at 40° C. to remove diisopropyl ether. After concentration, the amount of PGME was quantitatively determined by gas chromatography. A suitable amount of PGMEA was added, obtaining a PGME/PGMEA solution of Polymer CP-3 (polymer concentration 10 wt %, PGME:PGMEA=70:30 in weight ratio). The solution was passed through a conduit having a nylon filter with a pore size of 5 nm and a polyethylene filter with a pore size of 1 nm arranged in the described order, obtaining the polymer solution.

Two brown glass bottles were filled with each of the polymer solutions of Examples 2-1 and 2-2 and Comparative Examples 2-1 and 2-2. The bottles were stored for 2 weeks, with one in an environment at 5° C. and the other at 40° C. After storage at different temperatures, the polymer solutions were allowed to stand in an environment at 23° C. for 8 hours. Using coater/developer Clean Track® Lithius Pro Z (Tokyo Electron Ltd.), the polymer solution was coated onto a 12-inch silicon wafer substrate and baked on a hotplate at 130° C. for 60 seconds to form a polymer film. The film was measured for thickness using spectroscopic film thickness measurement system VM-3500 (Screen Semiconductor Solutions Co., Ltd.). On Clean Track® Lithius Pro Z, the polymer film after the thickness measurement was developed for 30 seconds in total by spinning the wafer and dispensing a 2.38 wt % solution of tetramethylammonium hydroxide. The alkaline solution was washed away with water, and the wafer was rotated at a high speed to spin off water. The thickness of the polymer film after development was measured. The difference of film thickness before and after development treatment was determined. The measured film thickness is shown in Table 3.

TABLE 3

| | | | | Film thickness | Film thickness | Film thickness |
|---|---|---|---|---|---|---|
| | | | Storage | after coating | after development | difference |
| | | Polymer | temp. | (Å) | (Å) | (Å) |
| Example | 2-1 | P-1 | 5° C. | 3,280 | 3,265 | 15 |
| | | | 40° C. | 3,281 | 3,265 | 16 |
| | 2-2 | P-3 | 5° C. | 3,150 | 3,133 | 17 |
| | | | 40° C. | 3,150 | 3,134 | 16 |
| Comparative | 2-1 | CP-1 | 5º C. | 3,300 | 3,284 | 16 |
| Example | | | 40° C. | 3,301 | 3,260 | 41 |
| | 2-2 | CP-3 | 5° C. | 3,151 | 3,141 | 10 |
| | | | 40° C. | 3,150 | 3,084 | 66 |

The polymers of Examples having a high end conversion rate kept the film thickness difference before and after development treatment unchanged even when the storage temperature was 40° C. The polymers of Comparative Examples having a low end conversion rate experienced a substantial film thickness difference when the storage temperature was 40° C. In the polymer solution during 40° C. storage, the dissociation of functional groups (originating from the RAFT agent) from the polymer end takes place along with decomposition thereof. Then an organic acid component is created in the polymer solution, which cause decomposition of acid labile groups on the polymer to promote the dissolution of the polymer in the alkaline developer. It is seen from the results of Table 3 that the polymers with a high end conversion rate are superior in stability with time.

### [3] Preparation and evaluation of chemically amplified resist composition

### Examples 3-1 to 3-7 and Comparative Examples 3-1 to 3-5

### (1) Preparation of Chemically Amplified Resist Composition

A chemically amplified resist composition was prepared by dissolving components selected from Polymers P-1 to P-7, Comparative Polymers CP-1 to CP-5, photoacid generator PAG-1, quencher Q-1, and fluorinated polymer SF-1 in an organic solvent containing 0.01 wt % of surfactant A according to the formulation shown in Table 4, and passing the solution through a Teflon® filter with a pore size of 0.2 μm.

The solvent, photoacid generator PAG-1, quencher Q-1, and fluorinated polymer SF-1 are identified below.

### Solvent:

PGMEA: propylene glycol monomethyl ether acetate
GBL: γ-butyrolactone

### Photoacid Generator PAG-1

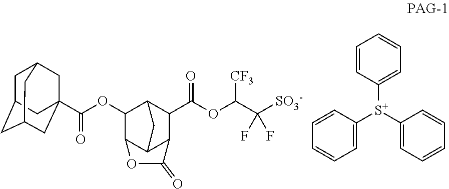

### Quencher Q-1

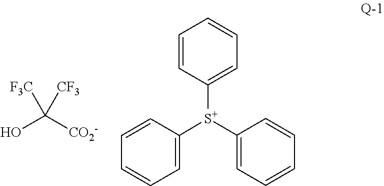

### Fluorinated Polymer SF-1

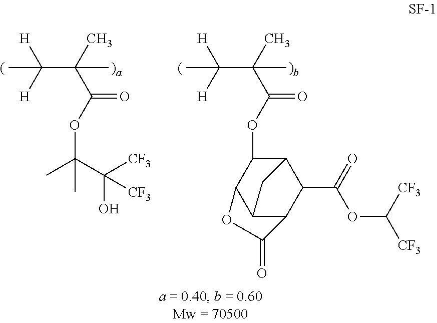 [SMILES: CCC(C)(C)C(=O)OC(C)(C)C(O)(C(F)(F)F)C(F)(F)F ; CCC(C)(C)C(=O)OC1C2CC3C1OC(=O)C3C2C(=O)OC(C(F)(F)F)C(F)(F)F]

### Surfactant A:

3-methyl-3-(2,2,2-trifluoroethoxymethyl)oxetane/tetrahydrofuran/2,2-dimethyl-1,3-propane diol copolymer (Omnova Solutions, Inc.)

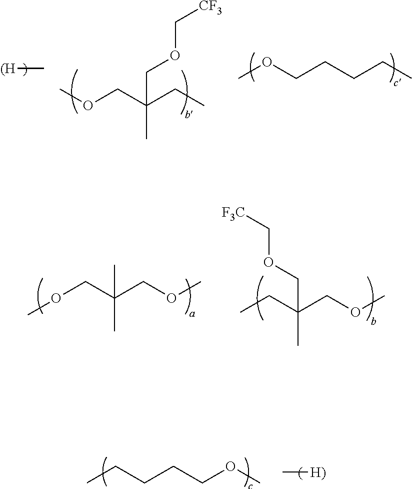

a:(b+b′):(c+c′)=1: 4-7: 0.01-1 (molar ratio)
Mw=1,500
   (2) ArF immersion lithography test

A spin-on carbon film ODL-180 (Shin-Etsu Chemical Co., Ltd.) having a carbon content of 80 wt % was deposited on a silicon substrate to a thickness of 180 nm, and a silicon-containing spin-on hard mask SHB-A941 having a silicon content of 43 wt % was deposited thereon to a thickness of 35 nm. On this substrate for trilayer process, the resist composition was spin coated and baked on a hotplate at 100° C. for 60 seconds to form a resist film of 100 nm thick.

Using an ArF excimer laser immersion lithography scanner (XT-1900Gi by ASML, NA 1.35, 3/4 annular, X-Y polarized illumination), exposure was performed through a mask bearing a contact hole (CH) pattern with a hole size of 52 nm and a pitch of 104 nm (on-wafer size) while varying the dose and focus (dose pitch: 1 mJ/cm², focus pitch: 0.025 μm). The immersion liquid used herein was water. After the exposure, the wafer was baked (PEB) at the temperature shown in Table 4 for 60 seconds. Thereafter, the resist film was puddle developed in n-butyl acetate for 30 seconds, rinsed with 4-methyl-2-pentanol, and spin dried, obtaining a CH pattern or negative pattern. The CH pattern after development was observed under CD-SEM CG-5000 (Hitachi High Technologies Corp.) whereupon sensitivity, DOF, and MEF were evaluated by the following methods. The results are shown in Table 4.

### [Evaluation of Sensitivity]

The optimum dose Eop (mJ/cm²) which provided a CH pattern with a hole diameter of 52 nm and a pitch of 104 nm was determined as an index of sensitivity.

### [Evaluation of DOF]

For the resist pattern printed at Eop, a range offocus (nm) which provided a hole pattern with a width of 52 nm+500 was determined as an index of DOF.

### [Evaluation of MEF]

A pattern was formed by exposure at the optimum dose Eop by ArF lithography patterning test with the pitch fixed and the mask size varied. MVEF was calculated from the mask size and a tilt of the hole pattern width. A value of up to 4.5 indicates acceptable MEF.

TABLE 4

| | | | Photoacid | | Fluorinated | | | | | |
|---|---|---|---|---|---|---|---|---|---|---|
| | | Polymer | generator | Quencher | polymer | Organic solvent | PEB | Sensitivity | DOF | |
| | | (pbw) | (pbw) | (pbw) | (pbw) | (pbw) | (° C.) | (mJ/cm²) | (nm) | MEF |
| Example | 3-1 | P-1 | PAG-1 | Q-1 | SF-1 | PGMEA (1550) | 95 | 23 | 120 | 4.0 |
| | | (80) | (8.0) | (4.5) | (5.0) | GBL (250) | | | | |
| | 3-2 | P-2 | PAG-1 | Q-1 | SF-1 | PGMEA (1550) | 95 | 30 | 130 | 3.5 |
| | | (80) | (8.0) | (4.5) | (5.0) | GBL (250) | | | | |
| | 3-3 | P-3 | PAG-1 | Q-1 | SF-1 | PGMEA (1550) | 95 | 30 | 130 | 3.8 |
| | | (80) | (8.0) | (4.5) | (5.0) | GBL (250) | | | | |
| | 3-4 | P-4 | PAG-1 | Q-1 | SF-1 | PGMEA (1550) | 95 | 30 | 130 | 3.8 |
| | | (80) | (8.0) | (4.5) | (5.0) | GBL (250) | | | | |
| | 3-5 | P-5 | PAG-1 | Q-1 | SF-1 | PGMEA (1550) | 95 | 30 | 130 | 3.1 |
| | | (80) | (8.0) | (4.5) | (5.0) | GBL (250) | | | | |
| | 3-6 | P-6 | PAG-1 | Q-1 | SF-1 | PGMEA (1550) | 95 | 25 | 120 | 3.2 |
| | | (80) | (8.0) | (4.5) | (5.0) | GBL (250) | | | | |
| | 3-7 | P-7 | PAG-1 | Q-1 | SF-1 | PGMEA (1550) | 95 | 31 | 105 | 4.5 |
| | | (80) | (8.0) | (4.5) | (5.0) | GBL (250) | | | | |
| Comparative | 3-1 | CP-1 | PAG-1 | Q-1 | SF-1 | PGMEA (1550) | 95 | 42 | 90 | 4.6 |
| | | (80) | (8.0) | (4.5) | (5.0) | GBL (250) | | | | |
| Example | 3-2 | CP-2 | PAG-1 | Q-1 | SF-1 | PGMEA (1550) | 95 | 30 | 90 | 5.3 |
| | | (80) | (8.0) | (4.5) | (5.0) | GBL (250) | | | | |
| | 3-3 | CP-3 | PAG-1 | Q-1 | SF-1 | PGMEA (1550) | 95 | 30 | 90 | 4.9 |
| | | (80) | (8.0) | (4.5) | (5.0) | GBL (250) | | | | |
| | 3-4 | CP-4 | PAG-1 | Q-1 | SF-1 | PGMEA (1550) | 95 | 30 | 90 | 5.5 |
| | | (80) | (8.0) | (4.5) | (5.0) | GBL (250) | | | | |
| | 3-5 | CP-5 | PAG-1 | Q-1 | SF-1 | PGMEA (1550) | 95 | 45 | 90 | 4.6 |
| | | (80) | (8.0) | (4.5) | (5.0) | GBL (250) | | | | |

Chemically amplified resist compositions comprising narrow dispersity polymers obtained from the inventive RAFT polymerization method exhibit satisfactory DOF margin and MEF. Since homogeneous polymers obtained from the inventive RAFT polymerization method and having a reduced proportion of low-molecular-weight fractions are used as the base polymer, the chemically amplified resist compositions show low acid diffusion and low exposure dependency. In contrast, Comparative Examples 3-1 and 3-3 using different chain transfer agents and monomers exhibit a low sensitivity.

As shown above, chemically amplified resist compositions comprising RAFT polymers having a high end conversion rate obtained by the polymerization and end treatment method according to the invention exhibit a high sensitivity, improved DOF and MEF and are thus suitable for use in the ArF immersion lithography. In contrast, chemically amplified resist compositions comprising conventional RAFT polymers having a low end conversion rate or polymers obtained from the free radical polymerization are unsatisfactory in either of MEF and DOF. The inventive chemically amplified resist compositions are useful in the ArF immersion lithography.

### [4] Industrial preparation of polymer and defect evaluation

### Example 5-1

### Preparation of Polymer 5-1 (Dropwise Addition Procedure)

Solution A was prepared under nitrogen atmosphere by dissolving 737.6 g of MA-2, 598.1 g of MC-1, and 338.7 g of MC-2 in 924.8 g of PGMEA, and repeating 3 cycles of vacuum pumping for 20 minutes and nitrogen purging. Separately, Solution B was prepared by dissolving 19.37 g of I-1 in 148.6 g of PGMEA, and repeating 3 cycles of vacuum pumping for 20 minutes and nitrogen purging. Further, Solution C was prepared by dissolving 72.8 g of CTA-1 in 1485.7 g of PGMEA, and repeating 3 cycles of vacuum pumping for 20 minutes and nitrogen purging. The reactor containing Solution C was heated until the internal temperature reached 70° C. Using syringe pumps, Solution A and Solution B were independently added dropwise to the reactor over 2 hours. At the end of addition, the polymerization solution was stirred for 4 hours while keeping the temperature of 70° C. and then cooled to room temperature. The foregoing procedure is referred to as step RM-1. The reaction solution of step RM-1 had a solid concentration of 35% by weight.

Subsequently, Solution D was prepared by dissolving 48.4 g of I-1 and 32.9 g of SH-1 in 761.9 g of PGMEA, and repeating 3 cycles of vacuum pumping for 20 minutes and nitrogen purging. Using a syringe pump, Solution D was added dropwise to the reaction solution of step RM-1 over 20 minutes. At the end of addition, the reactor was heated again until the internal temperature reached 70° C. The solution was stirred for 3 hours while keeping the temperature of 70° C. and then cooled to room temperature. The foregoing procedure is referred to as step RM-2.

Thereafter, the solution of step RM-2 was diluted with 1067.0 g of PGMEA to a concentration of 25% by weight. The dilution was added dropwise to 25,600 g of methanol. The precipitated solids were collected by filtration and dried in vacuum at 50° C. for 20 hours, obtaining Polymer P-1 as white solids (amount 1,312 g, yield 82%).

Polymer P-1 had a Mw of 7,400 and a Mw/Mn of 1.16. The end conversion rate was 100%. The contents of residual monomers were below the quantitative lower limit (or less than 0.01% by weight).

Polymer 5-1 was obtained in wet powder form by conducting the preparation process according to Comparative Example 5-1, adding the reaction solution dropwise to diisopropyl ether, and collecting the precipitated solids by filtration. The wet powder was admitted into a flask and dissolved in PGME. The solution was concentrated under reduced pressure at 40° C. to remove diisopropyl ether. After concentration, the content of PGME was quantitatively determined by gas chromatography. A suitable amount of PGMEA was added to the polymer to form a PGME/PGMEA solution of polymer CP-3 (polymer concentration 10 wt %, PGME:PGMEA=70:30 in weight ratio). The solution was passed through a conduit having a nylon filter with a pore size of 5 nm and a polyethylene filter with a pore size of 1 nm arranged therein in the described order, obtaining the polymer solution.

### [Measurement of Concentration of Particles in Polymer Solution]

The concentration (count/mL) of particles with a particle size of 0.15 μm or more in the polymer solution was compared. The analyzer was particle counter KS-41A (Rion Co., Ltd.). A smaller count indicates that the polymer solution contains a smaller number of particles and has a higher cleanness. The results are shown in Table 5.

TABLE 5

| | | | | | | | Concentration |
|---|---|---|---|---|---|---|---|
| | | Polymer | | | | Polymer | of ≥ 0.15 μm |
| | | preparation | | Solvent A | Solvent B | concentration | particles |
| | | method | Polymer | (wt %) | (wt %) | (wt %) | (count/mL) |
| Example | 5-1 | Example | P-1 | PGME | PGMEA | 10 | 5 |
| | | 1-1 | | (70) | (30) | | |
| | 5-2 | Example | P-3 | EL | PGMEA | 10 | 7 |
| | | 1-3 | | (60) | (40) | | |
| | 5-3 | Example | 5-1 | DAA | PGMEA | 12 | 8 |
| | | 5-1 | | (45) | (55) | | |
| Comparative | 5-1 | Comparative | CP-1 | PGME | PGMEA | 10 | 27 |
| | | Example | | (70) | (30) | | |
| | | 1-1 | | | | | |
| Example | 5-2 | Comparative | CP-3 | EL | PGMEA | 10 | 30 |
| | | Example | | (60) | (40) | | |
| | | 1-2 | | | | | |

It is evident from the results in Table 5 that polymers having a narrow dispersity and an extremely low content of residual monomers in which the removal of end groups originating from RAFT agents is complete are obtained in the industrial scale according to the polymer preparing method of the invention. It is proven that the polymer preparing method of the invention is a RAFT polymer preparation method which is applicable in an industrial scale, although prior art living radical polymerization methods are inapplicable. It is evident from the results in Table 5 that the polymer solutions obtained from the inventive method have a low concentration or count of particles, indicating a homogeneous clean solution. This is because residual monomers are reduced, which means that components having a low solubility in organic solvents are reduced. The polymers obtained from the inventive method are expected useful as a base polymer in chemically amplified resist compositions or as a resist material with a less risk of defectiveness.

Japanese Patent Application No. 2025-033041 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A method for preparing a polymer comprising repeat units adapted to be decomposed under the action of acid and free of repeat units adapted to function as a photoacid generator, the method comprising the steps of
(1) polymerizing starting monomers including a monomer having the formula (A1) in a solution through living radical polymerization using a radical initiator and a reversible addition-fragmentation chain transfer agent (RAFT agent), to form a polymer P-1 having an end structure selected from structures having the formulae (X-1) and (X-2) originating from the RAFT agent at the end of the backbone,
(2) adding a radical generator and a thiol compound to the solution containing polymer P-1, and heating the solution to convert the end structure to hydrogen to form a polymer P, and
(3) mixing the solution containing polymer P with a poor solvent and allowing polymer P to precipitate as solids for purification, 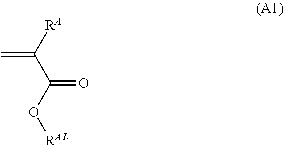
wherein R^{A} is hydrogen or methyl,
R^{AL} is an acid labile structure-containing group having any one of the following formulae (a-1) to (a-10): 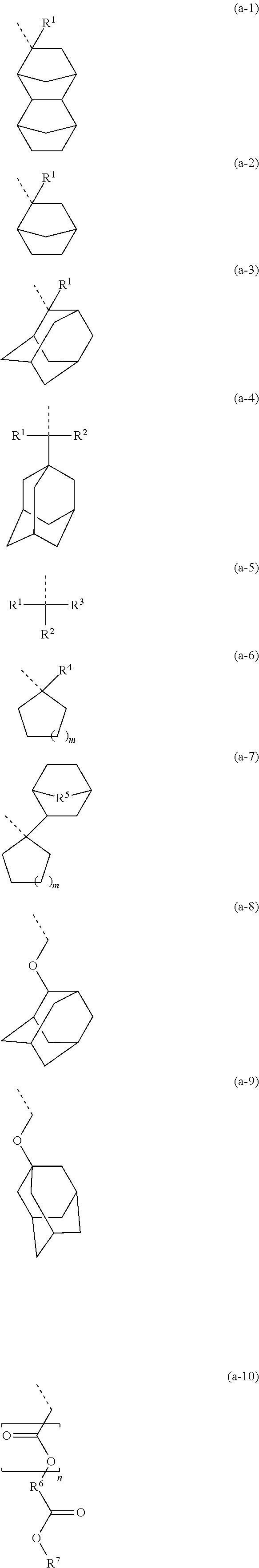
wherein R¹ and R² are each independently a C₁-C₄ alkyl group,
R³ is a C₁-C₄ alkyl group, cyclopentyl, cyclohexyl, oxanorbornyl or thianorbornyl group, some —CH₂— in the cyclopentyl and cyclohexyl groups may be replaced by —O— or —S—,
R⁴ is a C₁-C₄ alkyl group, cyclopentyl group, or cyclohexyl group,
R⁵ is —O—, —S—, methanediyl or ethane-1,2-diyl,
R⁶ is a C₃-C₄₀ divalent alicyclic group which may contain at least one atom selected from oxygen and sulfur,
R⁷ is an acid labile group having the formula (a-3), (a-5) or (a-6),
m is 1 or 2, n is 0 or 1, the broken line designates a point of attachment, 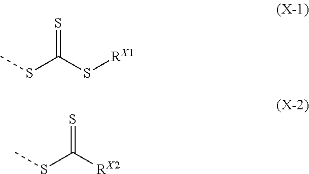
wherein R^{X1} and R^{X2} are each independently a C₂-C₂₀ saturated hydrocarbyl group which may contain a heteroatom, C₇-C₂₀ aralkyl group or C₆-C₂₀ aryl group, the broken line designates a point of attachment to the carbon atom in the backbone.

2. The method of claim 1 wherein the starting monomers further include at least one monomer selected from a monomer having the formula (A2), a monomer having the formula (A3), and a monomer having the formula (A4): 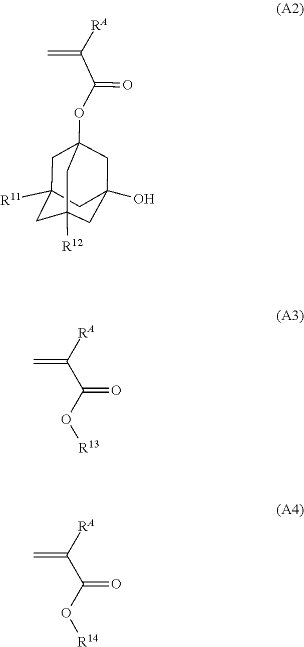
wherein R^{A} is hydrogen or methyl,
R¹ and R¹² are each independently hydrogen or hydroxy,
R¹³ is a lactone structure-containing substituent or sultone structure-containing monovalent substituent, and
R¹⁴ is hydrogen, a C₁-C₁₅ fluorinated hydrocarbyl group or C₁-C₁₅ monovalent fluoroalcohol-containing substituent.

3. The method of claim 1 wherein the starting monomers do not include a conjugated monomer.

4. The method of claim 1 wherein the thiol compound is a compound having the formula (SH-1) or (SH-2): 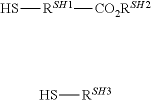
wherein R^{SH1} is a C₁-C₃ hydrocarbylene group,
R^{SH2} is a C₁-C₈ aliphatic hydrocarbyl group which may contain a heteroatom, C₇-C₁₈ aralkyl group or C₆-C₁₈ aryl group, and
R^{SH3} is a C₂-C₂₀ saturated hydrocarbyl group which may contain a heteroatom, C₇-C₁₈ aralkyl group or C₆-C₁₈ aryl group.

5. The method of claim 1 wherein in step (2), the rate of conversion of the end structure originating from the RAFT agent to hydrogen is at least 98%.

6. The method of claim 1 wherein in step (1), the amount of the radical initiator charged is 0.5 to 5 moles and the amount of the RAFT agent charged is 0.5 to 20 moles per 100 moles of all the monomers charged.

7. The method of claim 1 wherein the amounts of monomers remaining at the end of step (2) are less than 0.05% by weight based on the total weight of the monomers charged.

8. A method for preparing a chemically amplified resist composition, comprising the steps of dissolving a raw material comprising a base polymer containing the polymer obtained from the method of claim 1 and a photoacid generator in an organic solvent and mixing them.

9. The method of claim 8 wherein the photoacid generator contains at least one compound selected from a compound having the formula (B1), a compound having the formula (B2), and a compound having the formula (B3): 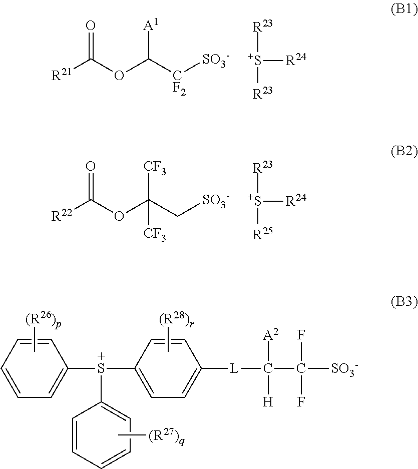
wherein A¹ is hydrogen or trifluoromethyl,
R²¹ and R²² are each independently a C₁-C₃₅ hydrocarbyl group which may contain oxygen, a C₂-C₃₅ monovalent nitrogen-containing heterocyclic group, or a group having the formula (b): 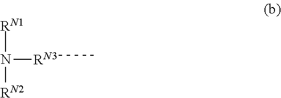
wherein R^{N1} and R^{N2} are each independently hydrogen or a C₁-C₂₀ hydrocarbyl group which may contain a heteroatom, R^{N1} and R^{N2} may bond together to form a ring with the nitrogen atom to which they are attached, R^{N3} is a C₁-C₂₀ hydrocarbylene group which may contain a heteroatom, the broken line designates a point of attachment,
R²³, R²⁴ and R²⁵ are each independently halogen or a C₁-C₃₀ hydrocarbyl group which may contain a heteroatom, any two of R²³, R²⁴ and R²⁵ may bond together to form a ring with the sulfur atom to which they are attached,
R²⁶, R²⁷ and R²⁸ are each independently halogen, a C₁-C₃₀ hydrocarbyl group which may contain a heteroatom, or a C₁-C₂₀ hydrocarbyloxy group which may contain a heteroatom,
p and q are each independently 0, 1, 2, 3, 4 or 5, r is 0, 1, 2, 3 or 4, and
L is a single bond, ether bond, or a C₁-C₂₀ hydrocarbylene group which may contain a heteroatom.

10. The method of claim 8 wherein the raw material further contains a fluorinated polymer comprising repeat units of at least one type selected from repeat units having the formula (D1), repeat units having the formula (D2), and repeat units having the formula (D3): 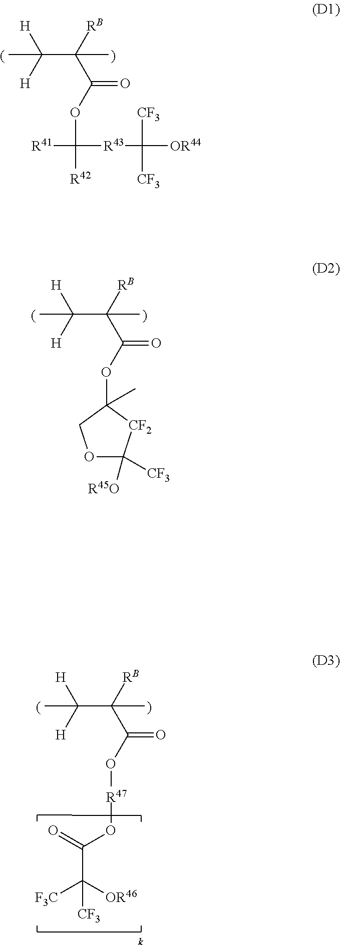
wherein R^{B} is hydrogen or methyl,
R⁴¹ and R⁴² are each independently hydrogen or a C₁-C₁₀ hydrocarbyl group,
R⁴³ is a single bond or a C₁-C₅ straight or branched hydrocarbylene group,
R⁴⁴, R⁴⁵ and R⁴⁶ are each independently hydrogen, a C₁-C₁₅ hydrocarbyl group, C₁-C₁₅ fluorinated hydrocarbyl group, C₂-C₁₅ acyl group, or acid labile group; when R⁴⁴, R⁴⁵ and R⁴⁶ are hydrocarbyl or fluorinated hydrocarbyl groups, some —CH₂— in these groups may be replaced by an ether bond or carbonyl moiety,
R⁴⁷ is a C₁-C₂₀ (k+1)-valent hydrocarbon group or C₁-C₂₀ (k+1)-valent fluorinated hydrocarbon group, and
k is 1,2 or 3.

11. The method of claim 8 wherein the raw material further contains a quencher.

12. The method of claim 8 wherein the raw material further contains a surfactant.

13. A pattern forming process comprising the steps of applying the chemically amplified resist composition obtained from the method of claim 8 onto a substrate to form a resist film thereon, exposing the resist film to high-energy radiation, and developing the exposed resist film in a developer.

14. The pattern forming process of claim 13 wherein the high-energy radiation is ArF excimer laser.

15. A polymer obtained from living radical polymerization using a reversible addition-fragmentation chain transfer agent (RAFT agent), wherein
said polymer comprises repeat units adapted to be decomposed under the action of acid and derived from a monomer having the formula (A1),
at least 98% of the end structure in the polymer, selected from structures having the formulae (X-1) and (X-2) originating from the RAFT agent has been converted to hydrogen,
the amount of the monomer having formula (A1) remaining in the polymer is up to 0.05% by weight,
the polymer has a dispersity of up to 1.50, 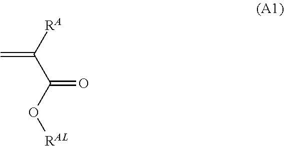
wherein R^{A} is hydrogen or methyl, and R^{AL} is an acid labile structure-containing group having any one of the formulae (a-1) to (a-10): 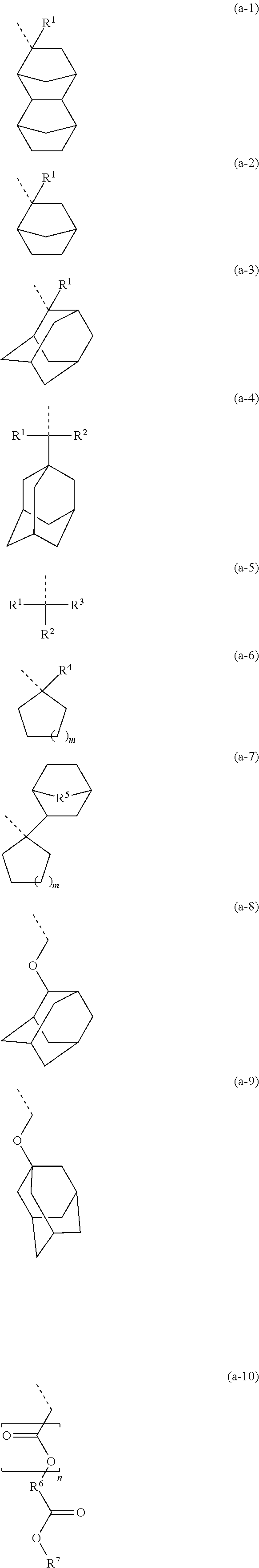
wherein R¹ and R² are each independently a C₁-C₄ alkyl group,
R³ is a C₁-C₄ alkyl group, cyclopentyl, cyclohexyl, oxanorbornyl or thianorbornyl group, some —CH₂— in the cyclopentyl and cyclohexyl groups may be replaced by —O— or —S—,
R⁴ is a C₁-C₄ alkyl group, cyclopentyl group, or cyclohexyl group,
R⁵ is —O—, —S—, methanediyl or ethane-1,2-diyl,
R⁶ is a C₃-C₄₀ divalent alicyclic group which may contain at least one atom selected from oxygen and sulfur,
R⁷ is an acid labile group having the formula (a-3), (a-5) or (a-6),
m is 1 or 2, n is 0 or 1,
the broken line designates a point of attachment, 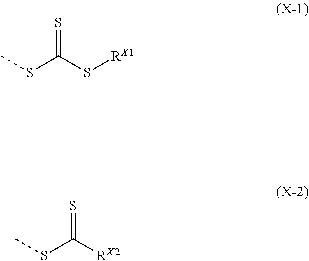
wherein R^{X1} and R^{X2} are each independently a C₂-C₂₀ saturated hydrocarbyl group which may contain a heteroatom, C₇-C₂₀ aralkyl group, or C₆-C₂₀ aryl group, the broken line designates a point of attachment to the carbon atom in the backbone.

16. The polymer of claim 15, further comprising repeat units derived from at least one monomer selected from a monomer having the formula (A2), a monomer having the formula (A3), and a monomer having the formula (A4), the amount of each monomer remaining in the polymer is up to 0.05% by weight, 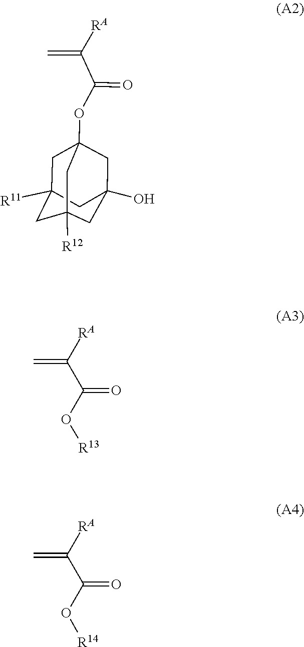
wherein R^{A} is hydrogen or methyl,
R¹¹ and R¹² are each independently hydrogen or hydroxy,
R¹³ is a lactone structure-containing substituent or sultone structure-containing monovalent substituent, and
R¹⁴ is hydrogen, a C₁-C₁₅ fluorinated hydrocarbyl group or C₁-C₁₅ monovalent fluoroalcohol-containing substituent.

17. A chemically amplified resist composition comprising a base polymer containing the polymer of claim 15 and a photoacid generator.

18. The chemically amplified resist composition of claim 17 for use in ArF lithography or ArF immersion lithography.
